(19) United States
(12) Patent Application Publication
(10) Pub. No.: US 2021/0369846 A1
(43) Pub. Date: Dec. 2, 2021

(54) New Ammonium Salts Of Fluorinated Organic Acids, Method Of Their Synthesis and Application

(71) Applicant: Nanosanguis, Warszawa (PL)

(72) Inventors: Agata Stefanek, Warszawa (PL); Tomasz Ciach, Warszawa (PL); Katarzyna Leczyckawilk, Warszawa (PL); Sylwia Czarnocka-sniadala, Warszawa (PL); Joanna Grafstein, Warszawa (PL); Magdalena Dresler, Toruh (PL); Aleksandra Nowak, Tarnow (PL); Jakub Wglinkski, Belchatow (PL)

(21) Appl. No.: 17/288,307

(22) PCT Filed: Oct. 26, 2019
(86) PCT No.: PCT/IB2019/059195

§ 371 Date: Apr. 23, 2021

(30) Foreign Application Priority Data
Oct. 26, 2018 (PL) PL427553

(51) Int. Cl. A61K 47/18; A61K 47/20; A61K 47/12; A61K 9/00

(52) U.S. Cl. CPC A61K 47/186; A61K 47/20; A61K 9/107; A61K 9/0026; A61K 47/12

(57) ABSTRACT

The exemplary arrangements relate to ammonium salts of partially fluorinated organic acids. The exemplary arrangements also relate to methods of synthesis of the ammonium salts of partially fluorinated organic acids. The exemplary arrangements also relate to methods of use of the ammonium salts of partially fluorinated organic acids to produce stabilizing emulsions of the oil-in-water and/or water-in-oil type. The exemplary arrangements also relate to methods of use and applications of the ammonium salts of partially fluorinated organic acids, for example use as stabilizing agents in blood substitute preparations.

### TECHNICAL FIELD

Exemplary arrangements relate to new ammonium salts of partially fluorinated organic acids, a method of their synthesis and methods of use of the exemplary new ammonium salts of partially fluorinated organic acids, in particular in applications in biology, biochemistry and medicine in order to possibly prolong the storage period of tissues or organs of higher organisms, as stabilising agents in blood substitute preparations.

### BACKGROUND

Partially fluorinated carboxylic acids are derived from succinic acid (butanedioic acid). Fluorinated monoester derivatives of succinic acid are obtained in a reaction of succinic anhydride with the appropriate alcohols in the presence of a mild base, e.g. Tertiary bases, such as: triethylamine (Et₃N) or Hünigs base (N,N-diisopropylethylamine), etc.

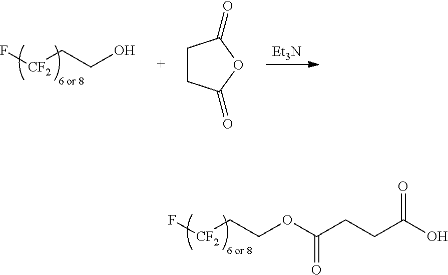

A Japanese patent publication of 1987 [JP01193336], which is incorporated herein by reference in its entirety, discloses an example method of synthesis and an example use of monoester, fluorinated derivatives of succinic acid in reaction with 1H,1H,2H,2H-heptadecafluoro-n-decanol or 1H,1H,2H,2H-tridecafluoro-n-octanol.

The first of these acids was used as an agent in a synthetic resin composition for use as a film of antifogging agent—tenside composition, e.g. applied onto glass surfaces and/or other functional surfaces. Another Japanese patent publication of 1998 [JP200155935], which is incorporated herein by reference in its entirety, discloses an example of the same compound used in a composition comprising magnetic data storage medium.

The same acid and its further derivative, namely a diester containing a single fragment derived from fluorinated alcohol and another fragment derived from fluorine-free alcohol, is disclosed in a Japanese patent publication of 2005 [JP2007070289], which is incorporated herein by reference in its entirety. This document discloses an example of properties of such diesters as surfactants and their use in compositions as gelling agents.

Another Japanese patent publication of 2012 [JP2013195630], which is incorporated herein by reference in its entirety, discloses an example of a use of asymmetric diesters of succinic acid in a composition of a liquid crystal, reflecting layer of glass, windows and car windshields, used as a filter of IR (infrared) radiation.

Another Japanese patent document of 2012 [JP2013241366], which is incorporated herein by reference in its entirety, discloses an example of an asymmetric diester derivative containing a fluorinated alcohol group on one hand and a sterol derivative of fluorine-free alcohol on the other. These compounds were used in a mixture as cosmetics in the form of hair spray. Examples of fluorinated derivatives of organic monoacids are also disclosed, one of which is a thioderivative of acetic acid, substituted at the sulphur atom with a fluorinated moiety.

Japanese patent publications [JP2003295407 and JP2002196459], which are both incorporated herein by reference in their entirety, disclose examples of alkaline metal salts (of lithium, sodium, potassium) of fluorinated acetic acid derivatives. These salts were used in material compositions used to obtain colour and/or greyscale photographic images.

An American patent publication of 1981 [U.S. Pat. No. 4,419,298], which is incorporated herein by reference in entirety, discloses examples of ammonium (diethanolammonium) salts with a fluorinated acetic acid derivative. This document also discloses an example of use of such compounds in a composition used to produce waterproof paper and textiles.

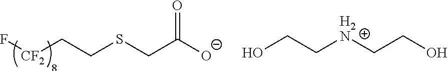

A 1995 publication [J. Fluorine Chemistry, 1995, 70, 19-26], which is incorporated herein by reference in its entirety, discloses examples of synthesis methods of 2H,2H-perfluoroalkyl- and 2H,2H-perfluoroalkenylcarboxylic acids and of their amides. A 2012 publication [J. Fluorine Chemistry, 2012, 135, 330-338], which is incorporated herein by reference in its entirety, discloses examples of synthesis methods of ethoxylated, fluorinated surfactants from perfluorinated carboxylic acids and their esters. Another, 2017 publication [ChemPhysChem 2017, 18, 1-13], which is incorporated herein by reference in entirety, discloses examples of synthesis methods and studied properties of perfluoroalkyldicarboxylic acids and their esters. A 2014 scientific paper [J. Fluorine Chemistry, 2014, 161, 60-65; DOI: 10.1016/j.jfluchem.2014.02.004], which is incorporated herein by reference in its entirety, discloses an example of a synthesis method and surface-active properties of new, hybrid surfactants. These surfactants contained two fragments, a fluorinated fragment in the anionic and a non-fluorinated fragment in the cationic part, as tertiary ammonium salts.

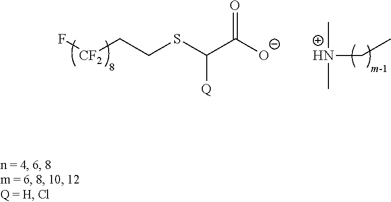

Patent [U.S. Pat. No. 4,423,061], which is incorporated herein by reference in its entirety, discloses examples of perfluorinated, organic cycloalkylamine derivatives used to produce emulsions, with excellent properties enabling solution and storage of large volumes of oxygen. Examples of production methods of ultrapermanent emulsions and foams are also disclosed in [EP1960097B1], which is incorporated herein by reference in its entirety.

Examples of ionic surfactants that are approved for use in medicines are disclosed in [R. C. Rowe; P. J. Sheskey; S. C. Owen, Handbook of Pharmaceutical Excipients 6th edition”, 2009], which is incorporated herein by reference in its entirety. Such ionic surfactants include: (i) benzalkonium chloride, (ii) benzethonium chloride, (iii) cetylpyridinium chloride, (iv) cetyltrimethylammonium bromide, (v) sodium bis(2-ethylhexyl) sulfosuccinate, (vi) sodium dodecylsulphate, (vii) phospholipid derivatives (phosphatidylcholine, phosphatidylglycerol, phosphatidylamines, etc.) and (viii) emulgating, anionic wax (containing cetostearyl alcohol derivatives). Ionic surfactants without perfluorinated chains result in less stable nanoemulsions or are not as efficient in their formation.

A demand for surfactants, the properties of which would enable their use in applications in biology, biochemistry and medicine for storage of tissues and sensitive biological materials still exists in the field.

The required, researched properties of these chemical compounds include adequate surface-active properties (surfactants with adequate emulsion stability and durability), high gas solubility, mainly solubility of oxygen and low surfactant cytotoxicity with relatively high concentrations of the compound in aqueous media. A significant problem related to most currently known surfactants used in biomedical applications lies in their highly cytotoxic properties.

Ammonium salts of partially fluorinated organic acids, the methods of synthesis thereof, and the uses and applications thereof may benefit from improvements.

### SUMMARY

It has been surprisingly found that optimisation of the exemplary structures of the obtained compounds leads to exemplary ammonium salts of fluorinated organic acids with the required surface-active properties and low cytotoxicity, enabling those surfactants to form the required oil-in water and/or water-in-oil emulsions.

An exemplary arrangement relates to a chemical compound comprising an ammonium salt of partially fluorinated organic acids, represented by an exemplary general Formula 1:

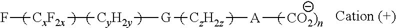

In exemplary arrangements:

CₓF₂ₓ—comprises a straight or a branched chain, where X=1 to 20;

C_{y}H_{2y}—comprises a straight or a branched chain, where Y=1 to 10;

C_{z} H_{2z}—comprises a straight or a branched chain, where Z=0 to 10;

G comprises one of a bond or a S, O atom or another heteroatom or a carbonyl group (CO), a carbonyloxy group (OCO);

A comprised one of a bond, —OCO—C_{z}H_{2z}—, where Z=0 to 10 or —OCO—Ar—, where Ar is benzene or naphthene, a —(C(H)—COOH)— group, a —(C(H)—COO—)— group;

n comprises 1 or 2;

Cation (+) comprises one of 1,1,3,3-tetramethylguanidinium cation, a lysinium cation, an argininium cation, a polylysinium cation. polycysteinium cation, or polytyrosine;

Or the cation is:

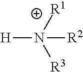

In exemplary arrangements, R¹, R², R³ are independently a hydrogen atom, an ethylenoxy group (—CH₂CH₂O—), a polyethylenoxy group ((—CH₂CH₂O-)n, wherein n is a natural number 1 to 10), a C₁-C₂₅ alkyl group, a C₁-C₂₅ alkoxy group, a C₃-C₁₂ cycloalkyl group, a C₁-C₅ perfluoroalkyl group, a C₂-C₁₂ alkenyl, a C₃-C₁₂ cycloalkenyl, a C₅-C₂₀ aryl, a C₅-C₂₄ aryloxy, a C₂-C₂₀ heterocyclic, a C₄-C₂₀ heteroaryl, a C₅-C₂₀ heteroaryloxy, a C₇-C₂₄ aralkyl, a C₅-C₂₄ perfluoroaryl, an —N(R′)(R″) amine group, substituted with hydrogen atoms or a halogen atom, or may be substituted with at least one C₁-C₁₂ alkyl group, C₁-C₁₂ perfluoroalkyl, C₁-C₁₂ alkoxy, C₅-C₂₄ aryloxy, C₂-C₂₀ heterocyclic, C₄-C₂₀ heteroaryl, C₅-C₂₀ heteroaryloxy, C₇-C₂₄ aralkil, C₅-C₂₄ perfluoroaryl, —N(R′)(R″) amine, —OR′ alkoxy group, wherein, in exemplary arrangements, R, R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl, C₅-C₂₀ aryl, C₅-C₂₄ aryloxy, C₂-C₂₀ heterocyclic, C₄-C₂₀ heteroaryl, C₅-C₂₀ heteroaryloxy group, which, may be connected, resulting in a substituted or unsubstituted C₄-C₁₀ cyclic or a C₄-C₁₂ polycyclic system, which may be substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy group.

In exemplary arrangements, substituents R¹ and R² or R² and R³ or R¹ and R³ or R¹, R² and R³ of the ammonium cation are connected, forming a chain or a ring system.

In exemplary arrangements, the ammonium cation comprises a tertiary cation, or a secondary cation or a primary cation.

In exemplary arrangements, the anion of a partially fluorinated carboxylic acid comprises an anion from the following list

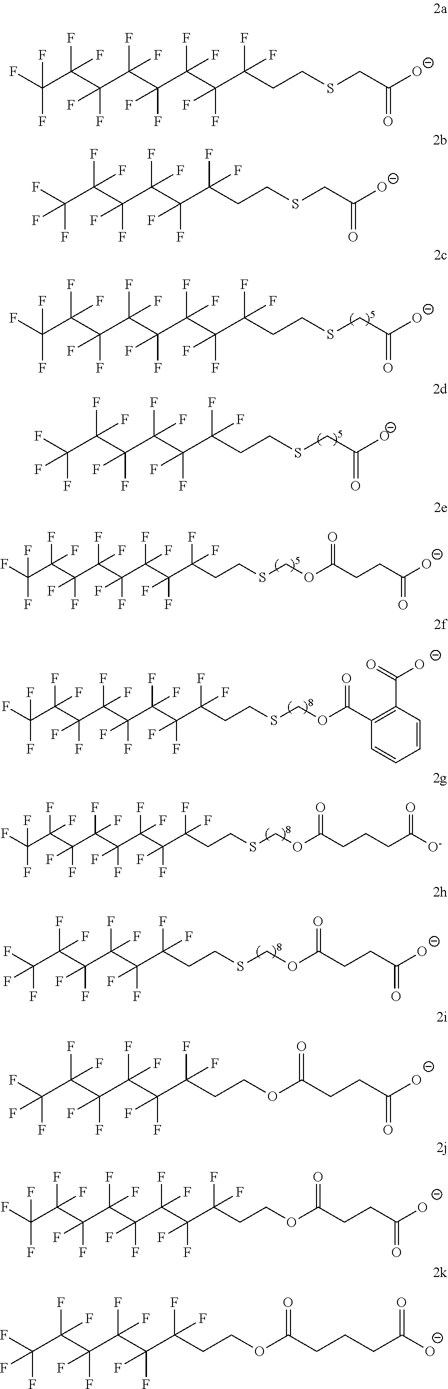

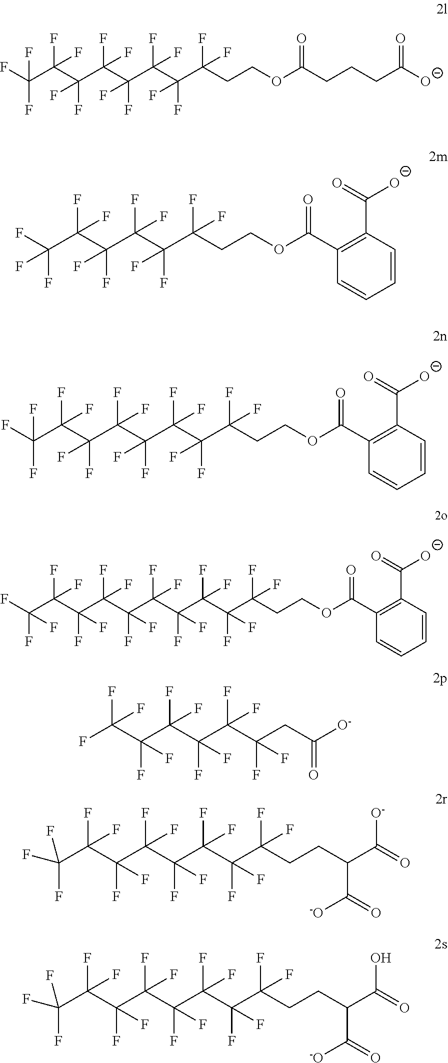

In exemplary arrangements, the ammonium cation comprises a cation from the following list

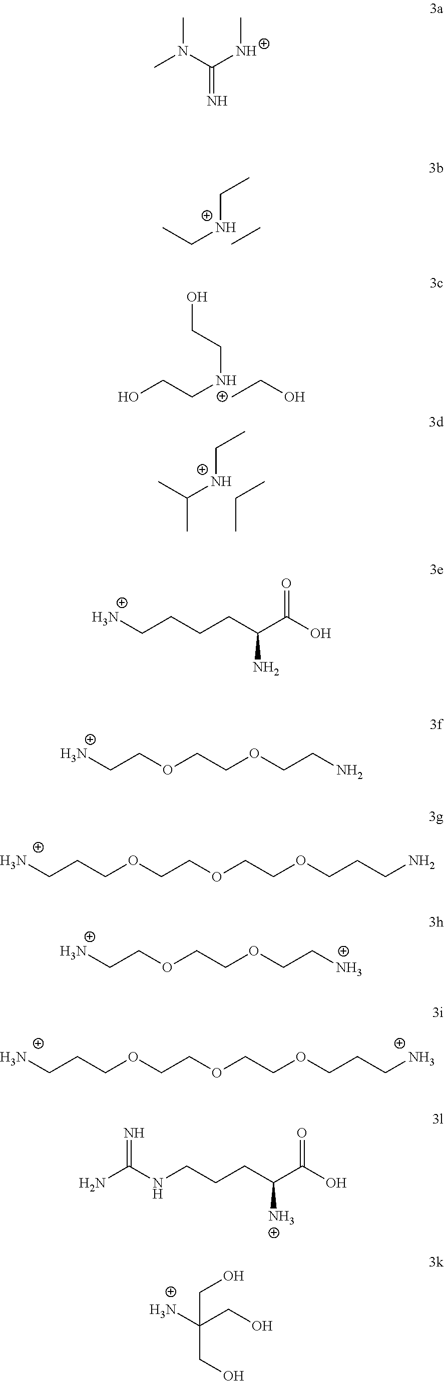

The exemplary arrangements also relate to use of the exemplary compound represented by Formula 1, comprising:

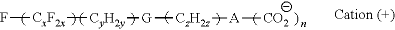

In exemplary arrangements:

CₓF₂ₓ—comprises a straight or a branched chain, where X=1 to 20;

C_{y}H_{2y}—comprises a straight or a branched chain, where Y=1 to 10;

C_{z}H_{2z}—comprises a straight or a branched chain, where Z=0 to 10;

G comprises one of a bond or a S, O atom, another heteroatom, a carbonyl group (CO) or, a carbonyloxy group (OCO);

A comprises one of a bond, —OCO—C_{z}H_{2z}— where Z=0 to 10, —OCO—Ar— where Ar is benzene or naphthene, a —(C(H)—COOH)— group, or a —(C(H)—COO—)— group;

n comprises 1 or 2;

Cation (+) comprises one of a 1,1,3,3-tetramethylguanidinium cation, a lysinium cation, an argininium cation, a polylysinium cation, a polycysteinium cation, a polytyrosinium cation, a potassium cation, or a sodium cation;

Or Cation (+) comprises:

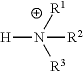

In exemplary arrangements, R¹, R², R³ are independently a hydrogen atom, an ethylenoxy group (—CH₂CH₂O—), a polyethylenoxy group ((—CH₂CH₂O-)n, wherein n is a natural number 1 to 10), a C₁-C₂₅ alkyl group, a C₁-C₂₅ alkoxy group, a C₃-C₁₂ cycloalkyl group, a C₁-C₅ perfluoroalkyl group, a C₂-C₁₂ alkenyl, a C₃-C₁₂ cycloalkenyl, a C₅-C₂₀ aryl, a C₅-C₂₄ aryloxy, a C₂-C₂₀ heterocyclic, a C₄-C₂₀ heteroaryl, a C₅-C₂₀ heteroaryloxy, a C₇-C₂₄ aralkyl, a C₅-C₂₄ perfluoroaryl, an —N(R′)(R″) amine group, substituted with hydrogen atoms or a halogen atom, or in exemplary arrangements, may be substituted with at least one C₁-C₁₂ alkyl group, C₁-C₁₂ perfluoroalkyl, C₁-C₁₂ alkoxy, C₅-C₂₄ aryloxy, C₂-C₂₀ heterocyclic, C₄-C₂₀ heteroaryl, C₅-C₂₀ heteroaryloxy, C₇-C₂₄ aralkil, C₅-C₂₄ perfluoroaryl, —N(R′)(R″) amine, —OR′ alkoxy group, wherein in exemplary arrangements, R, R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl, C₅-C₂₀ aryl, C₅-C₂₄ aryloxy, C₂-C₂₀ heterocyclic, C₄-C₂₀ heteroaryl, C₅-C₂₀ heteroaryloxy group, which may be connected, resulting in a substituted or unsubstituted C₄-C₁₀ cyclic or a C₄-C₁₂ polycyclic system, which may be substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy group, as a surfactant able to form water-in-oil and/or oil-in-water emulsions.

In exemplary arrangements, substituents R¹ and R² or R² and R³ or R¹ and R³ or R¹, R² and R³ of the ammonium cation are connected, forming a chain or a ring system.

In exemplary arrangements, the ammonium cation comprises a tertiary, a secondary, or a primary cation.

In exemplary arrangements, the anion of a partially fluorinated carboxylic acid comprises an anion from the following list,

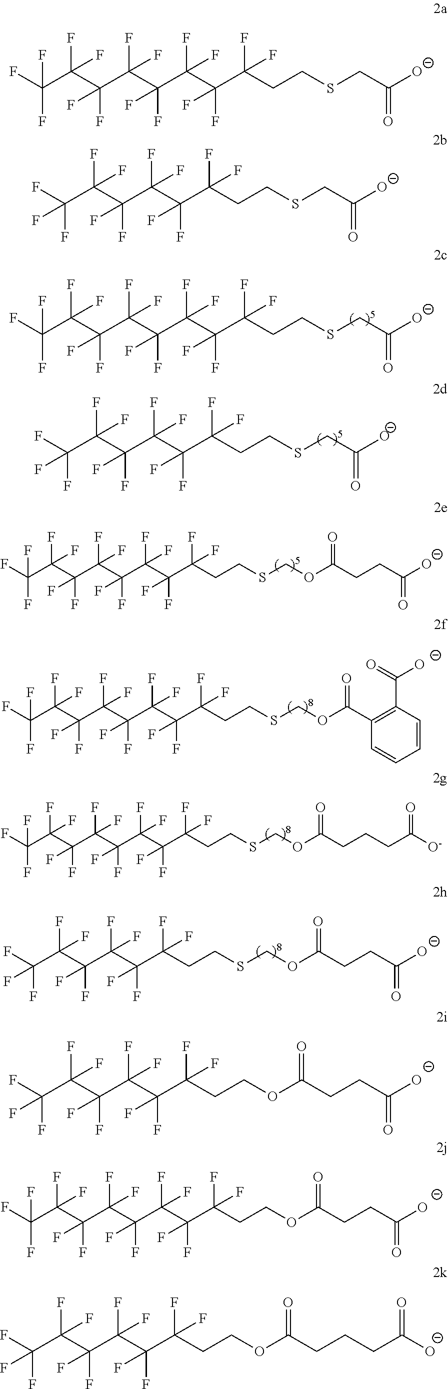

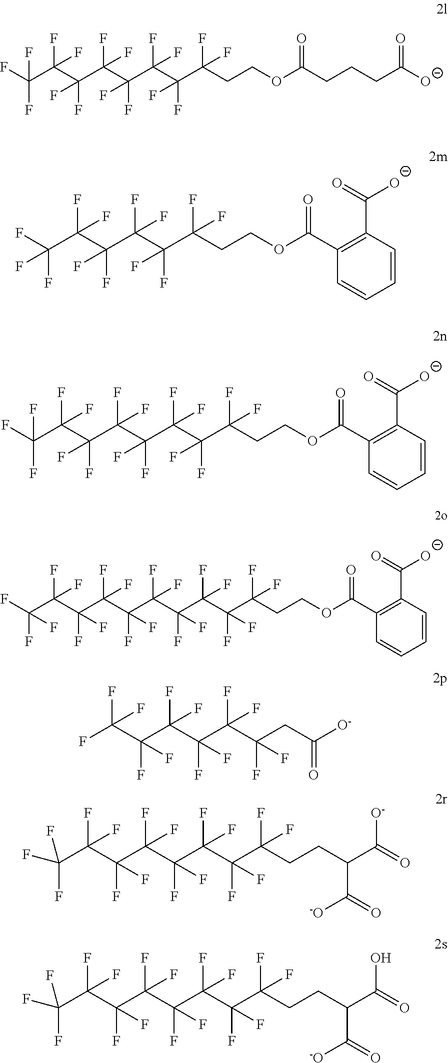

In exemplary arrangements, the ammonium cation comprises a cation from the following list

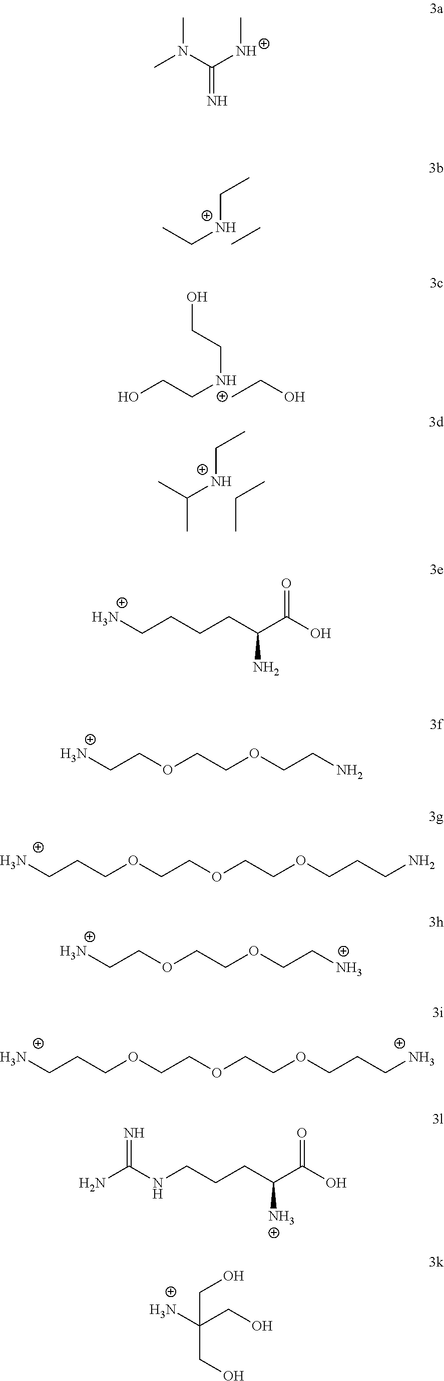

The exemplary arrangements also include use of the aforementioned exemplary compounds and methods in production of emulsions with high gas solubility, for example solubility of oxygen and/or air.

The exemplary arrangements also include use of the aforementioned exemplary compounds and methods in production of emulsions with emulsion particle sizes below 2 μm, for example 1.5 μm and 1 μm.

The exemplary arrangements also include use of the aforementioned exemplary compounds and methods in storage of organs, tissues, biological materials or extended medical storage.

The exemplary arrangements also include use of the aforementioned exemplary compounds and methods as a stabilising agent in blood substitute preparations.

The exemplary arrangements also include use of the aforementioned exemplary compounds and methods in therapy of stroke and in increasing the efficiency of photodynamic therapy of cancer.

The exemplary arrangements also include use of the aforementioned exemplary compounds and methods as a component of liquid enabling temporary support of breathing during artificial lung ventilation.

The exemplary arrangements also include use of the aforementioned exemplary compounds and methods as a component of liquids used in medical diagnostics, USG and MRI in particular.

The exemplary arrangements also include use of the aforementioned exemplary compounds and methods as a surfactant in compositions of medicinal drugs, vaccines and medical products.

The exemplary arrangements also include use of the aforementioned exemplary compounds and methods as a component of cosmetic, dermatological and care products.

The exemplary arrangements also include use of the aforementioned compounds and methods as a component of washing, cleaning and disinfecting agents.

The exemplary arrangements also include use of the aforementioned exemplary compounds and methods as a component of paints, dyeing emulsions, varnishes and plastics.

The exemplary arrangements also include use of the aforementioned exemplary compounds and methods as a component of agrochemical products.

The exemplary arrangements also include use of the aforementioned exemplary compounds and methods as a component of cooling mixtures in high-end computers and servers.

The exemplary arrangements also include use of the aforementioned exemplary compounds and methods as a component of culture medium delivering oxygen in bioreactors and other aerobic organism cultures.

The exemplary arrangements also include use of the aforementioned exemplary compounds and methods as a component of culture medium delivering carbon dioxide in bioreactors and other anaerobic organism cultures.

The exemplary arrangements also include use of the aforementioned exemplary compounds and methods in in vitro cultures of plant and animal cells.

The exemplary arrangements also include an exemplary method of synthesis of an exemplary ammonium salt of partially fluorinated organic acids, represented by the exemplary general Formula 1 comprising:

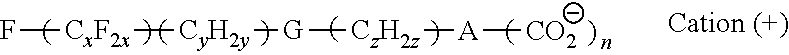

In exemplary arrangements:

CₓF₂ₓ—comprises a straight or a branched chain, where X=1 to 20;

C_{y}H_{2y}—comprises a straight or a branched chain, where Y=1 to 10;

C_{z}H_{2z}—comprises a straight or a branched chain, where Z=0 to 10;

G comprises one of a bond, a S atom, an O atom, another heteroatom, a carbonyl group (CO), or a carbonyloxy group (OCO);

A comprises one of a bond, a —OCO—C_{z}H_{2z}— where Z=0 to 10, a —OCO—Ar— group where Ar is benzene or naphthene, a —(C(H)—COOH)— group, or a —(C(H)—COO—)— group;

n comprises 1 or 2,

Cation (+) comprises 1 of a 1,1,3,3-tetramethylguanidinium cation, a lysinium cation, an argininium cation, a polylysinium cation, polycysteinium cation, polytyrosine, or the cation comprises:

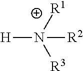

In exemplary arrangements, R¹, R², R³ are each independently a hydrogen atom, or alternatively comprise one of an ethylenoxy group (—CH₂CH₂O—), a polyethylenoxy group ((—CH₂CH₂O-)n, where n is a natural number 1 to 10), a C₁-C₂₅ alkyl group, a C₁-C₂₅ alkoxy group, a C₃-C₁₂ cycloalkyl group, a C₁-C₅ perfluoroalkyl group, a C₂-C₁₂ alkenyl, a C₃-C₁₂ cycloalkenyl, a C₅-C₂₀ aryl, a C₅-C₂₄ aryloxy, a C₂-C₂₀ heterocyclic, a C₄-C₂₀ heteroaryl, a C₅-C₂₀ heteroaryloxy, a C₇-C₂₄ aralkyl, a C₅-C₂₄ perfluoroaryl, an —N(R′)(R″) amine group, substituted with hydrogen atoms or a halogen atom, or in exemplary arrangements may be substituted with at least one C₁-C₁₂ alkyl group, C₁-C₁₂ perfluoroalkyl, C₁-C₁₂ alkoxy, C₅-C₂₄ aryloxy, C₂-C₂₀ heterocyclic, C₄-C₂₀ heteroaryl, C₅-C₂₀ heteroaryloxy, C₇-C₂₄ aralkil, C₅-C₂₄ perfluoroaryl, —N(R′)(R″) amine, —OR′ alkoxy group, wherein in exemplary arrangements R, R′ and R″ are the same or different C₁-C₂₅ alkyl group, C₃-C₁₂ cycloalkyl group, C₁-C₂₅ alkoxy group, C₂-C₂₅ alkenyl group, C₁-C₁₂ perfluoroalkyl, C₅-C₂₀ aryl, C₅-C₂₄ aryloxy, C₂-C₂₀ heterocyclic, C₄-C₂₀ heteroaryl, C₅-C₂₀ heteroaryloxy group, which may be connected, resulting in a substituted or unsubstituted C₄-C₁₀ cyclic or a C₄-C₁₂ polycyclic system, which may be substituted with at least one C₁-C₁₂ alkyl, C₁-C₁₂ perfluoroalkyl, C₁-C₁₂ alkoxy, C₅-C₂₄ aryloxy, C₂-C₂₀ heterocycle, C₄-C₂₀ heteroaryl, C₅-C₂₀ heteroaryloxy group, characterised in that the adequate, partially fluorinated organic acid represented with the exemplary general Formula 4, comprising:

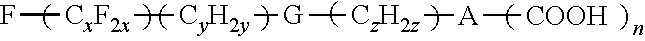

In the exemplary Formula 4, in which all the variables have the meanings specified above (in association with Formula 1), is subjected to a reaction with the adequate amine or amino acid, resulting in formation of an exemplary ammonium salt of partially fluorinated organic acids, represented with the general exemplary Formula 1.

In exemplary arrangements, the reaction is performed in a solvent, an alcohol, for example, in methanol or in a mixture of alcohol, with water.

In exemplary arrangements, the amine or amino acid is added to the respective acid dissolved in alcohol, for example, in methanol, in the form of pure alcohol or of an aqueous solution.

In exemplary arrangements, the respective acid is used as an ester.

In exemplary arrangements, the reaction mixture is heated, for example, to its boiling point.

### BRIEF DESCRIPTION OF DRAWINGS

FIG. 1 is a graph illustrating an example of the dependence between specific conductivity and surfactant concentration for exemplary compound 2a3e.

FIG. 2 is a graph illustrating the results of the XTT test for exemplary compound 2l3k.

FIG. 3 is a table illustrating microscopic images of L929 and HMEC-1 cell lines after 24-hour treatment with different concentration of exemplary compound 2l3k solutions.

FIG. 4 is a graph illustrating the results of the XTT test for exemplary compound 2a3g.

FIG. 5 is a table illustrating microscopic images of L929 and HMEC-1 cells after 24-hour treatment with exemplary compound 2a3g solutions at different concentrations.

FIG. 6 is a graph illustrating the result of—non hemolytic properties for exemplary compound 2j3k at concentrations 1% and lower.

FIG. 7 is a graph illustrating the result of—hemolytic properties for exemplary compound 2c3f at concentrations above 0.05%.

FIG. 8 is a table illustrating images of the histological visualization of a rat kidney after administration of analysed exemplary substances (HE staining, 4× lens).

FIG. 9 is a table illustrating images of the histological visualization of a rat liver after administration of analysed exemplary substances (HE staining, 4× lens).

FIG. 10 is a table illustrating images of the histological visualization of a rat heart after administration of analysed exemplary substances (HE staining, 4× lens).

FIG. 11 is a table illustrating images of the histological visualization of rat lungs after administration of analysed exemplary substances (HE staining, 4× lens).

FIG. 12 is a graph illustrating pH change in serum as a function of the number of administrations of the tested exemplary formulations.

FIG. 13 is a graph illustrating carbon dioxide partial pressure in blood change as a function of the number of administrations of the tested exemplary formulations.

FIG. 14 is a graph illustrating creatinine concentration in blood change as a function of the number of administrations of the tested exemplary formulations.

FIG. 15 is a graph illustrating lactate concentration in blood change as a function of the number of administrations of the tested exemplary formulations.

FIG. 16 is a graph illustrating lymphocyte concentration in blood change as a function of the number of administrations of the tested exemplary formulations.

FIG. 17 is a graph illustrating red blood cell concentration in blood change as a function of the number of administrations of the tested exemplary formulations.

FIG. 18 is a graph illustrating thrombocyte concentration in blood change as a function of the number of administrations of the tested exemplary formulations.

FIG. 19 is a graph of change in carbonate concentration in blood as a function of the number of administrations of the tested exemplary formulations.

FIG. 20 is a graph illustrating BE value change in extracellular fluid as a function of the number of administrations of the tested exemplary formulations.

FIG. 21 is a graph illustrating potassium ion concentration in blood change as a function of the number of administrations of the tested exemplary formulations.

FIG. 22 is a graph illustrating calcium ion concentration in blood change as a function of the number of administrations of the tested exemplary formulations.

FIG. 23 is a graph illustrating BE values in blood change as a function of the number of administrations of the tested exemplary formulations.

FIG. 24 is a graph illustrating anion gap value change as a function of the number of administrations of the tested exemplary formulations.

FIG. 25 is a graph illustrating systolic pressure measured on the rat's tail change as a function of the number of administrations of the tested exemplary formulations.

### DETAILED DESCRIPTION

Terms used in this disclosure have the meanings specified below. Undefined terms have their meanings as understood by a person having skill in the field, according to the state of the art knowledge, this disclosure, and the context of the Detailed Description, Summary and Background of this Application. Unless specified otherwise the following conventions of chemical terms are used in this disclosure, with meanings indicated in definitions presented below.

The term “halogen atom” or “halogen” indicates an element selected from F, Cl, Br, I.

The term “alkyl” refers to a saturated, linear or branched hydrocarbon substituent with the indicated number of carbon atoms. Example alkyl substituents include -methyl, -ethyl, -n-propyl, -n-butyl, -n-pentyl, -n-hexyl, -n-heptyl, -n-octyl, -n-nonyl and -n-decyl. Representative branched —(C₁-C₁₀) alkyls include -isopropyl, -sec-butyl, -isobutyl, -tert-butyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentyl, -3-methylpentyl, -4-methylpentyl, -1-ethylbutyl, -2-ethylbutyl, -3-ethylbutyl, -1,1-dimethylbutyl, -1,2-dimethylbutyl, -1,3-dimethylbutyl, -2,2-dimethylbutyl, -2,3-dimethylbutyl, -3,3-dimethylbutyl, -1-methylhexyl, -2-methylhexyl, -3-methylhexyl, -4-methylhexyl, -5-methylhexyl, -1,2-dimethylpentyl, -1,3-dimethylpentyl, -1,2-dimethylhexyl, -1,3-dimethylhexyl, -3,3-dimethylhexyl, -1,2-dimethylheptyl, -1,3-dimethylheptyl, -3,3-dimethylheptyl and similar substituents.

The term “alkoxy” refers to an alkyl substituent as specified above, connected via an oxygen atom.

The term “perfluoroalkyl” refers to an alkyl group as specified above, in which all hydrogen atoms have been substituted with identical or different halogen atoms.

The term “cycloalkyl” refers to a saturated, mono- or polycyclic hydrocarbon substituent with the indicated number of carbon atoms. Example cycloalkyl substituents include -cyclopropyl, -cyclobutyl, -cyclopentyl, -cyclohexyl, -cycloheptyl, -cyclooctyl, -cyclononyl, -cyclodecyl and similar groups.

The term “heteroatom” refers to an atom selected from a group including oxygen, sulphur, nitrogen, phosphorus and other atoms.

The term “NMR” refers to nuclear magnetic resonance.

The term “TMG” refers to tetramethylguanidyne.

The term “medical products” refers to the meaning of the term medical products as used in the technical field, as well as the dictionary definition including any product or material used to diagnose or manage patients.

The term “biological material” refers to the meaning of the term “biological material” as used in the technical field, as well as its ordinary dictionary definition including any naturally or artificially biocompatible materials that comprise a whole or a part of a living or artificial structure, or a biomedical device, material, or liquid that performs, augments or replaces or facilitates a natural function.

The term “DMAP” refers to 4-dimethylaminopirydne.

### EXAMPLES OF EXEMPLARY FORMULAS

The following examples are presented as an illustration of the exemplary formulas and explanation of their exemplary aspects only, and are not limiting and should not be identified with the entire scope of the exemplary arrangements, defined in the attached claims. Unless indicated otherwise, the following examples use standard materials and methods used in the fields or recommendations of manufacturers for specific reagents and methods.

### Example I

### Obtaining Organic Acids

TABLE 1
List of Obtained Acids

| | Exemplary Structural Formula | Symbol |
|---|---|---|
| 1. | 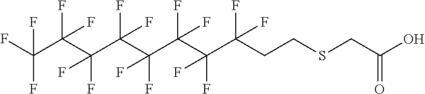 [SMILES: O=C(O)CSCCC(F)(F)C(F)(F)C(F)(F)C(F)(F)C(F)(F)C(F)(F)C(F)(F)C(F)(F)F] | 2a |
| 2. | 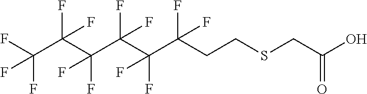 [SMILES: O=C(O)CSCCC(F)(F)C(F)(F)C(F)(F)C(F)(F)C(F)(F)C(F)(F)F] | 2b |
| 3. | 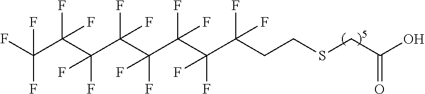 | 2c |
| 4. | 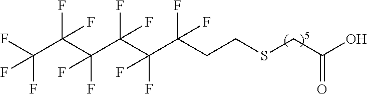 | 2d |
| 5. | 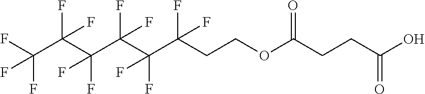 [SMILES: O=C(O)CCC(=O)OCCC(F)(F)C(F)(F)C(F)(F)C(F)(F)C(F)(F)C(F)(F)F] | 2i |
| 6. | 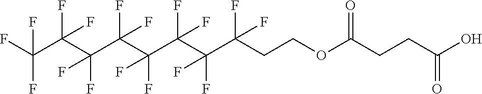 [SMILES: O=C(O)CCC(=O)OCCC(F)(F)C(F)(F)C(F)(F)C(F)(F)C(F)(F)C(F)(F)C(F)(F)C(F)(F)F] | 2j |
| 7. | 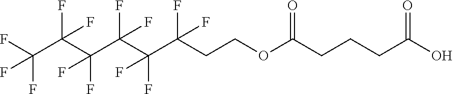 [SMILES: O=C(O)CCCC(=O)OCCC(F)(F)C(F)(F)C(F)(F)C(F)(F)C(F)(F)C(F)(F)F] | 2k |
| 8. | 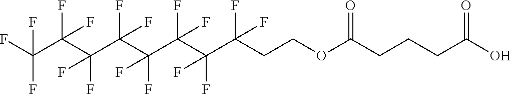 [SMILES: O=C(O)CCCC(=O)OCCC(F)(F)C(F)(F)C(F)(F)C(F)(F)C(F)(F)C(F)(F)C(F)(F)C(F)(F)F] | 2l |
| 9. | 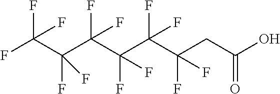 [SMILES: O=C(O)CC(F)(F)C(F)(F)C(F)(F)C(F)(F)C(F)(F)C(F)(F)F] | 2p |
| 10. | 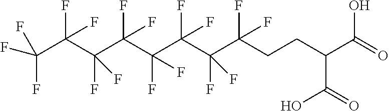 [SMILES: O=C(O)C(CCC(F)(F)C(F)(F)C(F)(F)C(F)(F)C(F)(F)C(F)(F)C(F)(F)C(F)(F)F)C(=O)O] | 2r |

### Exemplary Preparation of Acid 2a

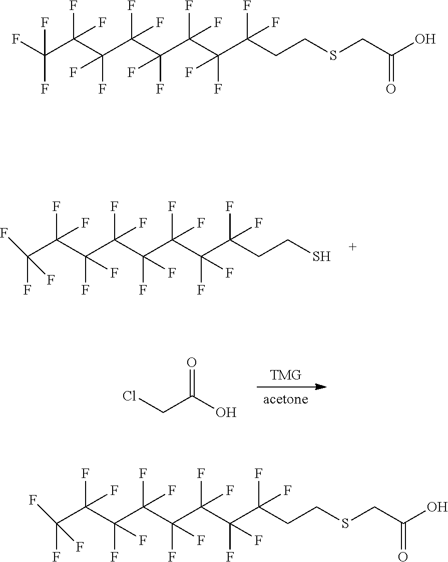 [SMILES: O=C(O)CSCCC(F)(F)C(F)(F)C(F)(F)C(F)(F)C(F)(F)C(F)(F)C(F)(F)C(F)(F)F ; FC(F)(F)C(F)(F)C(F)(F)C(F)(F)C(F)(F)C(F)(F)C(F)(F)C(F)(F)CCS ; O=C(O)CCl ; O=C(O)CSCCC(F)(F)C(F)(F)C(F)(F)C(F)(F)C(F)(F)C(F)(F)C(F)(F)C(F)(F)F]

In an exemplary method of preparation of the exemplary acid 2a, to a solution of 1H,1H,2H,2H-perfluorodecanethiol 0.480 g (1.00 mmol) in acetone (10 mL), 0.236 g (2.05 mmol) of TMG was added at room temperature, under an argon atmosphere. Then a solution of 0.095 g (1.00 mmol) chloroacetic acid in acetone (5 mL) was added dropwise. The reaction mixture was heated to 50° C. and stirred for 2 hours. Thereafter, the mixture was cooled to room temperature, the white precipitate was filtered off and washed with 10 mL of acetone. The filtrate was concentrated. Then 10 mL water was added, and mixture was acidified with a 1M solution of hydrochloric acid to pH=5-6 and was extracted with ethyl acetate (3×20 mL). The combined organic phases were washed twice with water and dried over MgSO₄. After concentration to dryness on an evaporator the crude product was purified by column chromatography using ethyl acetate-hexane mixtures (from 0 to 100% ethyl acetate) to obtain 3,3,4,4,5,5,6,6,7,7,8,8,8,9,10,10,10,10-heptadecafluoro-1-decathioacetic acid 2a (0.377 g, yield=70%).

### Spectral Analysis:

¹H NMR (500 MHz, acetone-d₆): δ=10.94 (s, 1H), 3.27 (s, 2H), 2.83 (dd, J=9.3, 6.7 Hz, 2H), 2.50 (ddd, J=26.5, 18.5, 8.1 Hz, 2H);

¹⁹F NMR (470 MHz, acetone-d₆): δ=−81.72 (dd, J=22.1, 11.6 Hz, 3F), −113.91-−114.14 (m, 2F), −114.58-−114.82 (m, 2F), −122.25 (s, 2F), −122.46 (s, 2F), −123.35 (d, J=61.9 Hz, 2F), −123.97 (d, J=55.9 Hz, 2F), −126.62-−126.96 (m, 2F);

¹³C NMR (126 MHz, acetone-d₆): δ=170.61, 32.77, 31.33, 22.79;

¹³C dec ¹⁹F NMR (126 MHz, acetone-d₆): δ=118.12, 117.02, 111.19, 111.02, 110.83, 110.76, 110.23, 108.40.

### Exemplary Preparation of Acid 2b

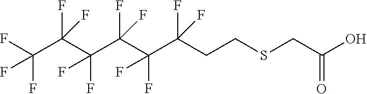 [SMILES: O=C(O)CSCCC(F)(F)C(F)(F)C(F)(F)C(F)(F)C(F)(F)C(F)(F)F]

In an exemplary method of preparation of the exemplary acid 2b, to a solution of 1H,1H,2H,2H-perfluorooctanothiol 0.380 g (1.00 mmol) in acetone (10 mL), 0.236 g (2.05 mmol) of TMG was added at room temperature, under an argon atmosphere. Then a solution of 0.095 g (1.00 mmol) chloroacetic acid in acetone (5 mL) was added dropwise. The reaction was carried out in accordance with the exemplary methodology to prepare the exemplary acid 2a to obtain 3,3,4,4,5,5,6,6,7,7,8,8,8-tridecafluoro-1-octanethioacetic acid 2b (0.402 g, yield=92%).

### Spectral Analysis:

¹H NMR (500 MHz, CD₃OD): δ=3.35-3.24 (m, 1H), 2.89 (dd, J=9.3, 6.8 Hz, 1H), 2.54 (ddd, J=26.2, 18.4, 8.1 Hz, 1H);

¹⁹F NMR (470 MHz, CD₃OD): δ=−82.40-−82.51 (m, 3F), −115.22-−115.59 (m, 2F), −122.96 (s, 2F), −123.93 (s, 2F), −124.31-−124.70 (m, 2F), −127.26-−127.45 (m, 2F);

¹³C NMR (126 MHz, CD₃OD): δ=172.41, 32.90, 31.22, 22.74;

¹³C dec ¹⁹F NMR (126 MHz, CD₃OD): δ=119.25, 118.56, 112.47, 112.35, 111.68, 109.89.

### Exemplary Preparation of Acid 2c

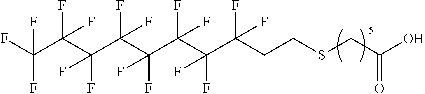

In an exemplary method of preparation of the exemplary acid 2c, to a solution of 1H,1H,2H,2H-perfluorodecanethiol 0.480 g (1.00 mmol) in acetone (10 mL), 0.236 g (2.05 mmol) of TMG was added at room temperature, under an argon atmosphere. Then a solution of 0.243 g (1.00 mmol) 6-bromohexanoic acid in acetone (5 mL) was added dropwise. The reaction was carried out in accordance with the exemplary methodology to prepare exemplary acid 2a to obtain 3,3,4,4,5,5,6,6,7,7,8,8,9,9,9,10,10,10-heptadecafluoro-1-decane-6-thiohexanoic acid 2c (0.534 g, yield=90%).

### Spectral Analysis:

¹H NMR (500 MHz, CD₃OD): δ=2.74 (dd, J=9.3, 6.8 Hz, 2H), 2.59 (t, J=7.3 Hz, 2H), 2.45 (ddd, J=26.3, 18.3, 8.3 Hz, 2H), 2.29 (t, J=7.4 Hz, 2H), 1.69-1.57 (m, 4H), 1.49-1.41 (m, 2H);

¹⁹F NMR (470 MHz, CD₃OD): δ=−82.42 (m, 3F), −115.07-−115.66 (m, 2F), −122.72 (m, 2F), −122.82-−123.09 (m, 4F), −123.60-−123.97 (m, 2F), −124.16-−124.68 (m, 2F), −127.17-−127.65 (m, 2F);

¹³C NMR (126 MHz, CD₃OD): δ=176.03, 33.32, 31.68, 31.30, 28.71, 27.82, 24.19, 21.86;

¹³C dec ¹⁹F NMR (126 MHz, CD₃OD): δ=119.28, 118.48, 112.58, 112.40, 112.21, 112.16, 111.63, 109.81.

### Exemplary Preparation of Acid 2d

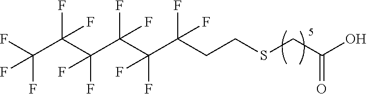

In an exemplary method of preparation of the exemplary acid 2d, to a solution of 1H,1H,2H,2H-perfluorooctanothiol 0.380 g (1.00 mmol) in acetone (10 mL), 0.236 g (2.05 mmol) of TMG was added at room temperature, under an argon atmosphere. Then a solution of 0.164 g (1.00 mmol) 8-chlorooctane-1-ol in acetone (5 mL) was added dropwise. The reaction was carried out in accordance with the exemplary methodology for preparation of exemplary acid 2a to obtain 3,3,4,4,5,5,6,6,7,7,8,8,8-tridecafluoro-1-octa-6-thiohexanoic acid 2d (0.412 g, yield=83%).

### Spectral Analysis:

¹H NMR (500 MHz, CD₃OD): δ=2.75 (dd, J=9.2, 6.8 Hz, 2H), 2.59 (t, J=7.3 Hz, 2H), 2.46 (ddd, J=26.1, 18.2, 8.0 Hz, 2H), 2.30 (t, J=7.4 Hz, 2H), 1.68-1.57 (m, 4H), 1.51-1.41 (m, 2H);

¹⁹F NMR (470 MHz, CD₃OD): δ=−82.46 (ddd, J=10.7, 6.1, 2.3 Hz, 3F), −115.26-−115.60 (m, 2F), −122.91 (d, J=56.0 Hz, 2F), −123.88 (d, J=56.1 Hz, 2F), −124.43 (d, J=14.2 Hz, 2F), −127.26-−127.63 (m, 2F);

¹³C NMR (126 MHz, CD₃OD): δ=176.04, 33.32, 31.67, 31.29, 28.71, 27.82, 24.19, 21.86;

¹³C dec ¹⁹F NMR (126 MHz, CD₃OD): δ=119.27, 118.56, 112.49, 112.37, 111.68, 109.89.

### Exemplary Preparation of Acid 2i

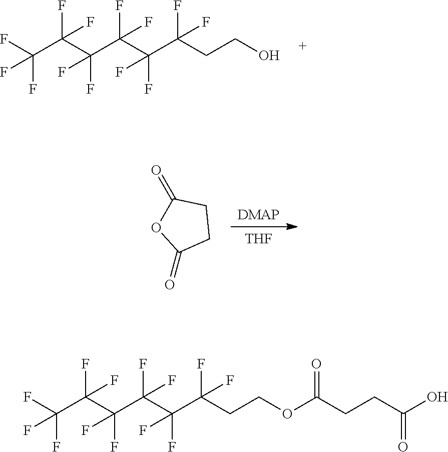 [SMILES: OCCC(F)(F)C(F)(F)C(F)(F)C(F)(F)C(F)(F)C(F)(F)F ; O=C1CCC(=O)O1 ; O=C(O)CCC(=O)OCCC(F)(F)C(F)(F)C(F)(F)C(F)(F)C(F)(F)C(F)(F)F ; "THF"]

In an exemplary method of preparation of the exemplary acid 2i, to a solution of 1H,1H,2H,2H-perfluoro-1-octanol (10 g, 27.46 mmol) in THF (3 mL), succinic anhydride (3.02 g, 30.21 mmol) and DMAP (0.67 g, 5.49 mmol) were added. The mixture was stirred at 100° C. for 2 hours. Thereafter, the mixture was cooled to 10° C. and 100 mL of water was added. The white precipitate was filtered off and washed with 10 mL of cold water. The precipitate was air dried to obtain 1H,1H,2H,2H-perfluoro-1-octyl succinic acid monoester 2i (12.33 g, yield=97%).

### Spectral Analysis:

¹H NMR (500 MHz, CDCl₃): δ=4.41 (t, J=6.5 Hz, 2H), 2.73-2.61 (m, 4H), 2.47 (tt, J=18.3, 6.5 Hz, 2H);

¹⁹F NMR (470 MHz, CDCl₃): δ=−80.87 (t, J=10.0 Hz, 3F), −113.42-−113.83 (m, 2F), −121.74-−122.11 (m, 2F), −122.78-−123.05 (m, 2F), −123.55-−123.84 (m, 2F), −126.02-−126.45 (m, 2F);

¹³C NMR (126 MHz, CDCl₃): δ=177.80, 171.63, 56.62, 30.43, 28.65, 28.61;

¹³C dec ¹⁹F NMR (126 MHz, CDCl₃): δ=117.39, 117.14, 110.95, 110.72, 110.18, 108.41.

### Exemplary Preparation of Acid 2j

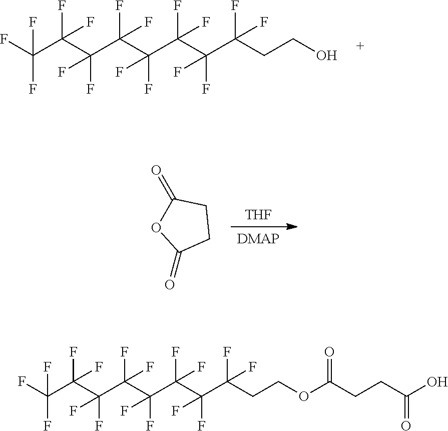

In an exemplary method of preparation of exemplary acid 2j, to a solution of 1H,1H,2H,2H-perfluoro-1-decanol (15 g, 32.32 mmol) in THF (15 mL), succinic anhydride (3.43 g, 34.26 mmol) and DMAP (0.39 g, 3.23 mmol) were added. The mixture was stirred at 100° C. for 2 hours. Thereafter, the mixture was cooled to 10° C. and 100 mL of water was added. The white precipitate was filtered off and washed with 10 mL of cold water. The precipitate was air dried to obtain 1H,1H,2H,2H-perfluoro-1-decyl succinic acid monoester 2j (17.81 g, yield=98%).

### Spectral Analysis:

¹H NMR (500 MHz, aceton-d₆): δ=4.43 (t, J=6.0 Hz, 2H), 2.74-2.56 (m, 6H);

¹⁹F NMR (470 MHz, aceton-d₆): δ=−81.35-−81.92 (m, 3F), −113.96-−114.32 (m, 2F), −122.23 (d, J=9.3 Hz, 2F), −122.34-−122.59 (m, 4F), −123.29 (s, 2F), −124.15 (s, 2F), −126.68-−126.82 (m, 2F);

¹³C NMR (126 MHz, aceton-d₆): δ=172.59, 171.69, 56.04, 30.02, 28.60, 28.09;

¹³C dec ¹⁹F NMR (126 MHz, CDCl₃): δ=118.06, 117.02, 111.15, 110.87, 110.78, 110.72, 110.19, 108.36.

### Exemplary Preparation of Acid 2k

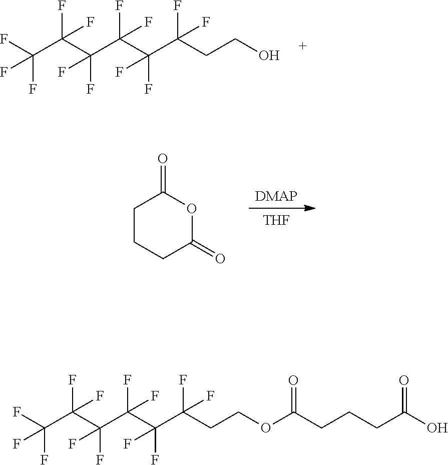 [SMILES: OCCC(F)(F)C(F)(F)C(F)(F)C(F)(F)C(F)(F)C(F)(F)F ; O=C1CCCC(=O)O1 ; O=C(O)CCCC(=O)OCCC(F)(F)C(F)(F)C(F)(F)C(F)(F)C(F)(F)C(F)(F)F ; "THF"]

In an exemplary method of preparation of exemplary acid 2k, to a solution of 1H,1H,2H,2H-perfluoro-1-octanol (5.18 g, 14.23 mmol) in THF (6 mL), glutaric anhydride (1.78 g, 15.65 mmol) and DMAP (0.35 g, 2.85 mmol) were added. The mixture was stirred at 100° C. for 4 hours. Thereafter, the mixture was cooled to 10° C. and 100 mL of water was added. The white precipitate was filtered off and washed with 10 mL cold water. The precipitate was dissolved in ethyl acetate and a saturated NaHCO₃ solution was added. The aqueous phase was separated and HCl was added to aqueous to pH-6. The aqueous layer was extracted three times with ethyl acetate. The organic fractions were combined, washed with brine, dried with MgSO₄ and concentrated to obtain 1H,1H,2H,2H-perfluoro-1-octyl glutaric acid monoester 2k (5.78 g, yield=85%).

### Spectral Analysis:

¹H NMR (500 MHz, acetone-d₆): δ=4.43 (t, J=6.2 Hz, 2H), 2.74-2.61 (m, 2H), 2.43 (t, J=7.4 Hz, 2H), 2.38 (t, J=7.3 Hz, 2H), 1.95-1.85 (m, 2H);

¹⁹F NMR (470 MHz, acetone-d₆): δ=−80.75-−83.66 (m, 3F), −114.02-−114.22 (m, 2F), −122.35-−122.63 (m, 2F), −123.44-−123.60 (m, 2F), −124.14-−124.35 (m, 2F), −126.79-−126.98 (m, 2F);

¹³C NMR (126 MHz, acetone-d₆): δ=173.25, 172.11, 55.87, 32.58, 32.44, 30.02, 19.87;

¹³C dec ¹⁹F NMR (126 MHz, acetone-d₆): δ=118.13, 117.15, 111.10, 110.89, 110.28, 108.49.

### Exemplary Preparation of Acid 2l

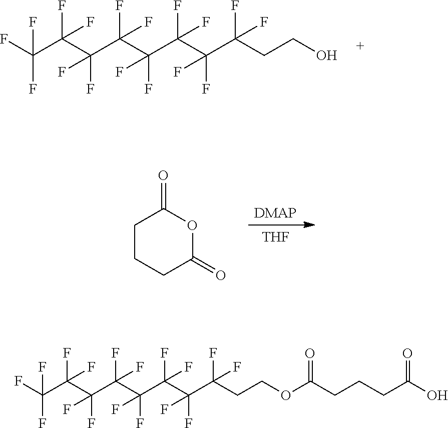

In an exemplary method of preparation of the exemplary acid 2l, to a solution of 1H,1H,2H,2H-perfluoro-1-decanol (3.70 g, 7.97 mmol) in THF (5 mL), glutaric anhydride (1.00 g, 8.77 mmol) and DMAP (0.19 g, 1.59 mmol) were added. The mixture was stirred at 100° C. for 4 hours. Thereafter, the mixture was cooled to 10° C. and 70 mL of water was added. The white precipitate was filtered off and washed with 10 mL of cold water. The precipitate was air dried to obtain 1H,1H,2H,2H-perfluoro-1-decyl glutaric acid monoester 2l (3.82 g, yield=83%).

### Spectral Analysis:

¹H NMR (500 MHz, acetone-d₆): δ=4.43 (t, J=6.2 Hz, 2H), 2.73-2.62 (m, 2H), 2.43 (t, J=7.4 Hz, 2H), 2.38 (t, J=7.3 Hz, 2H), 1.90 (dp, J=22.1, 7.4 Hz, 2H);

¹⁹F NMR (470 MHz, acetone-d₆): δ=−81.47-−82.04 (m), −113.86-−114.23 (m), −122.26 (s, J=56, 9 Hz), −122.47 (s), −122.49 (s), −123.30 (s), −124.16 (s), −126.66-−126.85 (m);

¹³C NMR (126 MHz, acetone-d₆): δ=173.18, 172.11, 55.88, 32.59, 32.13, 30.03, 19.89.

### Exemplary Preparation of Acid 2p

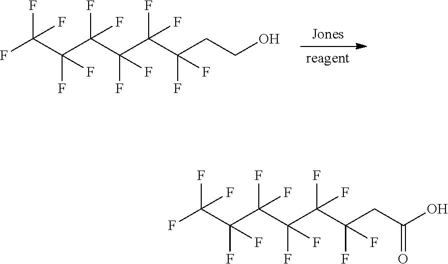 [SMILES: OCCC(F)(F)C(F)(F)C(F)(F)C(F)(F)C(F)(F)C(F)(F)F ; "reagent" ; "Jones" ; O=C(O)CC(F)(F)C(F)(F)C(F)(F)C(F)(F)C(F)(F)C(F)(F)F]

In an exemplary method of preparation of exemplary acid 2p, to a solution of 1H,1H,2H,2H-perfluoro-1-octanol (3.56 g, 3.78 mmol) in acetone (5 mL) Jones reagent was added dropwise. The reaction was terminated when the solution turned solid yellow with simultaneous precipitation of green chromium salts. Then 2-propanol was added dropwise to reduce the excess oxidizing reagent. The blue solid was decanted, 50 mL water was added to the residue. The product was extracted with diethyl ether (4×50 mL). The organic layers were combined and washed with water (50 mL), dried over MgSO₄ and concentrated under reduced pressure. The product was purified by recrystallization from CHCl₃ to give white crystals 2H,2H-perfluorooctanoic acid 2p (3.10 g, yield=84%).

### Spectral Analysis:

¹H NMR (500 MHz, acetone-d₆): δ=3.41 (t, J=18.5 Hz, 2H);

¹⁹F NMR (470 MHz, acetone-d₆): δ=−81.73-−81.85 (m, 3F), −112.36-−122.74 (m, 2F), −122.34-−122.62 (m, 2F), −123.36-−123.80 (m, 4F), −126.73-−126.99 (m, 2F);

¹³C NMR (126 MHz, acetone-d₆): δ=164.36 (s), 35.90 (t, J=22.0 Hz).

### Exemplary Preparation of Acid 2r

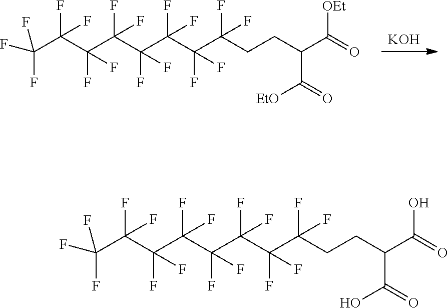

In an exemplary method of preparation of the exemplary acid 2r, to a solution of diethyl 2-(1H,1H,2H,2H-perfluorodecyl) malonate (9.66 g, 15.9 mmol) in ethanol (32 mL) solution of KOH (2.68 g, 47.7 mmol) in water (4 mL) was added and stirred overnight at reflux (110° C.). The suspension was diluted with water (50 mL) and washed with diethyl ether (3 x 50 mL). The aqueous phase was cooled to 0° C. and acidified using concentrated HCl (37%) until pH <2. The aqueous phase was then extracted with diethyl ether (3×70 mL). The organic layers were combined, dried over MgSO₄ and concentrated under reduced pressure. The product was purified by column chromatography starting from 10% ethyl acetate and 90% hexane, and then using eluents with a concentration gradient from 10% -100% ethyl acetate to obtain2-(1H,1H,2H,2H-perfluorodecyl) malonic acid 2r (5.94 g, yield=68%).

### Spectral Analysis:

¹H NMR (500 MHz, acetone-d₆): δ=3.61 (t, J=7.1 Hz, 1H), 2.42 (tt, J=18.7, 8.1 Hz, 2H), 2.22-2.14 (m, 2H);

¹⁹F NMR (470 MHz, acetone-d₆): δ=−81.73 (t, J=10.1 Hz, 3F), −115.08 (tq, J=32.8, 18.5 Hz, 2F), −122.27 (dd, J=22.2, 10.6 Hz, 2F), −122.48 (qt, J=18.9, 10.0, 9.5 Hz, 4F), −123.17-−123.45 (m, 2F), −124.08 (q, J=17.0, 16.2 Hz, 2F), −126.78 (qd, J=11.7, 9.8, 4.6 Hz, 2F);

¹³C NMR (126 MHz, acetone-d₆): δ=170.31, 50.71, 29.19, 20.67.

### Example II

### Preparation of Ammonium Salts of Partially Fluorinated Carboxylic Acids

### Exemplary Preparation of the Salt 2a3a

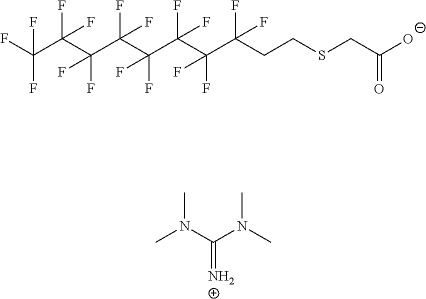

In an exemplary method of preparation of the exemplary salt 2a3a, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.115 g (1.00 mmol) of TMG was added. After refluxing for 2 hours the mixture was cooled and concentrated to dryness to give the product 0.653 g (yield=100%).

### Spectral Analysis:

¹H NMR (500 MHz, CD₃OD): δ=3.17 (s, 2H), 2.98 (s, 8H), 2.87-2.76 (m, 2H), 2.53 (ddd, J=26.5, 18.5, 8.2 Hz, 2H);

¹⁹F NMR (470 MHz, CD₃OD): δ=−81.57-−83.84 (m, 3F), −113.41-−117.50 (m, 2F), −122.26-−122.80 (m, 2F), −122.82-−123.28 (m, 4F), −123.51-−124.12 (m, 2F), −124.16-−124.83 (m, 2F), −126.80-−127.76 (m, 2F);

¹³C NMR (126 MHz, CD₃OD): δ=227.82, 226.21, 215.96, 177.36, 163.28, 39.90, 38.17, 32.98, 32.84, 32.60, 24.01.

### Exemplary Preparation of the Salt 2a3b

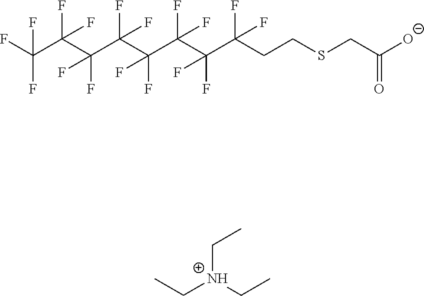

In an exemplary method of preparation of the exemplary salt 2a3b, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.101 g (1.00 mmol) of triethylamine was added. After refluxing for 2 hours, the mixture was cooled and concentrated to dryness to give the product 0.630 g (yield=98%).

### Spectral Analysis:

¹H NMR (500 MHz, CD₃OD): δ=3.30 (dt, J=3.2, 1.6 Hz, 1H), 3.23 (s, 2H), 3.20 (q, J=7, 3 Hz, 6H), 2.86 (dd, J=9.4, 6.8 Hz, 2H), 2.61-2.46 (m, 2H), 1.30 (t, J=7.3 Hz, 9H);

¹⁹F NMR (470 MHz, CD₃OD): δ=−79.75-−84.68 (m, 4F), −114.93-−115.58 (m, 2F), −122.46-−122.81 (m, 2F), −122.85-−123.15 (m, 4F), −123.76 (s, 2F), −124.13-124.76 (m, 2F), −126.98-−127.67 (m, 2F);

¹³C NMR (126 MHz, CD₃OD): δ=175.94, 47.78, 36.63, 33.12, 24.38, 19.37.

### Exemplary Preparation of the Salt 2a3c

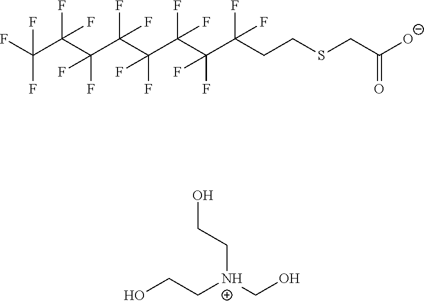 [SMILES: O=C([O-])CSCCC(F)(F)C(F)(F)C(F)(F)C(F)(F)C(F)(F)C(F)(F)C(F)(F)C(F)(F)F ; OCC[NH+](CO)CCO]

In an exemplary method of preparation of the exemplary salt 2a3c, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.149 g (1.00 mmol) of triethanolamine was added. After refluxing for 2 hours, the mixture was cooled and concentrated to dryness to give the product 0.685 g (yield=99%).

### Spectral Analysis:

¹H NMR (500 MHz, CD₃OD): δ=3.82 (t, J=10.6 Hz, 6H), 3.27-3.22 t, J=10.6 Hz, 6H), 3.21 (s, 2H), 2.84 (dd, J=9.4, 6.8 Hz, 2H), 2.53 (ddd, J=26.5, 18.4, 8.2 Hz, 2H);

¹⁹F NMR (470 MHz, CD₃OD): δ=−82.40 (t, J=10.2 Hz, 3F), −114.90-−115.91 (m, 2F), −122.58-−122.82 (m, J=8.7 Hz, 2F), −122.83-−123.06 (m, 4F), −123.65-−123.91 (m, 2F), −124.25-−124.53 (m, 2F), −127.20-−127.46 (m, 2F);

¹³C NMR (126 MHz, CD₃OD): δ=175.30, 56.21, 55.82, 35.90, 31.35, 22.62.

### Exemplary Preparation of the Salt 2a3d

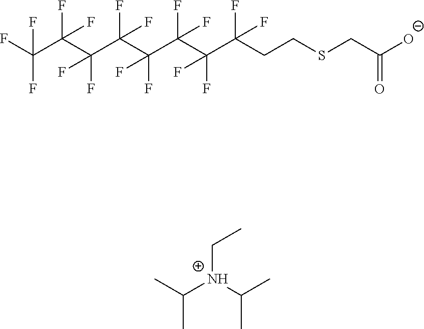

In an exemplary method of preparation of the exemplary salt 2a3d, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.129 g (1.00 mmol) of diisopropylethylamine was added. After refluxing for 2 hours the mixture was cooled and concentrated to dryness to give the product 0.660 g (yield=98%).

### Spectral Analysis:

¹H NMR (500 MHz, CD₃OD): δ=3.72 (dp, J=13.3, 6.6 Hz, 2H), 3.30 (s, 1H), 3.22 (q, J=7.4 Hz, 2H), 3.11 (ddd, J=13.5, 10.4, 5.4 Hz, 2H), 2.82-2.56 (m, 2H), 1.37 (d, J=6.3 Hz, 6H), 1.35-1.26 (m, 3H);

¹⁹F NMR (470 MHz, CD₃OD): δ=−80.02-−83.37 (m, 3F), −114.55 (dd, J=59.0, 43.3 Hz, 2F), −122, 49-−122.76 (m, 2F), −122.77-−123.10 (m, 4F), −123.58-−123.94 (m, 2F), −124.31 (d, J=103.2 Hz, 2F), −127.07-−127.50 (m, 2F);

¹³C NMR (126 MHz, CD₃OD): δ=168.16, 54.19, 42.69, 41.93, 23.71, 17.43, 15.84, 11.09.

### Exemplary Preparation of the Salt 2a3e

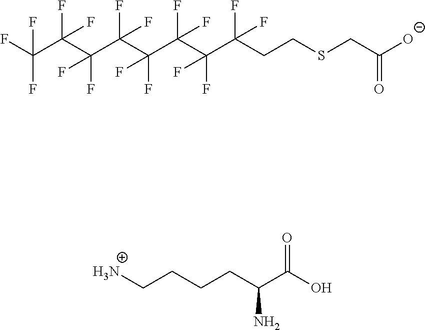 [SMILES: O=C([O-])CSCCC(F)(F)C(F)(F)C(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2a3e, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and a solution of 0.146 g (1.00 mmol) L-lysine in water (0.5 mL) was added. To the reaction mixture, 5 mL of water was added and the mixture was refluxed for 2 hours, followed by cooling and concentrating to dryness to give the product 0.684 g (yield=100%).

### Spectral Analysis:

¹H NMR (500 MHz, D₂O/CD₃CN): δ=3.90 (t, J=6.1 Hz, 1H), 3.48 (s, 2H), 3.19 (t, J=7.5 Hz, 2H), 3.03-2.95 (m, 2H), 2.65 (td, J=18.9, 8.9 Hz, 2H), 2.24 (dt, J=4.9, 2.5 Hz, 1H), 2.17-1.99 (m, 2H), 1.96-1.84 (m, 2H), 1.75-1.57 (m, 2H), 1.38 (dd, J=46.7, 19.6 Hz, 2H);

¹⁹F NMR (470 MHz, D₂O/CD₃CN): δ=−81.29-−83.80 (m, 3F), −114.11-−115.71 (m, 2F), −122.64 (s, 2F), −122.90 (s, 4F), −123.05 (s, 2F), −123.78 (d, J=206.9 Hz, 2F), −127.65 (s, 2F);

¹³C NMR (126 MHz, D₂O/CD₃CN): δ=176.45, 174.49, 54.59, 39.14, 37.33, 31.03, 30.04, 26.48, 22.68, 21.57.

### Exemplary Preparation of the Salt 2a3f

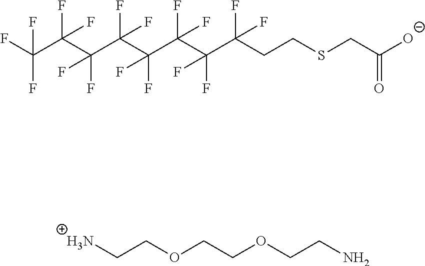 [SMILES: O=C([O-])CSCCC(F)(F)C(F)(F)C(F)(F)C(F)(F)C(F)(F)C(F)(F)C(F)(F)C(F)(F)F ; NCCOCCOCC[NH3+]]

In an exemplary method of preparation of the exemplary salt 2a3f, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.148 g (1.00 mmol) of 2.2′-(ethylenedioxy)-bis(ethylamine) was added. After refluxing for 2 hours the mixture was cooled and concentrated to dryness to give the product 0.680 g (yield=98%).

### Spectral Analysis:

¹H NMR (500 MHz, CD₃OD): δ=3.66 (d, J=9.4 Hz, 4H), 3.65-3.57 (m, 4H), 3.18 (s, 2H), 2.97 (dd, J=14.8, 9.4 Hz, 4H), 2.82 (dd, J=9.4, 6.8 Hz, 2H), 2.53 (ddd, J=26.5, 18.3, 8.2 Hz, 2H), 1.91 (d, J=24.4 Hz, 4H);

¹⁹F NMR (470 MHz, CD₃OD): δ=−82.39 (t, J=10.1 Hz, 3F), −114.82-−116.17 (m, 2F), −122.58-−122.81 (m, 2F), −122.90 (s, J=7.4 Hz, 4F), −123.75 (s, 2F), −124.37 (s, J=80.6 Hz, 2F), −126.87-−128.01 (m, 2F);

¹³C NMR (126 MHz, CD₃OD): δ=176.03, 69.89, 69.19, 39.85, 36.71, 31.38, 22.57.

### Exemplary Preparation of the Salt 2a3g

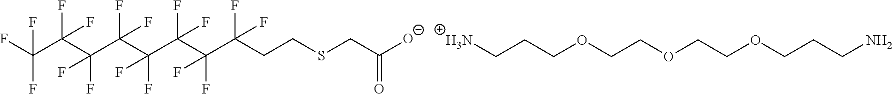 [SMILES: O=C([O-])CSCCC(F)(F)C(F)(F)C(F)(F)C(F)(F)C(F)(F)C(F)(F)C(F)(F)C(F)(F)F ; NCCCOCCOCCOCCC[NH3+]]

In an exemplary method of preparation of the exemplary salt 2a3g, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.220 g (1.00 mmol) of 4,7,10-trioxo-1,13-tridecanediamine was added. After refluxing for 2 hours the mixture was cooled and concentrated to dryness to give the product 0.735 g (yield=99%).

### Spectral Analysis:

¹H NMR (500 MHz, CD₃OD): δ=3.66 (d, J=9.4 Hz, 4H), 3.65-3.57 (m, 4H), 3.18 (s, 2H), 2.97 (dd, J=14.8, 9.4 Hz, 4H), 2.82 (dd, J=9.4, 6.8 Hz, 2H), 2.53 (ddd, J=26.5, 18.3, 8.2 Hz, 2H), 1.91 (d, J=24.4 Hz, 4H);

¹⁹F NMR (470 MHz, CD₃OD): δ=−80.47-−83.64 (m, 3F), −114.25-−115.96 (m, 2F), −122.70 (s, 2F), −122.78-−123.25 (m, 4F), −123.75 (s, 2F), −124.38 (s, 2F), −126.89-−127.50 (m, 2F);

¹³C NMR (126 MHz, CD₃OD): δ=176.00, 69.89, 69.66, 68.83, 38.42, 36.72, 31.39, 29.22, 22.57.

### Exemplary Preparation of the Salt 2a3l

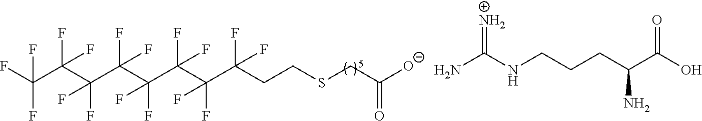

In an exemplary method of preparation of the exemplary salt 2a3l, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and a solution of 0.174 g (1.00 mmol) L-arginine in water (1 mL) was added. After refluxing for 2 hours the mixture was cooled and concentrated to dryness to give the product 0.696 g (yield=98%).

### Spectral Analysis:

¹H NMR (500 MHz, D₂O/CD₃CN): δ=3.82-3.67 (m, 1H), 3.65-3.55 (m, 2H), 3.20 (t, J=6.6 Hz, 2H), 2.81-2.65 (m, 2H), 2.58 (t, J=7.2 Hz, 2H), 2.34 (s, 2H), 2.23-2.10 (m, 2H), 2.04 (dt, J=4.9, 2.5 Hz, 2H), 1.98-1.76 (m, 4H), 1.75-1.48 (m, 4H), 1.46-1.30 (m, 2H);

¹⁹F NMR (470 MHz, D₂O/CD₃CN): δ=−83.21 (s, 3F), −113.12-−114.68 (m, 2F), −115.40 (s, 2F), −122.83 (s, 2F), −123.07 (s, 2F), −123.20 (s, 2F), −124.16 (s, 2F), −127.89 (s, 2F);

¹³C NMR (126 MHz, D₂O/CD₃CN): δ=176.56, 174.41, 156.92, 54.36, 40.63, 37.38, 31.05, 27.77, 24.11, 22.68.

### Exemplary Preparation of the Salt 2aK

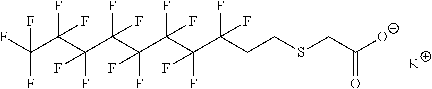 [SMILES: O=C([O-])CSCCC(F)(F)C(F)(F)C(F)(F)C(F)(F)C(F)(F)C(F)(F)C(F)(F)C(F)(F)F ; [K+]]

In an exemplary method of preparation of the exemplary salt 2aK, 3,3,4,4,5,5,6,6,7,7,8,8,9,9,10,10,10-heptadecafluoro-1-decanothioacetic acid 2a 0.538 g (1.00 mmol) was dissolved in 20 mL of methanol and 0.056 g (1.00 mmol) of potassium hydroxide was added. After refluxing for 2 hours the mixture was cooled and concentrated to dryness to give the product 0.480 g (yield=91%).

### Spectral Analysis:

¹H NMR (500 MHz, D₂O): δ=3.28 (s, 2H), 2.78 (dd, J=22.0, 13.9 Hz, 2H), 2.44 (ddd, J=26.5, 18.2, 8.1 Hz, 2H);

¹⁹F NMR (470 MHz, D₂O): δ=−80.45-−84.64 (m, 3F), −113.52-−116.82 (m, 2F), −122.55 (d, 2F), −123.00 (s, 4F), −123.16 (s, 2F), −124.10 (s, 2F), −127.36-−128.25 (m, 2F);

¹³C NMR (126 MHz, D₂O): δ=176.61, 37.41, 30.97, 22.64.

### Exemplary Preparation of the Salt 2a3c

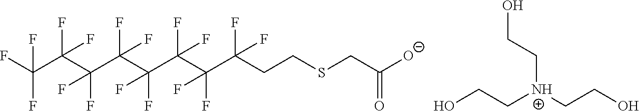 [SMILES: O=C([O-])CSCCC(F)(F)C(F)(F)C(F)(F)C(F)(F)C(F)(F)C(F)(F)C(F)(F)C(F)(F)F ; OCC[NH+](CCO)CCO]

In an exemplary method of preparation of the exemplary salt 2a3c, 3,3,4,4,5,5,6,6,7,7,8,8,8-tridecafluoro-1-octanethioacetic acid 2b 0.438 g (1.00 mmol) was dissolved in 20 mL of methanol and 0.149 g (1.00 mmol) of triethanolamine was added. After refluxing for 2 hours the mixture was cooled and concentrated to dryness to give the product 0.570 g (yield=97%).

### Spectral Analysis:

¹H NMR (500 MHz, CD₃OD): δ=3.95-3.88 (m, 3H), 3.39 (dd, J=14.4, 9.4 Hz, 3H), 3.22 (s, 1H), 2.83-2.76 (m, 1H), 2.48 (ddd, J=26.2, 18.0, 7.8 Hz, 1H);

¹⁹F NMR (470 MHz, CD₃OD): δ=−82.19 (t, J=10.1 Hz, 3F), −114.70-−115.53 (m, 2F), −122.85 (s, 2F), −123.83 (s, 2F), −124.25 (s, 2F), −127.21 (dd, J=14.4, 8.3 Hz, 2F);

¹³C NMR (126 MHz, CD₃OD): δ=177.67, 56.55, 56.49, 37.91, 32.17, 23.74.

### Exemplary Preparation of the Salt 2b3e

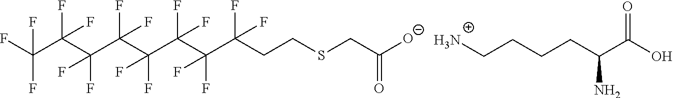 [SMILES: O=C([O-])CSCCC(F)(F)C(F)(F)C(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2b3e, 3,3,4,4,5,5,6,6,7,7,8,8,8-tridecafluoro-1-octanethioacetic acid 2b 0.438 g (1.00 mmol) was dissolved in 20 mL of methanol and a solution of 0.146 g (1.00 mmol) L-lysine in water (0.5 mL) was added. To the reaction mixture, 5 mL water were added and the mixture was refluxed for 2 hours, after which the mixture was cooled and concentrated to dryness to give the product 0.575 g (yield=98%).

### Spectral Analysis:

¹H NMR (500 MHz, D₂O/CD₃CN): δ=3.71 (t, J=6.1 Hz, 1H), 3.27 (s, 2H), 2.99 (t, J=7.5 Hz, 2H), 2.84-2.75 (m, 2H), 2.47 (td, J=18.7, 9.5 Hz, 2H), 1.94-1.82 (m, 2H), 1.74-1.64 (m, 2H), 1.55-1.36 (m, 2H), 1.18 (s, 2H);

¹⁹F NMR (470 MHz, D₂O/CD₃CN): δ=−81.47-−83.36 (m, 3F), −113.76-−116.07 (m, 2F), −122.73 (s, 2F), −123.78 (s, 2F), −124.04 (s, 2F), −127.26 (d, J=15.6 Hz, 2F);

¹³C NMR (126 MHz, D₂O/CD₃CN): δ=176.51, 174.37, 54.53, 39.09, 36.91, 30.98, 29.92, 26.42, 22.67, 21.49.

### Exemplary Preparation of the Salt 2b3f

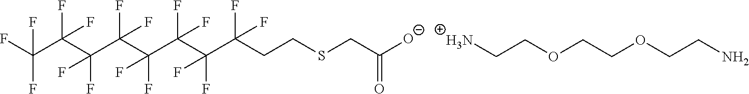 [SMILES: O=C([O-])CSCCC(F)(F)C(F)(F)C(F)(F)C(F)(F)C(F)(F)C(F)(F)C(F)(F)C(F)(F)F ; NCCOCCOCC[NH3+]]

In an exemplary method of preparation of the exemplary salt 2b3f, 3,3,4,4,5,5,6,6,7,7,8,8,8-tridecafluoro-1-octanethioacetic acid 2b 0.438 g (1.00 mmol) was dissolved in 20 mL of methanol and 0.148 g (1.00 mmol) of 2.2′-(ethylenedioxy)-bis(ethylamine) was added. After refluxing for2 hours, the mixture was cooled and concentrated to dryness to give the product 0.580 g (yield=99%).

### Spectral Analysis:

¹H NMR (500 MHz, D₂O/CD₃CN): δ=3.70 (s, 4H), 3.69-3.66 (m, 4H), 3.28 (s, 2H), 3.06-3.00 (m, 4H), 2.84-2.77 (m, 2H), 2.46 (ddd, J=26.5, 18.3, 7.9 Hz, 2H), 2.05 (dt, J=5.0, 2.5 Hz, 1H);

¹⁹F NMR (470 MHz, D₂O/CD₃CN): δ=−81.43-−83.90 (m, 3F), −114.48-−115.28 (m, 2F), −122.83 (s, 2F), −123.75 (d, J=130.9 Hz, 2F), −124.09 (s, 2F), −127.47 (dd, J=27.4, 12.4 Hz, 2F);

¹³C NMR (126 MHz, D₂O/CD₃CN): δ=176.54, 69.59, 68.78, 39.45, 37.34, 31.01, 22.68.

### Exemplary Preparation of the Salt 2b3g

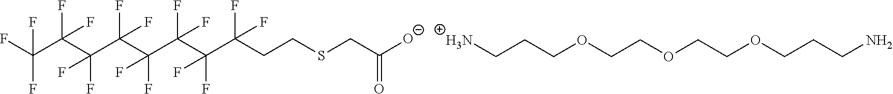 [SMILES: O=C([O-])CSCCC(F)(F)C(F)(F)C(F)(F)C(F)(F)C(F)(F)C(F)(F)C(F)(F)C(F)(F)F ; NCCCOCCOCCOCCC[NH3+]]

In an exemplary method of preparation of the exemplary salt 2b3g, 3,3,4,4,5,5,6,6,7,7,8,8,8-tridecafluoro-1-octanethioacetic acid 2b 0.438 g (1.00 mmol) was dissolved in 20 mL of methanol and 0.220 g (1.00 mmol) of 4,7,10-trioxo-1,13-tridecanediamine was added. After refluxing for 2 hours, the mixture was cooled and concentrated to dryness to give the product 0.650 g (yield=99%).

### Spectral Analysis:

¹H NMR (500 MHz, D₂O/CD₃CN): δ=3.57 (s, 4H), 3.54 (t, J=6.1 Hz, 4H), 3.21 (s, 2H), 2.94-2.88 (m, 4H), 2.77-2.68 (m, 2H), 2.36 (s, 2H), 1.87-1.77 (m, 4H);

¹⁹F NMR (470 MHz, D₂O/CD₃CN): δ=−81.43-−84.90 (m, 2F), −115.13 (s, 2F), −120.96-−123.64 (m, 4F), −124.18 (s, 2F), −127.76 (s, 2F);

¹³C NMR (126 MHz, D₂O/CD₃CN): δ=176.36, 69.52, 69.32, 68.51, 37.68, 37.36, 29.66, 28.03, 22.62.

### Exemplary Preparation of the Salt 2b3l

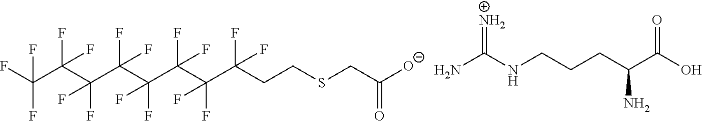 [SMILES: O=C([O-])CSCCC(F)(F)C(F)(F)C(F)(F)C(F)(F)C(F)(F)C(F)(F)C(F)(F)C(F)(F)F ; NC(=[NH2+])NCCC[C@H](N)C(=O)O]

In an exemplary method of preparation of the exemplary salt 2b3l, 3,3,4,4,5,5,6,6,7,7,8,8,8-tridecafluoro-1-octanethioacetic acid 2b 0.438 g (1.00 mmol) was dissolved in 20 mL of methanol and a solution of 0.174 g (1.00 mmol) L-arginine in water (0.1 mL) was added. After refluxing for 2 hours the mixture was cooled and concentrated to dryness to give the product 0.600 g (yield=98%).

### Spectral Analysis:

¹H NMR (500 MHz, D₂O/CD₃OD): δ=3.72-3.64 (m, 1H), 3.26-3.19 (m, 4H), 2.83-2.76 (m, 2H), 2.48 (ddd, J=26.4, 18.3, 8.0 Hz, 2H), 1.98-1.82 (m, 3H), 1.80-1.60 (m, 3H);

¹⁹F NMR (470 MHz, D₂O/CD₃OD): δ=−82.16 (t, J=10.0 Hz, 3F), −114.67-−115.50 (m, 2F), −122.84 (s, 2F), −123.81 (s, 2F), −124.24 (s, 2F), −127.19 (dd, J=14.3, 8.2 Hz, 2F);

¹³C NMR (126 MHz, D₂O/CD₃OD): δ=176.69, 173.76, 156.92, 54.28, 40.54, 36.91, 31.07, 27.78, 24.13, 22.63.

### Exemplary Preparation of the Salt 2c3a

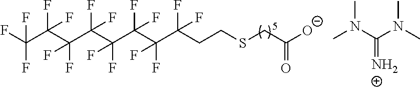

In an exemplary method of preparation of the exemplary salt 2c3a, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and 0.115 g (1.00 mmol) of TMG was added. After refluxing for 2 hours, the mixture was cooled and concentrated to dryness to give the product 0.708 g (yield=100%).

### Spectral Analysis:

¹H NMR (500 MHz, CD₃OD): δ=4.85 (s, 2H), 2.98 (s, 12H), 2.74 (dd, J=9.3, 6.8 Hz, 2H), 2.59 (t, J=7.4 Hz, 2H), 2.45 (ddd, J=26.8, 18.2, 8.3 Hz, 2H), 2.19 (dd, J=16.4, 9.0 Hz, 2H), 1.69-1.55 (m, 4H), 1.50-1.39 (m, 2H);

¹⁹F NMR (470 MHz, CD₃OD): δ=−82.33-−82.46 (m, 3F), −115.38 (dd, J=31.1, 15.8 Hz, 2F), −122.78 (d, J=56.7 Hz, 2F), −122.91 (s, 4F), −123.75 (s, 2F), −124.30 (d, J=73.4 Hz, 2F), −126.98-−127.90 (m, 2F);

¹³C NMR (126 MHz, CD₃OD): δ=180.03, 161.86, 38.46, 36.75, 31.69, 31.41, 28.92, 28.34, 25.52, 21.86.

### Exemplary Preparation of the Salt 2c3c

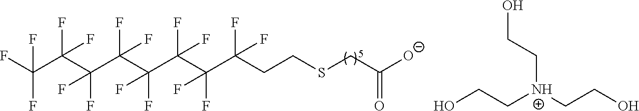

In an exemplary method of preparation of the exemplary salt 2c3c, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and 0.149 g (1.00 mmol) of triethanolamine was added. After refluxing for 2 hours the mixture was cooled and concentrated to dryness to give the product 0.680 g (yield=98%).

### Spectral Analysis:

¹H NMR (500 MHz, D₂O/acetone-d₆): δ=3.97 (t, J=15.2, 9.8 Hz, 6H), 3.34 (t, J=5.3 Hz, 5H), 2.89-2.76 (m, 2H), 2.75-2.63 (m, 2H), 2.55-2.37 (m, 2H), 2.39-2.28 (m, 2H), 2.30-2.24 (m, 2H), 1.77-1.60 (m, 4H), 1.51 (d, J=4.9 Hz, 2H);

¹⁹F NMR (470 MHz, D₂O/acetone-d₆): δ=−81.57-−84.52 (m, 4F), −115.42 (s, 2F), −122.83 (s, 2F), −123.09 (s, 4F), −123.24 (s, 2F), −124.24 (s, 2F), −127.88 (s, 2F);

¹³C NMR (126 MHz, D₂O/acetone-d₆): δ=176.94, 56.41, 55.57, 36.15, 31.85, 29.74, 28.41, 25.24, 22.12, 20.12.

### Exemplary Preparation of the Salt 2c3e

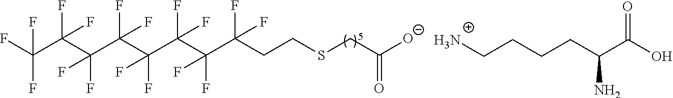

In an exemplary method of preparation of the exemplary salt 2c3e, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and a solution of 0.146 g (1.00 mmol) L-lysine in water (0.5 mL) was added. To the reaction mixture, 5 mL water was added and the mixture was heated to reflux for 2 hours, after which the mixture was cooled and concentrated to dryness to give the product 0.740 g (yield=100%).

### Spectral Analysis:

¹H NMR (500 MHz, D₂O): δ=3.65 (s, 1H), 2.99 (t, J=7.4 Hz, 2H), 2.75-2.63 (m, 2H), 2.57 (t, J=7.2 Hz, 2H), 2.43-2.24 (m, 2H), 2.18 (t, J=7.2 Hz, 2H), 1.95-1,79 (m, 2H), 1.71 (dt, J=14.9, 7.5 Hz, 2H), 1.65-1.53 (m, 4H), 1.53-1.29 (m, 4H);

¹⁹F NMR (470 MHz, D₂O): δ=−81.95-−84.81 (m, 3F), −114.61 (s, 2F), −115.81 (s, 2F), −123.06 (s, 2F), −123.37 (d, J=66.7 Hz, 2F), −124.32 (s, 2F), −124.56 (s, 2F), −128.05 (s, 2F);

¹³C NMR (126 MHz, D₂O): δ=181.68, 174.57, 54.38, 38.88, 37.24, 31.59, 30.20, 28.72, 28.34, 26.47, 25.57, 21.94, 21.48.

### Exemplary Preparation of the Salt 2c3l

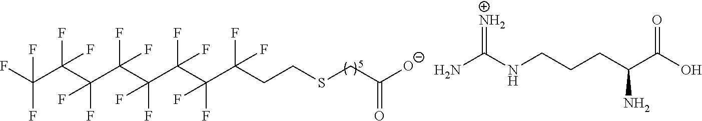

In an exemplary method of preparation of the exemplary salt 2c3l, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and a solution of 0.174 g (1.00 mmol) L-arginine in water (1 mL) was added. After refluxing for 2 hours the mixture was cooled and concentrated to dryness to give the product 0.750 g (yield=98%).

### Spectral Analysis:

¹H NMR (500 MHz, D₂O/CD₃CN): δ=3.82-3.67 (m, 1H), 3.65-3.55 (m, 2H), 3.20 (t, J=6.6 Hz, 2H), 2.81-2.65 (m, 2H), 2.58 (t, J=7.2 Hz, 2H), 2.34 (s, 2H), 2.23-2.10 (m, 2H), 2.04 (dt, J=4.9, 2.5 Hz, 2H), 1.98-1.76 (m, 4H), 1.75-1.48 (m, 4H), 1.46-1.30 (m, 2H);

¹⁹F NMR (470 MHz, D₂O/CD₃CN): δ=−83.21 (s, 3F), −113.12-−114.68 (m, 2F), −115.40 (s, 2F), −122.83 (s, 2F), −123.07 (s, 2F), −123.20 (s, 2F), −124.16 (s, 2F), −127.89 (s, 2F);

¹³C NMR (126 MHz, D₂O/CD₃CN): δ=181.81, 174.36, 155.71, 56.27, 40.71, 32.24, 31.13, 30.63, 30.46, 28.83, 28.12, 25.69, 25.64, 24.21.

### Exemplary Preparation of the Salt 2c3f

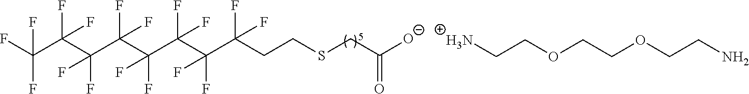

In an exemplary method of preparation of the exemplary salt 2c3f, 3,3,4,4,5,5,6,6,7,7,8,8,9,9,9,10,10,10-heptadecafluoro-1-decane-6-thiohexanoic acid 2c 0.594 g (1.00 mmol) in 20 mL of methanol was dissolved and 0.148 g (1.00 mmol) of 2.2′-(ethylenedioxy)-bis(ethylamine) was added. After refluxing for 2 hours the mixture was cooled and concentrated to dryness to give the product 0.736 g (yield=98%).

### Spectral Analysis:

¹H NMR (500 MHz, CD₃OD): δ=3.67 (s, 4H), 3.62 (dd, J=11.7, 6.4 Hz, 4H), 2.96 (dd, J=11, 4, 6.1 Hz, 4H), 2.74 (dd, J=9.3, 6.8 Hz, 2H), 2.63-2.56 (m, 2H), 2.45 (ddd, J=26.0, 18.1, 8.0 Hz, 2H), 2.16 (t, J=7.5 Hz, 2H), 1.66-1.57 (m, 4H), 1.49-1.39 (m, 2H), 1.28 (s, 1H);

¹⁹F NMR (470 MHz, CD₃OD): δ=−82.33-−82.64 (m, 3F), −115.17-−115.56 (m, 2F), −122.72 (s2F), −122.90 (s, 4F), −123.75 (s, 2F), −124.37 (s, 2F), −127.13-−127.48 (m, 2F);

¹³C NMR (126 MHz, CD₃OD): δ=181.26, 69.89, 69.30, 39.87, 37.56, 31.69, 31.43, 28.96, 28.45, 25.84 , 21.86.

### Exemplary Preparation of the Salt 2c3g

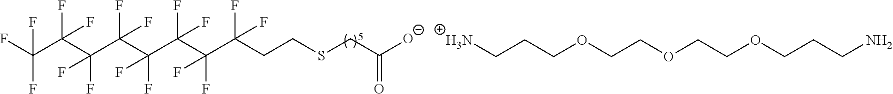

In an exemplary method of preparation of the exemplary salt 2c3g, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and 0.220 g (1.00 mmol) of 4,7,10-trioxo-1,13-tridecanediamine was added. After refluxing for 2 hours the mixture was cooled and concentrated to dryness to give the product 0.810 g (yield=98%).

### Spectral Analysis:

¹H NMR (500 MHz, CD₃OD): δ=3.66-3.62 (m, 4H), 3.59 (h, J=4.2 Hz, 4H), 2.89 (dt, J=10.7, 6.0 Hz, 4H), 2.74 (dd, J=9.3, 6.8 Hz, 2H), 2.62-2.55 (m, 2H), 2.52-2.37 (m, 2H), 2.16 (dd, J=14.7, 7.3 Hz, 2H), 1.87-1.77 (m, 4H), 1.69-1.57 (m, 4H), 1.49-1.39 (m, 2H);

¹⁹F NMR (470 MHz, CD₃OD): δ=−82.31-−82.51 (m, 3F), −115.37 (dd, J=30.9, 15.7 Hz, 2F), −122, 77 (d, J=52.9 Hz, 2F), −122.91 (s, J=90.2 Hz, 4F), −123.86 (d, J=104.1 Hz, 2F), −124.37 (s, 2F), −127.12-−127.48 (m, 2F);

¹³C NMR (126 MHz, CD₃OD): δ=181.26, 69.90, 69.66, 68.83, 38.40, 37.65, 31.44, 29.19, 28.97, 28.47, 25.88, 21.86.

### Exemplary Preparation of the Salt 2c3k

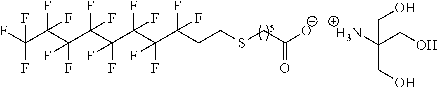

In an exemplary method of preparation of the exemplary salt 2c3k, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and a solution of 0.121 g (1.00 mmol) trizma-base (tri(hydroxymethyl)aminomethane) in water (0.5 mL) was added. After refluxing for 2 hours the mixture was cooled and concentrated to dryness to give the product 0.710 g (yield=99%).

### Spectral Analysis:

¹H NMR (500 MHz, D₂O/CD₃CN): δ=3.97 (s, 9H), 3.10-3.04 (m, 2H), 2.93 (dd, J=17.7, 10.4 Hz, 2H), 2.80-2.63 (m, 2H), 2.52 (t, J=7.5 Hz, 2H), 1.98-1.88 (m, 4H), 1.74 (dd, J=14.8, 7.9 Hz, 2H);

¹⁹F NMR (470 MHz, D₂O/CD₃CN): δ=−82.71 (dt, J=50.0, 10.2 Hz, 3F), −115.09 (dd, J=73.3, 58.4 Hz, 2F), −122.54 (s, 2F), −122.82 (d, J=50.8 Hz, 4F), −123.77 (s, 2F), −123.95 (s, 2F), −127.43 (s, 2F);

¹³C NMR (126 MHz, D₂O/CD₃CN): δ=181.61, 60.95, 60.01, 39.24, 37.27, 31.98, 29.15, 28.73, 25.80, 22.29.

### Exemplary Preparation of the Salt 2cNa

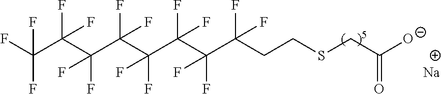

In an exemplary method of preparation of the exemplary salt 2cNa, 3,3,4,4,5,5,6,6,7,7,8,8,9,9,9,10,10,10-heptadecafluoro-1-decane-6-thiohexanoic acid 2c 0.594 g (1.00 mmol) was dissolved in 20 mL of methanol and a solution of 0.040 g (1.00 mmol) sodium hydroxide in water/methanol (ratio 0.1 mL/1 mL, respectively) was added. After refluxing for 2 hours the mixture was cooled and concentrated to dryness to give the product 0.610 g (yield=98%).

### Spectral Analysis:

¹H NMR (500 MHz, D₂O): δ=2.89-2.79 (m, 2H), 2.70 (t, J=7.2 Hz, 2H), 2.55-2.40 (m, 2H), 2.28 (dd, J=15.0, 7.5 Hz, 2H), 1.77-1.62 (m, 4H), 1.55-1.41 (m, 2H);

¹⁹F NMR (470 MHz, D₂O): δ=−83.18 (s, 3F), −114.81 (d, J=559.6 Hz, 2F), −122.84 (s, 2F), −123.16 (d, J=74.2 Hz, 4F), −124.19 (d, J=57.4 Hz, 2F), −127.88 (s, 2F);

¹³C NMR (126 MHz, D₂O): δ=182.74, 37.64, 31.81, 28.88, 28.53, 25.76, 25.08, 22.11.

### Exemplary Preparation of the Salt 2d3c

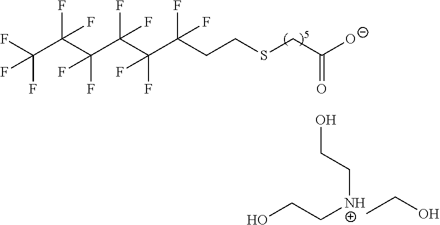

In an exemplary method of preparation of the exemplary salt 2d3c, 3,3,4,4,5,5,6,6,7,7,8,8,8-tridecafluoro-1-octa-6-thiohexanoic acid 2d 0.494 g (1.00 mmol) was dissolved in 20 mL of methanol and 0.149 g (1.00 mmol) of triethanolamine was added. After refluxing for 2 hours the mixture was cooled and concentrated to dryness to give the product 0.635 g (yield=99%).

### Spectral Analysis:

¹H NMR (500 MHz, D₂O/CD₃CN): δ=3.87 (t, J=5.5 Hz, 6H), 3.24 (t, J=5.5 Hz, 6H), 2.73 (dd, J=18.8, 10.5 Hz, 2H), 2.65-2.55 (m, 2H), 2.39 (dq, J=17.6, 10.3 Hz, 2H), 2.22 (t, J=7.6 Hz, 2H), 1.61 (tt, J=15.3, 7.6 Hz, 4H), 1.42 (dt, J=15.0, 7.6 Hz, 2H);

¹⁹F NMR (470 MHz, D₂O/CD₃CN): δ=−81.12-−84.89 (m, 3F), −114.60-−115.67 (m, 2F), −122.94 (s, 2F), −123.99 (s, 2F), −124.25 (s, 2F), −127.55 (d, J=15.5 Hz, 2F);

¹³C NMR (126 MHz, D₂O/CD₃CN): δ=180.98, 56.37, 55.54, 36.57, 31.75, 31.56, 28.89, 28.43, 25.40, 22.13.

### Exemplary Preparation of the Salt 2d3e

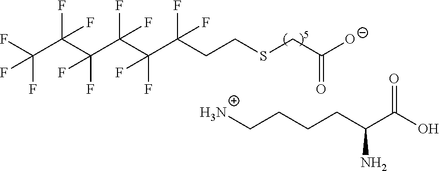

In an exemplary method of preparation of the exemplary salt 2d3e, 3,3,4,4,5,5,6,6,7,7,8,8,8-tridecafluoro-1-octa-6-thiohexanoic acid 2d 0.494 g (1.00 mmol) was dissolved in 20 mL of methanol and a solution of 0.146 g (1.00 mmol) L-lysine in water (0.5 mL) was added. Then 5 mL of water was added and the mixture was refluxed for 2 hours, after which was cooled and concentrated to dryness to obtain the product 0.690 g (yield =99%).

### Spectral Analysis:

¹H NMR (500 MHz, D₂O/CD₃CN): δ=3.77 (d, J=5.9 Hz, 2H), 3.08 (t, J=7.4 Hz, 2H), 2.87-2.77 (m, 2H), 2.67 (t, J=7.1 Hz, 2H), 2.46 (d, J=8.2 Hz, 2H), 2.26 (t, J=7, 4 Hz, 2H), 2.13 (s, 1H), 1.95 (s, 2H), 1.86-1.74 (m, 4H), 1.75-1.61 (m, 4H), 1.59-1.37 (m, 6H);

¹⁹F NMR (470 MHz, D₂O/CD₃CN): δ=−83.04 (d, J=154.1 Hz, 3F), −115.31 (s, 2F), −122.99 (s, 2F), −124.04 (s, 2F), −124.31 (s, 2F), −127.63 (s, 2F);

¹³C NMR (126 MHz, D₂O/CD₃CN): δ=182.15, 175.26, 54.57, 39.05, 37.18, 31.66, 31.45, 30.25, 28.76, 28.37, 26.45, 25.54, 22.06, 21.50.

### Exemplary Preparation of the Salt 2d3l

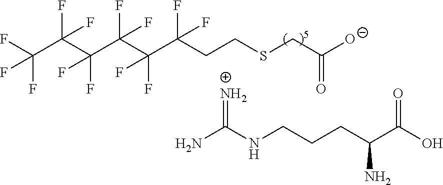

In an exemplary method of preparation of the exemplary salt 2d3l, 3,3,4,4,5,5,6,6,7,7,8,8,8-tridecafluoro-1-octa-6-thiohexanoic acid 2d 0.494 g (1.00 mmol) was dissolved in 20 mL of methanol and a solution of 0.174 g (1.00 mmol) L-arginine in water (0.5 mL) was added. Then 5 mL of water was added and the mixture was refluxed for 2 hours, followed by cooling and concentrating to dryness to obtain the product 0.690 g (yield 99%).

### Spectral Analysis:

¹H NMR (500 MHz, D₂O/CD₃CN): δ=3.63 (t, J=6.2 Hz, 1H), 3.22 (t, J=7.0 Hz, 2H), 2.75-2.67 (m, 2H), 2.56 (dd, J=16.8, 9.5 Hz, 2H), 2.36 (ddd, J=36.0, 22.3, 7.3 Hz, 2H), 2.16 (t, J=7.5 Hz, 2H), 1.94-1.82 (m, 2H), 1.76-1.50 (m, 6H), 1.39 (dt, J=14.7, 7.3 Hz, 2H);

¹⁹F NMR (470 MHz, D₂O/CD₃CN): δ=−81.98-−83.09 (m, 3F), −115.32 (s, 2F), −122.96 (s, 2F), −123.96 (s, 2F), −124.38 (s, 2F), −127.42 (s, 2F);

¹³C NMR (126 MHz, D₂O/CD₃CN): δ=182.23, 174.64, 156.95, 54.39, 40.59, 37.54, 31.56, 28.78, 28.36, 28.18, 25.71, 24.21, 21.96.

### Exemplary Preparation of the Salt 2d3f

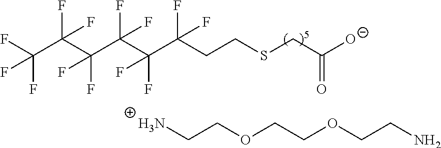

In an exemplary method of preparation of exemplary salt 2d3f, 3,3,4,4,5,5,6,6,7,7,8,8,8-tridecafluoro-1-octa-6-thiohexanoic acid 2d 0.494 g (1.00 mmol) was dissolved in 20 mL of methanol and 0.148 g (1.00 mmol) of 2.2′-(ethylenedioxy)-bis(ethylamine) was added. After refluxing for 2 hours the mixture was cooled and concentrated to dryness to give the product 0.628 g (yield=98%).

### Spectral Analysis:

¹H NMR (500 MHz, D₂O/CD₃CN): δ=3.85-3.75 (m, 8H), 3.18 (dd, J=14.5, 9.4 Hz, 4H), 2.80 (s, 2H), 2.67 (d, J=6.5 Hz, 2H), 2.44 (s, 2H), 2.25 (s, 2H), 1.68 (d, J=22.7 Hz, 4H), 1.49 (s, 2H);

¹⁹F NMR (470 MHz, D₂O/CD₃CN): δ=−81.15-−85.38 (m, 3F), −115.40 (s, 2F), −122.68 (d, J=346.8 Hz, 2F), −124.32 (s, 2F), −127.83 (s, 2F);

¹³C NMR (126 MHz, D₂O/CD₃CN): δ=181.11, 69.60, 67.91, 39.22, 37.64, 31.87, 31.46, 28.96, 28.60, 25.82, 22.18.

### Exemplary Preparation of the Salt 2d3g

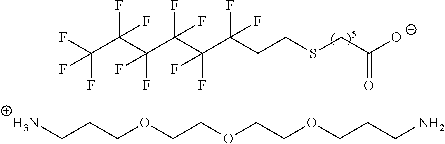

In an exemplary method of preparation of the exemplary salt 2d3g, 3,3,4,4,5,5,6,6,7,7,8,8,8-tridecafluoro-1-octa-6-thiohexanoic acid 2d 0.494 g (1.00 mmol) was dissolved in 20 mL of methanol and 0.220 g (1.00 mmol) of 4,7,10-trioxo-1,13-tridecanediamine was added. After refluxing for 2 hours the mixture was cooled and concentrated to dryness to give the product 0.710 g (99% yield).

### Spectral Analysis:

¹H NMR (500 MHz, D₂O/CD₃CN): δ=3.92-3.80 (m, 12H), 3.21 (dd, J=13.1, 6.0 Hz, 4H), 2.96-2.89 (m, 2H), 2.80 (t, J=7.4 Hz, 2H), 2.66-2.50 (m, 2H), 2.38-2.31 (m, 2H), 2.17-2.04 (m, 4H), 1.79 (qd, J=15.2, 7.6 Hz, 4H), 1.59 (dt, J=15.1, 7.5 Hz, 2H);

¹⁹F NMR (470 MHz, D₂O/CD₃CN): δ=−81.78-−83.50 (m, 3F), −115.15 (d, J=15.6 Hz, 2F), −122.90 (s, 2F), −123.96 (s, 2F), −124.19 (s, 2F), −127.52 (d, J=14.1 Hz, 2F);

¹³C NMR (126 MHz, D₂O/CD₃CN): δ=182.27, 69.71, 69.55, 68.62, 37.83, 37.80, 31.88, 29.05, 28.68, 27.75, 25.93, 22.18.

### Exemplary Preparation of the Salt 2i3c

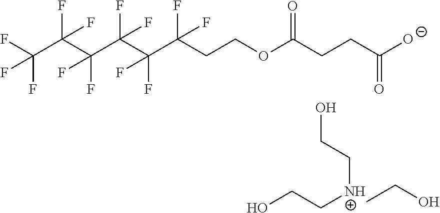

In an exemplary method of preparation of the exemplary salt 2i3c, 1H,1H,2H,2H-perfluoro-1-octyl succinic acid monoester 2i 1.8 g (3.88 mmol) was dissolved in 5 mL of methanol and 0.58 g (3.88 mmol) of triethanolamine was added. After heating to complete dissolution, the mixture was cooled and concentrated to dryness to give the product 2.33 g (yield=98%).

### Spectral Analysis:

¹H NMR (500 MHz, CDCl₃): δ=6.24 (s, 3H), 4.36 (t, J=6.7 Hz, 2H), 3.87-3.74 (m, 6H), 3.08-3.00 (m, 6H), 2.61-2.39 (m, 6H);

¹⁹F NMR (470 MHz, CDCl₃): δ=−80.91 (t, J=10.0 Hz, 3F), −112.84-−114.39 (m, 2F), −121.69-−122.15 (m, 2F), −122.75-−123.16 (m, 2F), −123.50-−124.09 (m, 2F), −126.02-−126.56 (m, 2F);

¹³C NMR (126 MHz, CDCl₃): δ=177.88, 172.95, 57.55, 57.43, 56.32, 30.62, 30.40, 29.76.

### Exemplary Preparation of the Salt 2i3e

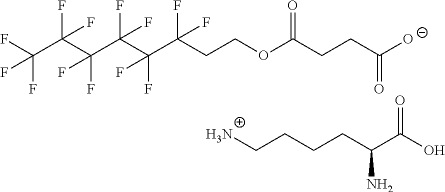 [SMILES: O=C([O-])CCC(=O)OCCC(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2i3e, 1H,1H,2H,2H-perfluoro-1-octyl succinic acid monoester 2i 0.600 g (1.29 mmol) was dissolved in 2 mL of methanol and a solution of 0.188 g (1.29 mmol) L-lysine in water (0.5 mL) was added. After heating to complete dissolution, the mixture was cooled and concentrated to dryness to give the product 0.756 g (yield=96%).

### Spectral Analysis:

¹H NMR (500 MHz, D₂O): δ=4.23 (t, J=6.3 Hz, 2H), 3.62 (t, J=6.1 Hz, 1H), 2.96-2.83 (m, 2H), 2.49-2.25 (m, 6H), 1.87-1.70 (m, 2H), 1.59 (dt, J=15.0, 7.7 Hz, 2H), 1.44-1.25 (m, 2H);

¹⁹F NMR (470 MHz, D₂O): δ=−82.93 (s, 3F), −114.80 (s, 2F), −123.01 (s, 2F), −124.10 (s, 2F), −124.73 (s, 2F), −127.68 (s, 2F);

¹³C NMR (126 MHz, D₂O): δ=179.79, 174.75, 174.63, 56.62, 54.40, 38.95, 31.28, 29.88, 29.80, 29.60, 26.33, 21.38.

### Exemplary Preparation of the Salt 2iK

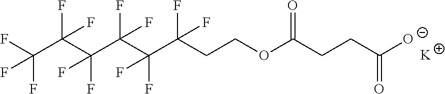 [SMILES: O=C([O-])CCC(=O)OCCC(F)(F)C(F)(F)C(F)(F)C(F)(F)C(F)(F)C(F)(F)F ; [K+]]

In an exemplary method of preparation of the exemplary salt 2iK, 1H,1H,2H,2H-perfluoro-1-octyl succinic acid monoester 2i 0.500 g (1.08 mmol) was dissolved in 2 mL of methanol and a solution of 0.149 g (1.08 mmol) K₂CO₃ in water (1 mL) was added. After heating to complete dissolution, the mixture was cooled and concentrated to dryness to give the product 0.524 g (yield=97%).

### Spectral Analysis:

¹H NMR (500 MHz, CD₃OD): δ=4.41-4.35 (m, 1H), 2.60-2.54 (m, 2H), 2.51-2.44 (m, 2H);

¹⁹F NMR (470 MHz, CD₃OD): δ=−82.42-−82.52 (m, 3F), −114.58-−114.80 (m, 2F), −122.94 (s, 2F), −123.94 (s, 2F), −124.67 (s, 2F), −127.14-−127.72 (m, 2F);

¹³C NMR (126 MHz, CD₃OD): δ=178.20, 173.24, 55.93, 33.40, 31.35, 30.00.

Exemplary Preparation of the Salt 2i2i3i

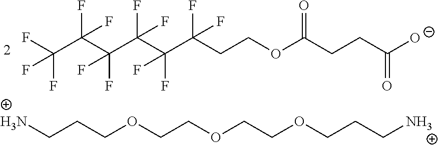 [SMILES: O=C([O-])CCC(=O)OCCC(F)(F)C(F)(F)C(F)(F)C(F)(F)C(F)(F)C(F)(F)F ; [NH3+]CCCOCCOCCOCCC[NH3+]]

In an exemplary method of preparation of the exemplary salt 2i2i3i, 1H,1H,2H,2H-perfluoro-1-octyl succinic acid monoester 2i 0.500 g (1.08 mmol) was dissolved in 2 mL of methanol and 0.118 g (0.538 mmol) of 4,7,10-trioxo-1,13-tridecanediamine was added. After heating to complete dissolution, the mixture was cooled and concentrated to dryness to give the product 0.610 g (99% yield).

### Spectral Analysis:

¹H NMR (500 MHz, CDCl₃): δ=5.70 (s, 3H), 4.36 (t, J=6.5 Hz, 2H), 3.62 (d, J=21.5 Hz, 6H), 3.08-3.00 (m, J=5.8 Hz, 2H), 2.59-2.51 (m, 2H), 2.51-2.34 (m, 4H), 1.97-1.87 (m, 2H);

¹⁹F NMR (470 MHz, CDCl₃): δ=−80.64-−81.16 (m, 3F), −113.68-−114.00 (m, 2F), −122.02 (s, 2F), −122.99 (s, 2F), −123.70 (s, 2F), −126.13-−126.34 (m, 2F);

¹³C NMR (126 MHz, CDCl₃): δ=178.47, 173.53, 70.06, 69.74, 69.21, 56.08, 38.19, 32.11, 30.59, 30.41, 27.01.

Exemplary Preparation of the Salt 2i2i3h

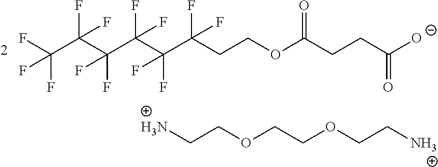 [SMILES: O=C([O-])CCC(=O)OCCC(F)(F)C(F)(F)C(F)(F)C(F)(F)C(F)(F)C(F)(F)F ; [NH3+]CCOCCOCC[NH3+]]

In an exemplary method of preparation of the exemplary salt 2i2i3h, 1H,1H,2H,2H-perfluoro-1-octyl succinic acid monoester 2i 0.500 g (1.08 mmol) was dissolved in 2 mL of methanol and 0.080 g (0.538 mmol) of 2.2′-(ethylenedioxy)-bis(ethylamine) was added. After heating to complete dissolution, the mixture was cooled and concentrated to dryness to give the product 0.568 g (yield=98%).

### Spectral Analysis:

¹H NMR (500 MHz, CDCl₃): δ=5.91 (s, 3H), 4.36 (t, J=6.4 Hz, 2H), 3.73-3.55 (m, 4H), 3.07 (s, 2H), 2.56 (t, J=6.6 Hz, 2H), 2.50-2.41 (m, 4H);

¹⁹F NMR (470 MHz, CDCl₃): δ=−80.67-−81.44 (m, 3F), −113.62-−114.18 (m, 2F), −122.04 (s, 2F), −123.02 (s, 2F), −123.74 (s, 2F), −126.07-−126.56 (m, 2F);

¹³C NMR (126 MHz, CDCl₃): δ=178.90, 173.52, 69.33, 66.62, 56.12, 39.15, 33.84, 32.03, 30.41.

### Exemplary Preparation of the Salt 2i3k

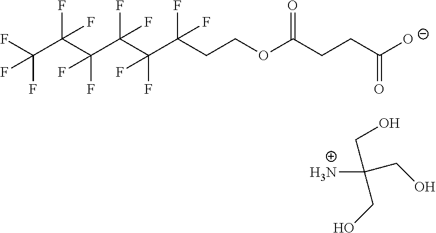 [SMILES: O=C([O-])CCC(=O)OCCC(F)(F)C(F)(F)C(F)(F)C(F)(F)C(F)(F)C(F)(F)F ; [NH3+]C(CO)(CO)CO]

In an exemplary method of preparation of the exemplary salt 2i3k, 1H,1H,2H,2H-perfluoro-1-octyl succinic acid monoester 2i 0.400 g (0.86 mmol) was dissolved in 2 mL of methanol and 0.104 g (0.86 mmol) of trizma-base (tri(hydroxymethyl)aminomethane) was added. After heating to complete dissolution, the mixture was cooled and concentrated to dryness to give the product 0.492 g (yield=98%).

### Spectral Analysis:

¹H NMR (500 MHz, CD₃OD): δ=4.38 (t, J=6.4 Hz, 2H), 3.65 (s, 6H), 2.63-2.53 (m, 4H), 2.52-2.44 (m, 2H);

¹⁹F NMR (470 MHz, CD₃OD): δ=−82.45 (t, J=10.2 Hz, 3F), −114.60-−114.83 (m, 2F), −122.93 (s, 2F), −123.93 (s, 2F), −124.66 (s, 2F), −127.19-−127.60 (m, 2F);

¹³C NMR (126 MHz, CD₃OD): δ=179.99, 174.61, 62.15, 61.49, 57.33, 32.98, 31.49, 31.30.

### Exemplary Preparation of the Salt 2i3l

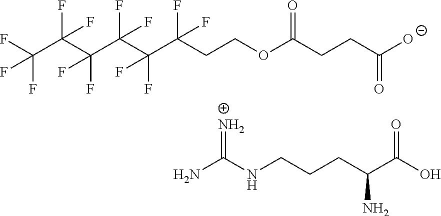 [SMILES: O=C([O-])CCC(=O)OCCC(F)(F)C(F)(F)C(F)(F)C(F)(F)C(F)(F)C(F)(F)F ; NC(=[NH2+])NCCC[C@H](N)C(=O)O]

In an exemplary method of preparation of the exemplary salt 2i3l, 1H,1H,2H,2H-perfluoro-1-octyl succinic acid monoester 2i 0.350 g (0.75 mmol) was dissolved in 2 mL of methanol and a solution of 0.131 g (0.75 mmol) L-arginine in water (0.5 mL) was added. After heating to complete dissolution, the mixture was cooled and concentrated to dryness to give the product 0.476 g (yield=99%).

### Spectral Analysis:

¹H NMR (500 MHz, D₂O): δ=4.23 (t, J=6.3 Hz, 2H), 3.61 (t, J=6.1 Hz, 1H), 3.10 (t, J=6.9 Hz, 2H), 2.44-2.28 (m, 6H), 1.83-1.71 (m, 2H), 1.64-1.47 (m, 2H);

¹⁹F NMR (470 MHz, D₂O): δ=−82.78 (s, 3F), −114.74 (s, 2F), −122.94 (s, 2F), −124.03 (s, 2F), −124.65 (s, 2F), −127.56 (s, 2F);

¹³C NMR (126 MHz, D₂O): δ=182.68, 177.45, 177.33, 159.43, 59.17, 56.92, 43.07, 34.11, 32.56, 32.28, 30.35, 26.58.

### Exemplary Preparation of the Salt 2j3a

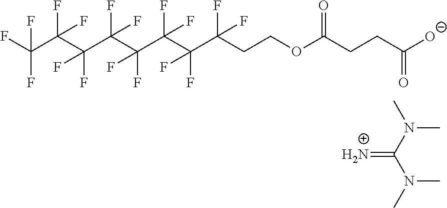

In an exemplary method of preparation of the exemplary salt 2j3a, 1H,1H,2H,2H-perfluoro-1-decyl succinic acid monoester 2j 0.500 g (0.89 mmol) was dissolved in 2 mL of methanol and 0.102 g (0.89 mmol) of TMG was added. After heating to complete dissolution, the mixture was cooled and concentrated to dryness to give the product 0.598 g (yield=99%).

### Spectral Analysis:

¹H NMR (500 MHz, CDCl₃): δ=3.93 (t, J=6.7 Hz, 2H), 2.96 (s, 12H), 2.58 (t, J=7.1 Hz, 2H), 2.47 (t, J=7.0 Hz, 2H), 2.37 (ddd, J=19.1, 12.9, 6.7 Hz, 2H);

¹⁹F NMR (470 MHz, CDCl₃): δ=−80.89 (s, 3F), −113.54 (s, 2F), −121.80 (s, 2F), −122.02 (s, 4F), −122.82 (s, 2F), −123.77 (s, 2F), −126.21 (s, 2F);

¹³C NMR (126 MHz, CDCl₃): δ=178.08, 174.64, 162.66, 54.33, 51.26, 39.56, 33.92, 32.78, 31.03.

### Exemplary Preparation of the Salt 2j3c

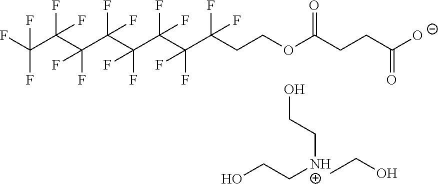

In an exemplary method of preparation of the exemplary salt 2j3c,1H,1H,2H,2H-perfluoro-1-decyl succinic acid monoester 2j 0.500 g (0.89 mmol) was dissolved in 2 mL of methanol and 0.132 g (0.89 mmol) of triethanolamine was added. After heating to complete dissolution, the mixture was cooled and concentrated to dryness to give the product 0.628 g (99% yield).

### Spectral Analysis:

¹H NMR (500 MHz, acetone-d₆): δ=5.15 (s, 3H), 4.43 (t, J=6.3 Hz, 2H), 3.72-3.62 (m, 6H), 2.95-2.86 (m, 6H), 2.68 (tt, J=19.0, 6.2 Hz, 2H), 2.62-2.51 (m, 4H);

¹⁹F NMR (470 MHz, acetone-d₆): δ=−81.71 (t, J=10.1 Hz, 3F), −114.05 (s, 2F), −122.22 (s, 2F), −122.45 (s, 2F), −122.46 (s, 2F), −123.28 (s, 2F), −124.13 (s, 2F), −126.76 (s, 2F);

¹³C NMR (126 MHz, acetone-d₆): δ=174.47, 172.05, 58.63, 57.33, 55.93, 30.03, 29.26, 29.14.

### Exemplary Preparation of the Salt 2j3e

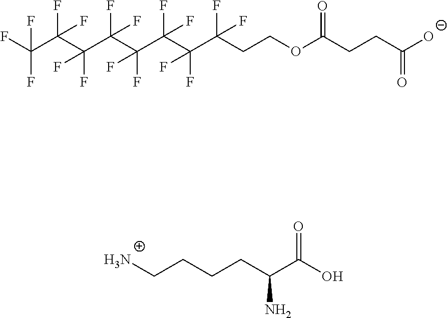 [SMILES: O=C([O-])CCC(=O)OCCC(F)(F)C(F)(F)C(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2j3e, 1H,1H,2H,2H-perfluoro-1-decyl succinic acid monoester 2j 0.500 g (0.89 mmol) was dissolved in 2 mL of methanol and a solution of 0.129 g (0.89 mmol) L-lysine in water (0.5 mL) was added. After heating to complete dissolution, the mixture was cooled and concentrated to dryness to give the product 0.625 g (yield=99%).

### Spectral Analysis:

¹H NMR (500 MHz, D₂O/CD₃CN): δ=4.41 (t, J=6.4 Hz, 2H), 3.77 (t, J=6.1 Hz, 1H), 3.06 (t, J=7.5 Hz, 2H), 2.67-2.50 (m, 6H), 2.00-1.87 (m, 2H), 1.83-1.71 (m, 2H), 1.59-1.46 (m, 2H);

¹⁹F NMR (470 MHz, D₂O/CD₃CN): δ=−83.04 (t, J=10.2 Hz, 3F), −114.60-−114.83 (m, 2F), −122.72 (s, 2F), −122.99 (s, 2F), −123.10 (s, 2F), −124.02 (s, 2F), −124.40 (s, 2F), −127.74 (s, 2F);

¹³C NMR (126 MHz, D₂O/CD₃CN): δ=178.58, 174.29, 174.11, 56.48, 54.58, 39.14, 30.97, 30.01, 29.82, 29.78, 26.50, 21.59.

### Exemplary Preparation of the Salt 2jNa

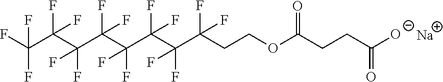 [SMILES: O=C([O-])CCC(=O)OCCC(F)(F)C(F)(F)C(F)(F)C(F)(F)C(F)(F)C(F)(F)C(F)(F)C(F)(F)F ; [Na+]]

In an exemplary method of preparation of the exemplary salt 2jNa, 1H,1H,2H,2H-perfluoro-1-decyl succinic acid monoester 2j 0.100 g (0.18 mmol) was dissolved in 1 mL of methanol and a solution of 0.015 g (0.18 mmol) NaHCO₃ in water (1 mL) was added. After heating to complete dissolution, the mixture was cooled and concentrated to dryness to give the product 0.103 g (yield=99%).

### Spectral Analysis:

¹H NMR (500 MHz, D₂O/CD₃CN): δ=4.44 (t, J=6.5 Hz, 2H), 2.68-2.50 (m, 6H);

¹⁹F NMR (470 MHz, D₂O/CD₃CN): δ=−82.98 (s, 3F), −114.58 (s, 2F), −122.67 (s, 2F), −122.94 (s, 2F), −123.04 (s, 2F), −123.97 (s, 2F), −124.33 (s, 2F), −127.69 (s, 2F);

¹³C NMR (126 MHz, D₂O/CD₃CN): δ=178.96, 174.34, 56.52, 31.21, 29.98, 29.80.

### Exemplary Preparation of the Salt 2jK

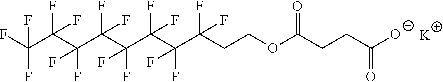 [SMILES: O=C([O-])CCC(=O)OCCC(F)(F)C(F)(F)C(F)(F)C(F)(F)C(F)(F)C(F)(F)C(F)(F)C(F)(F)F ; [K+]]

In an exemplary method of preparation of the exemplary salt 2jK, 1H,1H,2H,2H-perfluoro-1-decyl succinic acid monoester 2j 0.100 g (0.18 mmol) was dissolved in 1 mL of methanol and a solution of 0.012 mg (0.09 mmol) K₂CO₃ in water (1 mL) was added. After heating to complete dissolution, the mixture was cooled and concentrated to dryness to give the product 0.105 g (yield=99%).

### Spectral Analysis:

¹H NMR (500 MHz, D₂O/CD₃CN): δ=4.44-4.28 (m, 2H), 2.63-2.38 (m, 6H);

¹⁹F NMR (470 MHz, D₂O/CD₃CN): δ=−83.13 (s, 3F), −114.70 (s, 2F), −122.77 (s, 2F), −123.06 (s, 4F), −124.08 (s, 2F), −124.44 (s, 2F), −127.82 (s, 2F);

¹³C NMR (126 MHz, D₂O/CD₃CN): δ=178.65, 174.24, 56.46, 48.96, 30.92, 29.73.

Exemplary Preparation of the Salt 2j2j3i

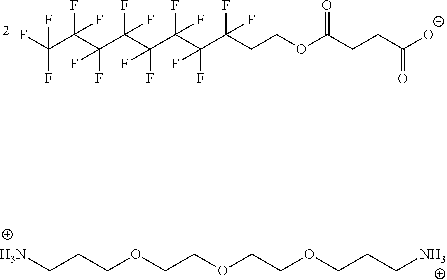 [SMILES: O=C([O-])CCC(=O)OCCC(F)(F)C(F)(F)C(F)(F)C(F)(F)C(F)(F)C(F)(F)C(F)(F)C(F)(F)F ; [NH3+]CCCOCCOCCOCCC[NH3+]]

In an exemplary method of preparation of exemplary salt 2j2j3i, 1H,1H,2H,2H-perfluoro-1-decyl succinic acid monoester 2j 0.400 g (0.71 mmol) was dissolved in 2 mL of methanol and 0.078 g (0.35 mmol) of 4,7,10-trioxo-1,13-tridecanodiamine was added. After heating to complete dissolution, the mixture was cooled and concentrated to dryness to give the product 0.472 g (yield=99%).

### Spectral Analysis:

¹H NMR (500 MHz, CDCl₃): δ=5.49 (s, 3H), 4.36 (t, J=6.7 Hz, 2H), 3.68-3.57 (m, 6H), 3.04 (t, J=6.1 Hz, 2H), 2.56 (t, J=6.9 Hz, 2H), 2.50-2.41 (m, 4H), 1.97-1.91 (m, 2H);

¹⁹F NMR (470 MHz, CDCl₃): δ=−80.82-−80.94 (m, 3F), −113.66-−113.83 (m, 2F), −121.78 (s, 2F), −122.02 (s, 4F), −122.81 (s, 2F), −123.63 (s, 2F), −126.14-−126.42 (m, 2F);

¹³C NMR (126 MHz, CDCl₃): δ=178.39, 173.52, 70.09, 69.73, 69.41, 56.07, 38.34, 32.14, 30.65, 30.44, 27.05.

Exemplary Preparation of the Salt 2j2j3h

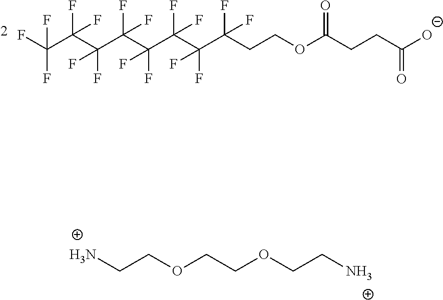 [SMILES: O=C([O-])CCC(=O)OCCC(F)(F)C(F)(F)C(F)(F)C(F)(F)C(F)(F)C(F)(F)C(F)(F)C(F)(F)F ; [NH3+]CCOCCOCC[NH3+]]

In an exemplary method of preparation of the exemplary salt 2j2j3h, 1H,1H,2H,2H-perfluoro-1-decyl succinic acid monoester 2j 0.400 g was dissolved in 2 mL of methanol (0.71 mmol) and 0.052 g (0.35 mmol) of 2.2′-(ethylenedioxy)-bis(ethylamine) was added. After heating to complete dissolution, the mixture was cooled and concentrated to dryness to give the product 0.447 g (yield=99%).

### Spectral Analysis:

¹H NMR (500 MHz, CDCl₃): δ=5.83 (s, 3H), 4.36 (t, J=6.6 Hz, 2H), 3.68-3.61 (m, 4H), 3.08-3.02 (m, 2H), 2.59-2.53 (m, 2H), 2.49-2.41 (m, 4H);

¹⁹F NMR (470 MHz, CDCl₃): δ=−80.91 (s, 3F), −113.78 (s, 2F), −121.80 (s, 2F), −122.04 (s, 4F), −122.83 (s, 2F), −123.66 (s, 2F), −126.23 (s, 2F);

¹³C NMR (126 MHz, CDCl₃): δ=178.87, 173.50, 69.28, 66.74, 56.10, 39.17, 32.11, 30.48, 30.41.

### Exemplary Preparation of the Salt 2j3k

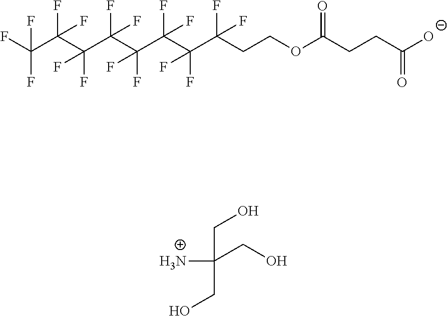 [SMILES: O=C([O-])CCC(=O)OCCC(F)(F)C(F)(F)C(F)(F)C(F)(F)C(F)(F)C(F)(F)C(F)(F)C(F)(F)F ; [NH3+]C(CO)(CO)CO]

In an exemplary method of preparation of the exemplary salt 2j3k, 1H,1H,2H,2H-perfluoro-1-decyl succinic acid monoester 2j 0.400 g (0.71 mmol) was dissolved in 2 mL of methanol and 0.086 g (0.71 mmol) of trizma-base (tri(hydroxymethyl)aminomethane) was added. After heating to complete dissolution, the mixture was cooled and concentrated to dryness to give the product 0.480 g (yield=99%).

### Spectral Analysis:

¹H NMR (500 MHz, CD₃OD): δ=4.37 (t, J=6.5 Hz, 2H), 3.64 (s, 6H), 2.66-2.53 (m, 4H), 2.47 (t, J=7.2 Hz, 2H);

¹⁹F NMR (470 MHz, CD₃OD): δ=−82.40 (s, 3F), −114.69 (s, 2F), −122.69 (s, 2F), −122.92 (s, 4F), −123.76 (s, 2F), −124.61 (s, 2F), −127.30 (s, 2F);

¹³C NMR (126 MHz, CD₃OD): δ=179.91, 174.60, 61.53, 57.33, 32.94, 31.48, 31.31.

### Exemplary Preparation of the Salt 2j3l

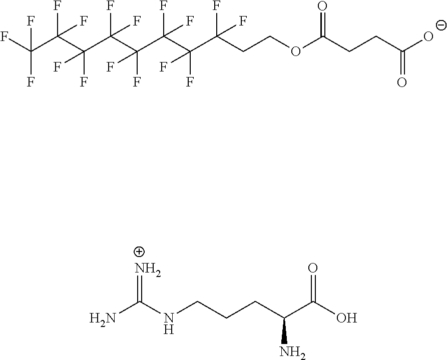 [SMILES: O=C([O-])CCC(=O)OCCC(F)(F)C(F)(F)C(F)(F)C(F)(F)C(F)(F)C(F)(F)C(F)(F)C(F)(F)F ; NC(=[NH2+])NCCC[C@H](N)C(=O)O]

In an exemplary method of preparation of the exemplary salt 2j3l, 1H,1H,2H,2H-perfluoro-1-decyl succinic acid monoester 2j 0.350 g (0.62 mmol) was dissolved in 2 mL of methanol and a solution of 0.108 g (0.62 mmol) L-arginine in water (0.5 mL) was added. After heating to complete dissolution, the mixture was cooled and concentrated to dryness to give the product 0.454 g (yield=99%).

### Spectral Analysis:

¹H NMR (500 MHz, D₂O): δ=4.38 (t, J=6.4 Hz, 2H), 3.75 (t, J=6.2 Hz, 1H), 3.24 (t, J=7.0 Hz, 2H), 2.59-2.43 (m, 6H), 1.96-1.87 (m, 2H), 1.78-1.63 (m, 2H);

¹⁹F NMR (470 MHz, D₂O): δ=−82.95-−83.18 (m, 3F), −114.69 (s, 2F), −122.74 (s, 2F), −123.01 (s, 2F), −123.12 (s, 2F), −124.04 (s, 2F), −124.40 (s, 2F), −127.76 (s, 2F);

¹³C NMR (126 MHz, D₂O): δ=179.65, 174.51, 174.41, 156.90, 56.46, 54.38, 40.62, 31.64, 30.13, 29.75, 27.77, 24.08.

### Exemplary Preparation of the Salt 2l3c

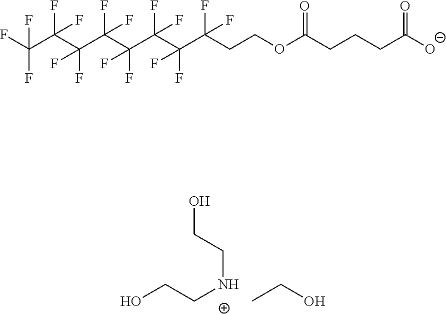

In an exemplary method of preparation of the exemplary salt 2l3c, 1H,1H,2H,2H-perfluoro-1-decyl glutaric acid monoester 2l 0.350 g (0.60 mmol) was dissolved in 2 mL of methanol and 0.090 g (0.60 mmol) of triethanolamine was added. After heating to complete dissolution, the mixture was cooled and concentrated to dryness to give the product 0.429 g (yield=98%).

### Spectral Analysis:

¹H NMR (500 MHz, D₂O/CD₃CN): δ=4.57 (t, J=6.4 Hz, 2H), 4.07 (t, J=5.4 Hz, 6H), 3.49 (t, J=5.4 Hz, 6H), 2.81-2.68 (m, 2H), 2.58 (t, J=7.6 Hz, 2H), 2.41 (t, J=7, 7 Hz, 2H), 2.09-1.99 (m, 2H);

¹⁹F NMR (470 MHz, D₂O/CD₃CN): δ=−82.46 (s, 3F), −114.33 (s, 2F), −122.45 (s, 2F), −122.79 (s, 4F), −123.68 (s, 2F), −124.17 (s, 2F), −127.34 (s, 2F);

¹³C NMR (126 MHz, D₂O/CD₃CN): δ=181.12, 175.19, 57.10, 56.78, 56.32, 36.88, 34.01, 30.59, 21.85.

### Exemplary Preparation of the Salt 2l3e

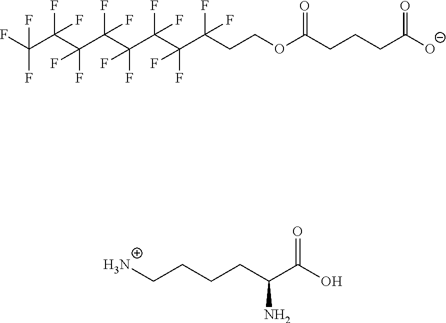 [SMILES: O=C([O-])CCCC(=O)OCCC(F)(F)C(F)(F)C(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2l3e, 1H,1H,2H,2H-perfluoro-1-decyl glutaric acid monoester 21 0.350 g (0.60 mmol) was dissolved in 2 mL of methanol and a solution of 0.088 g (0.60 mmol) L-lysine in water (0.5 mL) was added. After heating to complete dissolution, the mixture was cooled and concentrated to dryness to give the product 0.431 g (yield=99%).

### Spectral Analysis:

¹H NMR (500 MHz, D₂O/CD₃CN): δ=4.37 (t, J=6.3 Hz, 2H), 3.67 (t, J=6.2 Hz, 1H), 3.01 (t, J=7.5 Hz, 2H), 2.57-2.48 (m, 2H), 2.38 (t, J=7.5 Hz, 2H), 2.19 (t, J=7, 7 Hz, 2H), 1.90-1.80 (m, 4H), 1.75-1.66 (m, 2H), 1.55-1.39 (m, 2H);

¹⁹F NMR (470 MHz, D₂O/CD₃CN): δ=−83.02 (s, 3F), −114.65 (s, 2F), −122.74 (s, 2F), −123.00 (s, 2F), −123.10 (s, 2F), −124.02 (s, 2F), −124.43 (s, 2F), −127.74 (s, 2F);

¹³C NMR (126 MHz, D₂O/CD₃CN): δ=181.33, 175.51, 174.71, 56.39, 54.71, 39.15, 36.64, 33.25, 30.49, 29.76, 26.55, 21.60, 21.25.

Exemplary Preparation of the Salt 2l3k

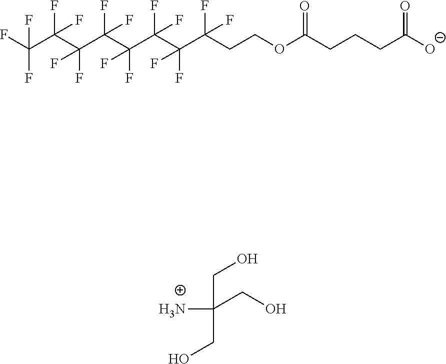 [SMILES: O=C([O-])CCCC(=O)OCCC(F)(F)C(F)(F)C(F)(F)C(F)(F)C(F)(F)C(F)(F)C(F)(F)C(F)(F)F ; [NH3+]C(CO)(CO)CO]

In an exemplary method of preparation of the exemplary salt 2l3k, 1H,1H,2H,2H-perfluoro-1-decyl glutaric acid monoester 21 0.350 g (0.60 mmol) was dissolved in 2 mL of methanol and 0.073 g (0.60 mmol) of trizma-base (tri(hydroxymethyl)aminomethane) was added. After heating to complete dissolution, the mixture was cooled and concentrated to dryness to give the product 0.416 g (yield=99%).

### Spectral Analysis:

¹H NMR (500 MHz, D₂O/CD₃CN): δ=4.61 (t, J=6.5 Hz, 2H), 3.92 (s, 6H), 2.78 (tt, J=18.8, 6.1 Hz, 2H), 2.61 (t, J=7.6 Hz, 2H), 2.44 (t, J=7.6 Hz, 2H), 2.10-2.02 (m, 2H);

¹⁹F NMR (470 MHz, D₂O/CD₃CN): δ=−82.44 (s, 3F), −114.29 (s, 2F), −122.38 (s, 2F), −122.70 (s, 4F), −123.61 (s, 2F), −124.10 (s, 2F), −127.25 (s, 2F);

¹³C NMR (126 MHz, D₂O/CD₃CN): δ=180.75, 174.64, 61.23, 60.15, 56.54, 36.39, 33.45, 30.03, 21.31.

### Exemplary Preparation of the Salt 2l3l

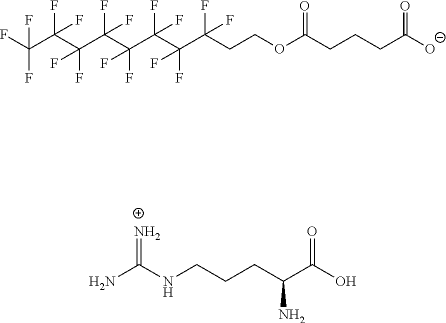 [SMILES: O=C([O-])CCCC(=O)OCCC(F)(F)C(F)(F)C(F)(F)C(F)(F)C(F)(F)C(F)(F)C(F)(F)C(F)(F)F ; NC(=[NH2+])NCCC[C@H](N)C(=O)O]

In an exemplary method of preparation of the exemplary salt 2131, 1H,1H,2H,2H-perfluoro-1-decyl glutaric acid monoester 21 0.350 g (0.60 mmol) was dissolved in 2 mL of methanol and a solution of 0.105 g (0.60 mmol) L-arginine in water (0.5 mL) was added. After heating to complete dissolution, the mixture was cooled and concentrated to dryness to give the product 0.448 g (yield=99%).

### Spectral Analysis:

¹H NMR (500 MHz, D₂O/CD₃CN): δ=4.49 (t, J=6.5 Hz, 2H), 3.83 (t, J=6.1 Hz, 1H), 3.35 (t, J=7.0 Hz, 2H), 2.71-2.58 (m, 2H), 2.50 (t, J=7.6 Hz, 2H), 2.31 (t, J=7.7 Hz, 2H), 2.07-1.91 (m, 4H), 1.89-1.74 (m, 2H);

¹⁹F NMR (470 MHz, D₂O/CD₃CN): δ=−82.85 (t, J=10.1 Hz, 3F), −114.08-−114.88 (m, 2F), −122.59 (s, 2F), −122.86 (s, 2F), −122.96 (s, 2F), −123.87 (s, 2F), −124.28 (s, 2F), −127.57 (s, 2F);

¹³C NMR (126 MHz, D₂O/CD₃CN): δ=181.92, 175.29, 175.26, 157.68, 57.08, 55.15, 41.41, 37.42, 34.03, 30.55, 28.65, 24.88, 22.03.

### Exemplary Preparation of the Salt 2k3c

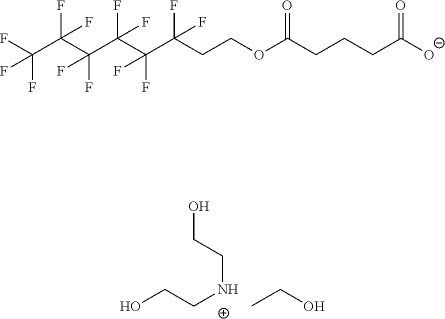

In an exemplary method of preparation of the exemplary salt 2k3c, 1H,1H,2H,2H-perfluoro-1-octyl glutaric acid monoester 2k 0.350 g (0.73 mmol) was dissolved in 2 mL of methanol and 0.109 g (0.73 mmol) of triethanolamine was added. After heating to complete dissolution, the mixture was cooled and concentrated to dryness to give the product 0.455 g (yield=99%).

### Spectral Analysis:

¹H NMR (500 MHz, D₂O/CD₃CN): δ=4.35 (t, J=6.4 Hz, 2H), 3.81 (t, J=5.6 Hz, 6H), 3.13 (t, J=5.1 Hz, 6H), 2.55-2.45 (m, 2H), 2.35 (t, J=7.6 Hz, 2H), 2.17 (t, J=7.7 Hz, 2H), 1.85-1.77 (m, 2H);

¹⁹F NMR (470 MHz, D₂O/CD₃CN): δ=−82.67 (s, 3F), −114.59 (s, 2F), −122.87 (s, 2F), −123.94 (s, 2F), −124.49 (s, 2F), −127.47 (s, 2F);

¹³C NMR (126 MHz, D₂O/CD₃CN): δ=181.05, 174.80, 56.71, 55.53, 36.31, 33.20, 32.10, 29.74, 21.08.

### Exemplary Preparation of the Salt 2k3e

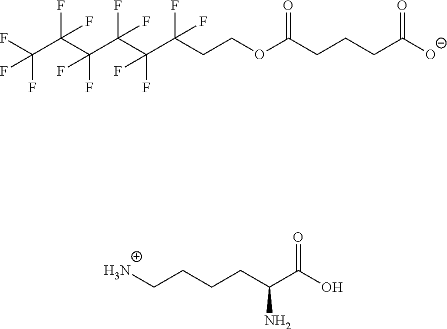 [SMILES: O=C([O-])CCCC(=O)OCCC(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2k3e, 1H,1H,2H,2H-perfluoro-1-octyl glutaric acid monoester 2k 0.350 g (0.73 mmol) was dissolved in 2 mL of methanol and a solution of 0.107 g (0.73 mmol) L-lysine in water (0.5 mL) was added. After heating to complete dissolution, the mixture was cooled and concentrated to dryness to give the product 0.453 g (yield=99%).

### Spectral Analysis:

¹H NMR (500 MHz, D₂O): δ=4.20 (t, J=6.4 Hz, 2H), 3.55 (t, J=6.1 Hz, 1H), 2.91-2.84 (m, 2H), 2.39-2.25 (m, 2H), 2.19 (dd, J=17.8, 10.3 Hz, 2H), 2.04 (dd, J=10.0, 5.3 Hz, 2H), 1.80-1.72 (m, 2H), 1.68-1.55 (m, 4H), 1.41-1.24 (m, 2H);

¹⁹F NMR (470 MHz, D₂O): δ=−83.02 (s, 3F), −114.98 (s, 2F), −123.09 (s, 2F), −124.19 (s, 2F), −124.83 (s, 2F), −127.78 (s, 2F);

¹³C NMR (126 MHz, D₂O): δ=184.05, 178.38, 177.40, 59.09, 57.20, 41.64, 39.02, 35.64, 33.00, 32.26, 29.08, 24.10, 23.65.

### Exemplary Preparation of the Salt 2k3k

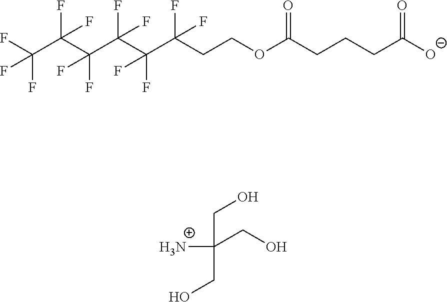 [SMILES: O=C([O-])CCCC(=O)OCCC(F)(F)C(F)(F)C(F)(F)C(F)(F)C(F)(F)C(F)(F)F ; [NH3+]C(CO)(CO)CO]

In an exemplary method of preparation of the exemplary salt 2k3k, 1H,1H,2H,2H-perfluoro-1-octyl glutaric acid monoester 2k 0.350 g (0.73 mmol) was dissolved in 2 mL of methanol and 0.088 g (0.73 mmol) of trizma-base (tri(hydroxymethyl)aminomethane) was added. After heating to complete dissolution, the mixture was cooled and concentrated to dryness to give the product 0.434 g (yield=99%).

### Spectral Analysis:

¹H NMR (500 MHz, D₂O/CD₃CN): δ=4.48 (t, J=6.4 Hz, 2H), 3.78 (s, 6H), 2.70-2.55 (m, 2H), 2.49 (t, J=7.6 Hz, 2H), 2.34-2.28 (m, 2H), 1.98-1.91 (m, 2H);

¹⁹F NMR (470 MHz, D₂O/CD₃CN): δ=−82.85 (t, J=10.1 Hz, 3F), −114.70 (s, 2F), −122.95 (s, 2F), −124.04 (s, 2F), −124.56 (s, 2F), −127.60 (s, 2F);

¹³C NMR (126 MHz, D₂O/CD₃CN): δ=181.30, 174.78, 60.77, 60.04, 56.43, 36.41, 33.19, 29.73, 21.11.

### Exemplary Preparation of the Salt 2k3

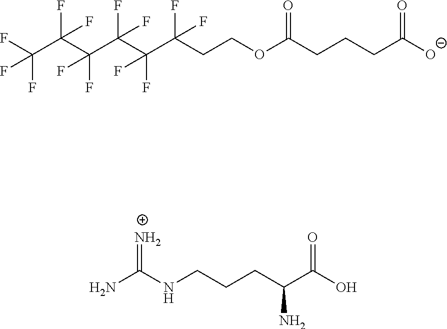 [SMILES: O=C([O-])CCCC(=O)OCCC(F)(F)C(F)(F)C(F)(F)C(F)(F)C(F)(F)C(F)(F)F ; NC(=[NH2+])NCCC[C@H](N)C(=O)O]

In an exemplary method of preparation of the exemplary salt 2k3l, 1H,1H,2H,2H-perfluoro-1-octyl glutaric acid monoester 2k 0.350 g (0.73 mmol) was dissolved in 2 mL of methanol and a solution of 0.127 g (0.73 mmol) L-arginine in water (0.5 mL) was added. After heating to complete dissolution, the mixture was cooled and concentrated to dryness to give the product 0.473 g (yield=99%).

### Spectral Analysis:

¹H NMR (500 MHz, D₂O/CD₃CN): δ=4.31 (t, J=6.4 Hz, 2H), 3.57 (t, J=6.2 Hz, 1H), 3.18 (t, J=6.9 Hz, 2H), 2.51-2.40 (m, 2H), 2.32 (t, J=7.5 Hz, 2H), 2.18-2.10 (m, 2H), 1.85-1.74 (m, 4H), 1.69-1.55 (m, 2H);

¹⁹F NMR (470 MHz, D₂O/CD₃CN): δ=−82.96 (t, J=10.1 Hz, 3F), −114.80 (s, 2F), −123.02 (s, 2F), −124.11 (s, 2F), −124.63 (s, 2F), −127.70 (s, 2F);

¹³C NMR (126 MHz, D₂O/CD₃CN): δ=181.47, 176.48, 174.71, 156.81, 56.37, 54.64, 40.68, 36.65, 33.18, 29.70, 28.68, 24.14, 21.19.

### Exemplary Preparation of the Salt 2p3c

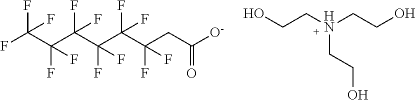

In an exemplary method of preparation of the exemplary salt 2p3c, 2H,2H-perfluorooctanoic acid (0.330 g, 0.87 mmol) was dissolved in 5 mL of methanol and 0.130 g (0.87 mmol) of triethanolamine was added. After heating to complete dissolution, the mixture was cooled and concentrated to dryness to give the product 0.454 g (99% yield).

### Spectral Analysis:

¹H NMR (500 MHz, acetone-d₆) δ=3.95-3.87 (m, 6H), 3.46-3.37 (m, 6H), 3.13 (t, J=19.5 Hz, 2H);

¹⁹F NMR (470 MHz, acetone-d₆) δ=−81.73 (td, J=10.0, 2.3 Hz, 3F), −112.71 (dt, J=19.3, 14.7 Hz, 2F), −118.56 (q, J=13.5 Hz, 2F), −123.25-−123.59 (m, 2F), −123.69 (s, 2F), −126.69-−127.05 (m, 2F);

¹³C NMR (126 MHz, acetone-d₆) δ=165.12, 57.08, 56.40, 37.58.

### Exemplary Preparation of the Salt 2p3e

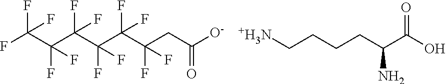 [SMILES: O=C([O-])CC(F)(F)C(F)(F)C(F)(F)C(F)(F)C(F)(F)C(F)(F)F ; N[C@@H](CCCC[NH3+])C(=O)O]

In an exemplary method of preparation of the exemplary salt 2p3e, 2H,2H-perfluorooctanoic acid (0.324 g, 0.86 mmol) was dissolved in 5 mL of methanol and a solution of 0.125 g (0.86 mmol) L-lysine in water (2 mL) was added. After heating to complete dissolution, the mixture was cooled and concentrated to dryness to give the product 0.446 g (99% yield).

### Spectral Analysis:

¹H NMR (500 MHz, acetone-d₆): δ=3.64 (t, J=6.0 Hz, 1H), 2.98-2.85 (m, 4H), 1.87-1.77 (m, 2H), 1.65 (dt, J=15.0, 7.6 Hz, 2H), 1.50-1.33 (m, 2H);

¹⁹F NMR (470 MHz, acetone-d₆): δ=−81.59 (t, J=9.9 Hz, 3F), −112.59-−113.12 (m, 2F), −122.36 (s, 2F), −123.43 (s, 2F), −123.67 (s, J=13.3 Hz, 2F), −126.75 (td, J=14.9, 6.7 Hz, 2F);

¹³C NMR (126 MHz, acetone-d₆): δ=173.82, 169.27, 54.45, 39.04, 38.34, 29.49, 26.38, 21.44.

Exemplary Preparation of the Salt 2r3c3c

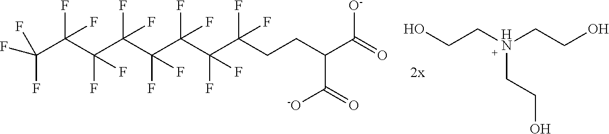

In an exemplary method of preparation of exemplary salt 2r3c3c, 2-(1H,1H,2H,2H-perfluorodecyl) malonic acid (0.300 g, 0.54 mmol) was dissolved in 5 mL of methanol and 0.162 g (1.08 mmol) of triethanolamine was added. After heating to complete dissolution, the mixture was cooled and concentrated to dryness to give the product 0.453 g (99% yield

### Spectral Analysis:

¹H NMR (500 MHz, D₂O/acetone-d₆) δ=4.03-3.93 (m, 12H), 3.51-3.34 (m, 13H), 2.31-2.18 (m, 2H), 2.16-2.02 (m, 2H);

¹⁹F NMR (470 MHz, D₂O/acetone-d₆): δ=−85.16 (t, J=10.3 Hz, 3F), −117.69 (p, J=18.3, 17.5 Hz, 2F), −124.98-−125.74 (m, 4F), −126.43 (s, 2F), −126.50 (d, J=19.9 Hz, 4F), −130.05 (dq, J=15.2, 7.2 Hz, 2F);

¹³C NMR (126 MHz, D₂O/acetone-d₆): δ=177.34, 56.03, 55.79, 49.77, 29.02, 21.06.

Exemplary Preparation of the Salt 2r3e3e

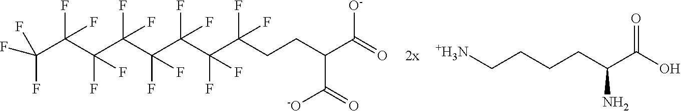

In an exemplary method of preparation of the exemplary salt 2r3e3e, 2-(1H,1H,2H,2H-perfluorodecyl) malonic acid (0.300 g, 0.54 mmol) was dissolved in 5 mL of methanol and a solution of 0.159 g (1.08 mmol) L-lysine in water (2 mL) was added. After heating to complete dissolution, the mixture was cooled and concentrated to dryness to give the product 0.450 g (99% yield).

### Spectral Analysis:

¹H NMR (500 MHz, D₂O/acetone-d₆) δ=3.79 (t, J=6.1 Hz, 2H), 3.13 (t, J=7.5 Hz, 1H), 3.09 (t, J=7.4 Hz, 4H), 2.26-2.14 (m, 2H), 2.04 (dt, J=11.4, 5.8 Hz, 2H), 2.00-1.88 (m, 4H), 1.84-1.74 (m, 4H), 1.63-1.44 (m, 4H);

¹⁹F NMR (470 MHz, D₂O/acetone-d₆): δ=−84.52-−86.56 (m, 3F), −116.68-−118.72 (m, 2F), −124.87-−125.34 (m, 2F), −125.48 (d, J=21.0 Hz, 4F), −125.67 (s, 2F), −126.20-−127.49 (m, 2F), −129.92-−130.78 (m, 2F);

¹³C NMR (126 MHz, D₂O/acetone-d₆): δ=178.09, 174.93, 57.69, 54.83, 39.41, 30.37, 29.14, 26.79, 21.85, 21.19.

### Exemplary Preparation of the Salt 2s3e

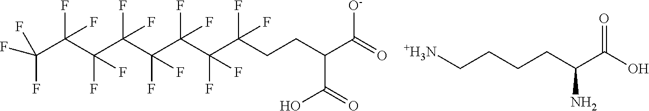 [SMILES: O=C([O-])C(CCC(F)(F)C(F)(F)C(F)(F)C(F)(F)C(F)(F)C(F)(F)C(F)(F)C(F)(F)F)C(=O)O ; N[C@@H](CCCC[NH3+])C(=O)O]

In an exemplary method of preparation of exemplary salt 2s3e, 2-(1H,1H,2H,2H-perfluorodecyl) malonic acid (0.300 g, 0.54 mmol) was dissolved in 5 mL of methanol and a solution of 0.79 g (0.54 mmol) L-lysine in water (1 mL) was added. After heating to complete dissolution, the mixture was cooled and concentrated to dryness to give the product 0.372 g (99% yield).

### Spectral Analysis:

¹H NMR (500 MHz, D₂O/acetone-d₆): δ=3.97-3.91 (m, 1H), 3.33-3.32 (m, 1H), 3.29 (t, J=11.5 Hz, 2H), 2.47-2.39 (m, 2H), 2.29-2.21 (m, 2H), 2.20-2.07 (m, 2H), 2.04-1.91 (m, 2H), 1.83-1.65 (m, 2H);

¹⁹F NMR (470 MHz, D₂O/acetone-d₆): δ=−81.38 (t, J=10.0 Hz, 3F), −114.59 (s, 2F), −122.17 (s, 2F), −122.39 (s, 4F), −123.22 (s, 2F), −123.52 (s, 2F), −126.60 (s, 2F);

¹³C NMR (126 MHz, D₂O/acetone-d₆): δ=178.09, 174.75, 57.64, 54.95, 49.19, 39.52, 30.56, 26.88, 21.92, 21.39.

Exemplary Preparation of the Salt 2r3k3k

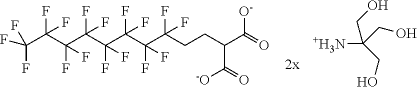

In an exemplary method of preparation of the exemplary salt 2r3k3k, 2-(1H,1H,2H,2H-perfluorodecyl) malonic acid (0.300 g, 0.54 mmol) was dissolved in 5 mL of methanol and 0.132 g (1.08 mmol) of trizma-base (tri(hydroxymethyl)aminomethane) was added. The mixture was heated until complete dissolution, then the mixture was cooled and concentrated to dryness to give the product 0.427 g (99% yield).

### Spectral Analysis:

¹H NMR (500 MHz, D₂O/acetone-d₆): δ=3.61 (s, 13H), 2.30-2.16 (m, 2H), 2.10-2.01 (m, 2H);

¹⁹F NMR (470 MHz, D₂O/acetone-d₆): δ=−80.56-−83.70 (m, 3F), −114.97 (dt, J=40.3, 22.6 Hz, 2F), −121.92-−122.61 (m, 2F), −122.81 (d, J=74.1 Hz, 4F), −123.79 (s, 2F), −127.16-−127.66 (m, 4F);

¹³C NMR (126 MHz, D₂O/acetone-d₆): δ=177.70, 60.60, 59.98, 56.67, 28.72, 20.77.

Exemplary Preparation of the Salt 2r3l3l

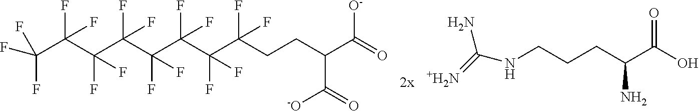

In an exemplary method of preparation of the exemplary salt 2r3l3l, 2-(1H,1H,2H,2H-perfluorodecyl) malonic acid (0.30 g, 0.54 mmol) 2r was dissolved in 5 mL of methanol and a solution of 0.19 g (1.08 mmol) L-arginine in water (0.2 mL) was added. After heating to complete dissolution, the mixture was cooled and concentrated to dryness to give the product 0.488 g (99% yield).

### Spectral Analysis:

¹H NMR (500 MHz, D₂O/acetone-d₆): δ=3.72 (t, J=6.1 Hz, 2H), 3.29 (t, J=6.9 Hz, 4H), 3.15 (t, J=7.5 Hz, 1H), 2.30-2.15 (m, 2H), 2.12-2.00 (m, 2H), 2.01-1.85 (m, 4H), 1.84-1.66 (m, 4H);

¹⁹F NMR (470 MHz, D₂O/acetone-d₆): δ=δ−81.96-−83.79 (m, 3F), −115.04 (s, 2F), −122.56 (s, 2F), −122.73-−123.05 (m, 4F), −123.67-−124.07 (m, 4F), −127.43 (s, 2F);

¹³C NMR (126 MHz, D₂O/acetone-d₆): δ=178.38, 176.30, 157.19, 58.05, 54.87, 49.20, 40.98, 28.84, 24.48, 21.42.

### Example III

### Manufacture of O/W Emulsion with the Newly Synthesized Surfactants

In an exemplary method of preparation, 180 mg of the surfactant was dissolved in 9 mL of ultrapure water (MilliQ), then 1 mL of Perfluorodecalin was added to the solution. The mixture was sonicated using an UP400St Hielscher ultrasonic homogenizer. Operation parameters of the device: A=90% (amplitude), continuous operation mode, sonotrode type—H14. The process of ultrasonic homogenization was carried out for about 2 minutes, while the reaction vessel was intensely cooled using an ice bath. 10 mL of the o/w emulsion with 10% v/v of the perfluorinated phase relative to the aqueous phase was obtained. As can be appreciated, this method is merely exemplary, and other methods or preparation approaches may be used.

### a) Determination of Particle Size Distribution of the Obtained O/W Emulsions

The emulsion particle size distribution was determined using the Accusizer 780 Optical Particle Sizer PSS NICOMP device. A series of dilutions of the analyzed emulsion were prepared: 10-fold, 100-fold and 1000-fold with ultrapure water (MilliQ), in triplicate. The particle size distribution of the 1000-fold diluted emulsion was examined, a minimum of 3 measurements were taken, preparing approximately 30 mL of the diluted emulsion for each one of them. The results were presented by the several values: the average diameter (given in μm), and the percentage number of particles in different ranges (for example, in the diameter range 0.5-2 μm, 2-5 μm, 5-10 μm, >10 μm). The values from these three measurements were averaged and standard deviation was calculated.

The particle size distribution of the emulsion was also measured by dynamic light scattering (DLS) using Malvern Zetasizer Nano ZS (Malvern Instruments. Ltd. Worcestershire. United Kingdom) device. A series of dilutions were prepared for each sample: 5-fold and 50-fold with ultrapure water (MilliQ). The measurements were carried out at the temperature of 25° C. and with a scattering angle of 173°. The results were presented using two parameters: average particle size and polydispersity index (PdI). The average particle size (given as the average diameter in nanometers) was the value which was calculated by the device, based on the particle intensity signal in accordance with the ISO standard that was provided by the manufacturer (ISO 13321: 1996E and 22412). The polydispersity index is a dimensionless value, expressing the width of the emulsion particle size distribution, i.e. the homogeneity of the analyzed sample, it was calculated in accordance with ISO 13321: 1996E and 22412. All measurements were carried out in triplicate. The values were given as the average value of three measurements and the standard deviation was calculated. Of course, this approach for determination of emulsion particle size distribution is exemplary, and other approaches may be used.

### b) Determination of the Zeta Potential of the Obtained O/W Emulsions Particles

The zeta potential measurements were taken using the Zetasizer Nano ZS Malvern device and the zeta potential measuring cell DTS1070. The obtained o/w emulsion was diluted 10-fold with ultrapure water (MilliQ). A minimum of 3 measurements were taken at the temperature of 25° C., taking about 2 mL of the diluted emulsion each time. Depending on the quality of the measurement was given by the device, diluted or undiluted emulsions were tested. The results for each sample were averaged, standard deviation was calculated, the zeta potential value was given in mV units. Of course, this approach for determination of zeta potential of the obtained O/W emulsion particles is exemplary, and other approaches may be used.

### c) Determination of Critical Micelle Concentration

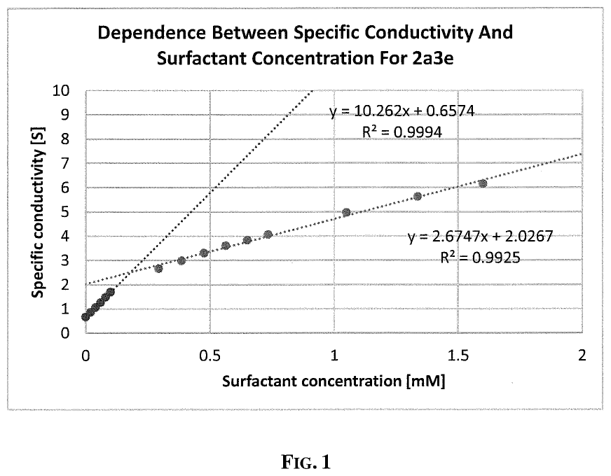

The Critical Micelle Concentration (CMC) was determined by the conductometric method, which consists of determining the difference in the change in the conductivity of the solution before and after the formation of micelles. Conductivity testing of the tested surfactants was carried out in the concentration range of 0.01-40 mM, at the temperature of 25° C. by adding a specific volume of concentrated surfactant solution to the water which conductivity was measured, thorough mixing of the solution using a magnetic stirrer (about 20 seconds at high speed) and measurement conductivity. The CMC point is visible as a refraction in the diagram of the dependence of conductivity on the surfactant concentration. See FIG. 1.

The exact CMC value was determined by specifying the intersection point of two trend lines along the points before and after the graph collapse and excluding the points on the bend, as shown in the diagram below. The measurements were taken to obtain at least 3 measurements after the CMC point lying on one straight line. The straight lines along different measurement points (before and after the CMC point) may slightly differ, creating a false impression of the graph collapsing, so it was assumed that to determine the real CMC value, the equations of two straight lines: y=a₁*x+b₁ and y=a₂*x+b₂, they had to meet the condition a₁/a₂>2. Of course, this approach for determination of the Critical Micelle Concentration is exemplary, and other approaches may be used.

TABLE 2
Summary of the results: average diameter, polydispersity
index, zeta potential for emulsions which were made
using the obtained surfactants and Critical Micelle
Concentration (CMC) for pure compounds.

| | | | Zeta | |
|---|---|---|---|---|
| | Average | Polydispersity | potential | CMC |
| Symbol | diameter [nm] | index [—] | [mV] | [mM] |
| 2a3a | 211.3 ± 12.0 | 0.276 ± 0.055 | −97.5 ± 0.7a | 0.98 |
| 2a3b | 640 ± 10 ^{c} | — | −82.3 ± 4.4a | 1.46 |
| 2a3c | 138.3 ± 2.1 | 0.039 ± 0.016 | −73.8 ± 2.3a | 1.74 |
| 2a3d | 640 ± 10 ^{c} | — | −23.0 ± 1.5a | 6.78 |
| 2a3e | 139.5 ± 1.4 | 0.082 ± 0.019 | −59.6 ± 0.9a | 0.21 |
| 2a3f | 175.1 ± 1.1 | 0.075 ± 0.019 | −20.8 ± 2.0 | 1.06 |
| 2a3g | 211.1 ± 1.3 | 0.145 ± 0.019 | −35.4 ± 1.2a | 0.97 |
| 2a3l | 148.9 ± 2.2 | 0.064 ± 0.024 | −55.7 ± 1.5a | 0.48 |
| 2aK | 770 ± 20 ^{c} | — | −67.7 ± 2.5a | 4 |
| 2b3c | 148.3 ± 1.7 | 0.046 ± 0.029 | −63.4 ± 1.4 | 2.97 |
| 2b3e | 143.5 ± 2.7 | 0.055 ± 0.017 | −51.5 ± 1.5 | 1.36 |
| 2b3f | 158.9 ± 2.1 | 0.087 ± 0.020 | −31.5 ± 1.8a | 1.98 |
| 2b3g | 277.2 ± 23.6 | 0.372 ± 0.043 | −45.8 ± 0.5a | 1.17 |
| 2b3l | 156.9 ± 1.9 | 0.057 ± 0.023 | −50.1 ± 0.8 | 0.67 |
| 2c3a | 687.5 ± 11.7 | 0.527 ± 0.031 | −88.1 ± 1.2a | 0.18 |
| 2c3c | 148.0 ± 1.3 | 0.064 ± 0.019 | −47.9 ± 0.3 | 0.27 |
| 2c3e | 160.0 ± 1.6 | 0.064 ± 0.020 | −60.9 ± 1.1a | 0.43 |
| 2c3f | 183.2 ± 0.9 | 0.072 ± 0.015 | −38.6 ± 1.8a | 0.98 |
| 2c3g | 278.7 ± 3.7 | 0.258 ± 0.023 | −41.7 ± 1.9a | 0.75 |
| 2c3k | 165.5 ± 1.4 | 0.059 ± 0.016 | −64.1 ± 1.7a | 1.6 |
| 2c3l | 148.1 ± 2.0 | 0.054 ± 0.018 | −55.3 ± 1.4a | 0.17 |
| 2cNa | 710 ± 20 ^{c} | — | −74.6 ± 2.2a | 1.62 |
| 2d3c | 144.4 ± 2.0 | 0.049 ± 0.021 | −64.2 ± 2.0a | 0.82 |
| 2d3e | 148.2 ± 2.0 | 0.059 ± 0.021 | −65.4 ± 3.7a | 0.95 |
| 2d3f | 157.8 ± 1.1 | 0.102 ± 0.021 | −36.6 ± 0.6a | 0.38 |
| 2d3g | 517.1 ± 18.8 | 0.591 ± 0.067 | −46.4 ± 1.4a | 0.59 |
| 2d3l | 156.9 ± 1.3 | 0.060 ± 0.012 | −66.9 ± 4.1 | 3.61 |
| 2i3c | 136.9 ± 1.8 | 0.064 ± 0.015 | −71.8 ± 2.4 | 4.66 |
| 2i3e | 145.1 ± 1.5 | 0.093 ± 0.016 | −49.7 ± 1.7a | 6.22 |
| 2i2i3h | 189.1 ± 1.1 | 0.103 ± 0.018 | −39.5 ± 1.0a | 1.3 |
| 2i2i3i | 201.1 ± 2.3 | 0.231 ± 0.014 | −39.9 ± 1.7a | 1.45 |
| 2i3k | 142.7 ± 1.5 | 0.065 ± 0.012 | −62.9 ± 0.9a | 4.08 |
| 2i3l | 143.0 ± 1.9 | 0.065 ± 0.017 | −54.6 ± 3.6a | 4.5 |
| 2iK | 880 ± 40 ^{c} | — | −95.1 ± 3.0a | 6.7 |
| 2j3a | 254.5 ± 5.2 | 0.333 ± 0.021 | −101 ± 7.3a | —^{b} |
| 2j3c | 146.6 ± 1.6 | 0.065 ± 0.016 | −66.3 ± 1.6a | 0.64 |
| 2j3e | 146.3 ± 1.3 | 0.059 ± 0.018 | −62.3 ± 1.6a | 1.07 |
| 2j2j3h | 170.8 ± 1.5 | 0.088 ± 0.018 | −30.2 ± 0.6a | 0.97 |
| 2j2j3i | 195.1 ± 2.6 | 0.147 ± 0.017 | −16.5 ± 0.8 | 1.83 |
| 2j3k | 142.2 ± 1.4 | 0.053 ± 0.014 | −66.4 ± 0.8a | 0.7 |
| 2j3l | 138.3 ± 2.0 | 0.065 ± 0.017 | −61.1 ± 0.4 | 0.63 |
| 2jK | 1090 ± 10 ^{c} | — | −85.6 ± 1.3a | 1.67 |
| 2jNa | 820 ± 20 ^{c} | — | −73.6 ± 2.6a | 1.64 |
| 2k3c | 148.8 ± 1.6 | 0.060 ± 0.012 | −66.0 ± 2.3a | 3.09 |
| 2k3e | 149.6 ± 1.3 | 0.055 ± 0.018 | −57.6 ± 1.4a | 2.1 |
| 2k3k | 150.8 ± 1.6 | 0.058 ± 0.021 | −64.4 ± 1.2a | 1.12 |
| 2k3l | 150.8 ± 1.9 | 0.047 ± 0.033 | −64.4 ± 1.0a | 0.97 |
| 2l3c | 162.8 ± 2.2 | 0.071 ± 0.045 | −64.3 ± 1.7a | 0.85 |
| 2l3e | 141.1 ± 1.4 | 0.061 ± 0.010 | −57.6 ± 2.4a | 0.92 |
| 2l3k | 156.0 ± 1.6 | 0.048 ± 0.020 | −69.8 ± 3.1a | 0.59 |
| 2l3l | 162.8 ± 1.8 | 0.052 ± 0.023 | −68.5 ± 0.9a | 0.37 |
| 2p3c | 150.8 ± 1.1 | 0.050 ± 0.030 | −65.9 ± 0.6 | 1.02 |
| 2p3e | 148.6 ± 0.5 | 0.050 ± 0.030 | −56.6 ± 1.0 | 0.7 |
| 2r3c3c | 147.9 ± 0.4 | 0.035 ± 0.003 | −61.7 ± 0.9 | 9 · 10⁻³ |
| 2r3e3e | 143.8 ± 1.1 | 0.060 ± 0.031 | −54.6 ± 0.7 | 1.4 · 10−3 |
| 2r3k3k | 151.0 ± 2.5 | 0.075 ± 0.021 | −86.9 ± 0.8 | 5 · 10⁻³ |
| 2r3l3l | 150.4 ± 2.0 | 0.135 ± 0.043 | −59.2 ± 1.8 | —^{b} |
| 2s3e | 143.9 ± 1.2 | 0.069 ± 0.016 | −60.9 ± 1.0 | 9 · 10⁻³ |

a—value of the zeta potential for 10-fold diluted emulsions;
^{b}water insoluble compound;
^{c} measurement was taken using the Accusizer 780 Optical ParticleSizer PSS NICOMP device

### Example IV

### In Vitro Cytotoxicity Study by XTT Assay

In vitro cytotoxicity of the compounds was tested according to a procedure, which has been developed based on the ISO 10993-5: 2009 (E) standard “Biological evaluation of medical devices—Part 5: Tests for in vitro cytotoxicity.”

Two cell lines were used in the experiments: L929—mouse fibroblasts and HMEC-1—human vascular endothelial cells. Fibroblasts were cultured in Dulbecco's Modified Eagle Medium (DMEM, 1 g/L glucose), endothelial cells in MCDB131 (1 g/L glucose); both culture media were supplemented with fetal bovine serum (10% FBS), antibiotics (1% Pen/Strep) and L-glutamine (2 mM DMEM, 10 mM MCDB131), MCDB131 additionally with hydrocortisone (1 μg/mL) and epidermal growth factor (EGF, 1 ng/mL). Cells were cultured in an incubator under standard conditions (37° C., 5% CO₂).

The tested compounds were weighed into two glass vessels and dissolved in both supplemented culture media. In case of water-insoluble compound, it was extracted at 37° C. for 22-24 hours, centrifuged, the supernatant was collected, sterilized by filtration (through a sterile 0.22 μm syringe filter) and then dilutions were prepared.

Both cell lines were seeded into 96-well plates at 5×10³ cells per well and incubated for 22-24 hours (37° C., 5% CO₂). Each plate contained (1) negative control (NC, cells in culture medium), (2) positive control (PC, cells treated with 2% Triton X-100 solution), (3) test sample (PR%, cells treated with previously prepared solutions/extracts) and (4) blank control (BL, each solution without cells). The day after seeding, 100 μl of solutions were applied onto the cells then plates were incubated for 20-24 hours (37° C., 5% CO2). On the last experimental day, cells' morphology was monitored using an inverted microscope, representative photos were taken and XTT assay was performed.

The XTT reagent solution was prepared in cell medium; immediately before use it was activated with PMS solution (Phenazine MethoSulfate=N-methyl dibenzopyrazine methylsulfate). Active XTT solution was added into the wells (NC, PC, PR %, BL), plates were incubated for 2 hours (37° C., 5% CO2) and spectrophotometric measurements were taken at two wavelengths λ1=450 nm and λ2=630 nm. Specific absorbance (A) of PR, NC, PC samples were calculated using A=A_{450Test}−A_{450Blank}−A_{630Test} equation. NC results were averaged (Ā_{NC}), and percentage of live cells in each well was determined using the formula:

$$PR \% = \frac{100 \cdot {\overline{A}}_{NC}}{{A}_{PR}}$$

Arithmetic average and standard deviation (SD) were calculated, the data were presented on graphs as cell viability (%) dependence on the concentration of the tested compound. Based on the results, the highest non-cytotoxic concentration of the compound was determined, where the cytotoxicity criteria was a decrease in cell viability below 70% compared to the negative control (100%).

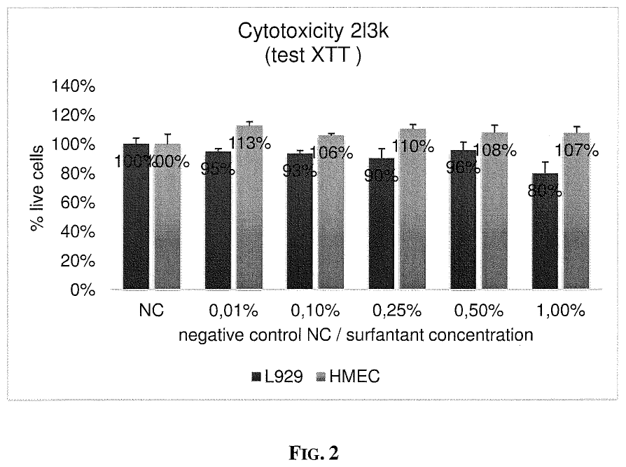

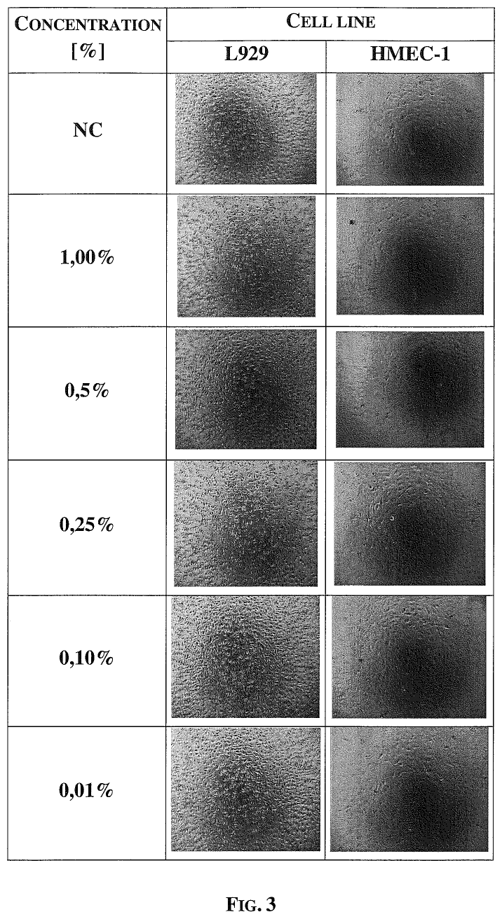

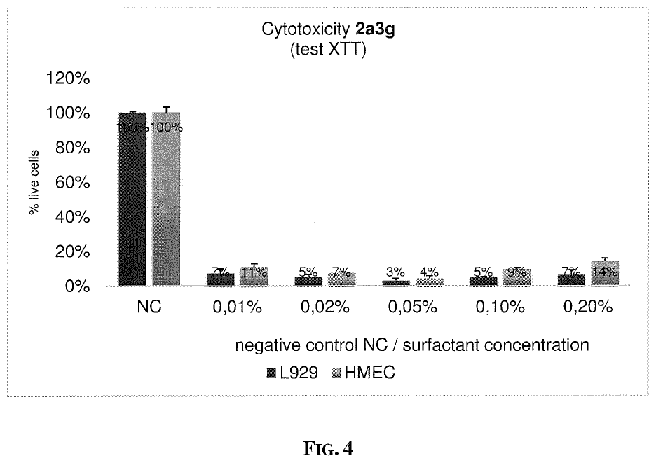

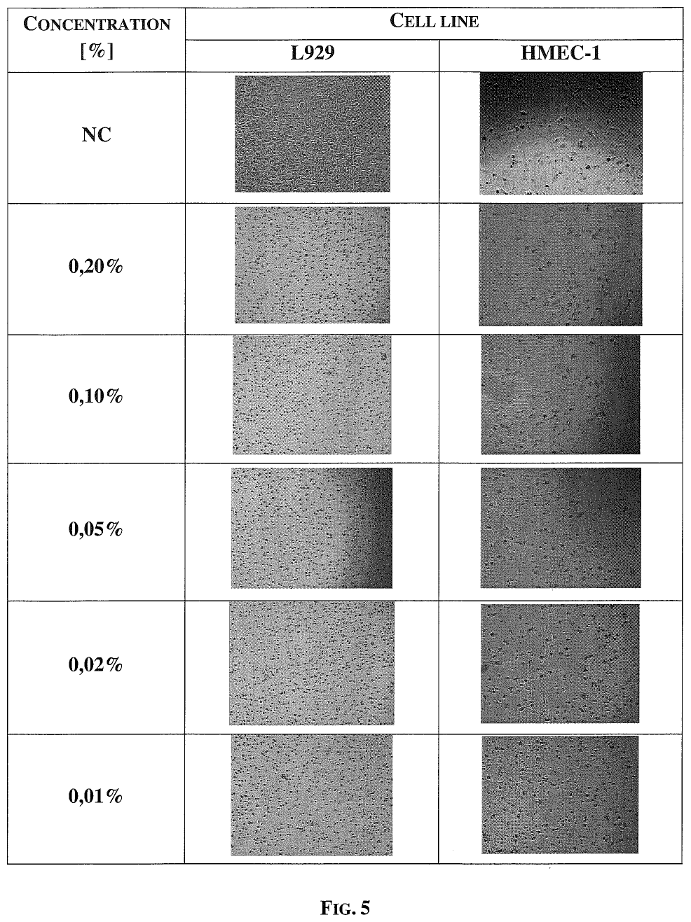

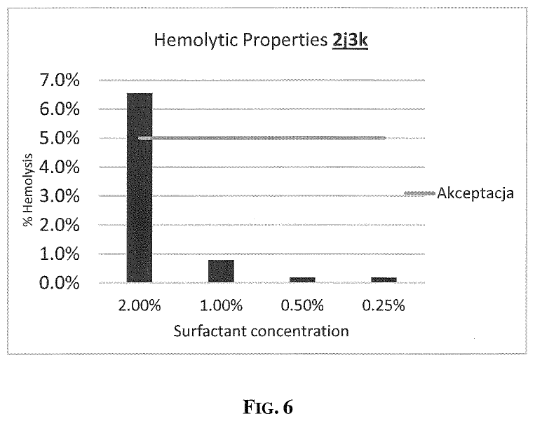

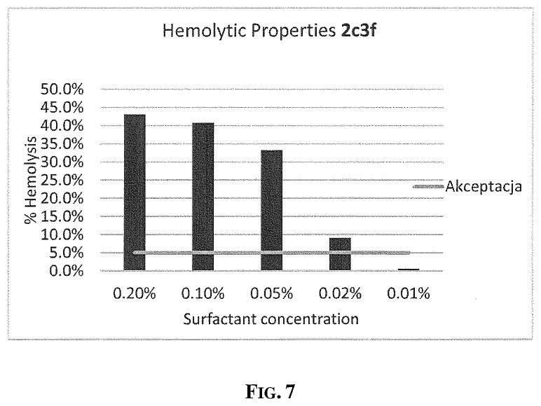

Exemplary sets of results for two compounds are presented later in the document.
Compound with low cytotoxic potential—2l3k. For graphics, see FIGS. 2 and 3
Compound with high cytotoxic potential—2a3g. For graphics, see FIGS. 4 and 5

Of course, this approach for testing the in vitro cytotoxicity of the compound is exemplary, and other approaches may be used.

### Example V

### Study of Hemolytic Properties of the New Surfactants

The study of hemolytic properties was conducted by adapting the method described in ASTM International Standard E2524-08: Test Method for Analysis of Hemolytic Properties of Nanoparticles. The method is listed as one of the tests in Practice F748 and ISO 10993-4, used to assess the biocompatibility of medical materials contacting with blood.

The assay is based on the quantification of hemoglobin released into the supernatant after blood exposing to a tested solution.

In the method, hemoglobin and its derivatives are oxidized to methemoglobin by ferricyanide at alkaline pH. The addition of a cyanide-containing Drabkin solution (also called a CMH reagent) converts methaemoglobin to cyanomethemoglobin (CMH). CMH as the most stable form of hemoglobin can be detected by spectrophotometric measurements at 540 nm wavelength. The addition of CMH reagent to the blood allows estimation of total hemoglobin (TBH), addition of CMH reagent to plasma allows estimation of the amount of hemoglobin released into the plasma (PFH).

Calibrators: Calibrators for the calibration curve plot were prepared from lyophilized human hemoglobin by a series of dilutions from 0.8 mg/mL to 0.025 mg/mL. Controls: Triton X-100 at 10 mg/mL was used as the positive control, the 40% polyethylene glycol solution was the negative control.

Of course this approach for studying the hemolytic properties of the exemplary new surfactants is exemplary, and other approaches may be used.

Examples of hemolytic properties test results:

2j3k compound; the result—no hemolytic properties for the compound at 1% and lower concentrations—see FIG. 4.

TABLE 6
Results of cytotoxicity and hemolysis studies of the compounds.

| | Range Of | | | Range Of | |
|---|---|---|---|---|---|
| | Concentration | Maximum Non- | | Concentration | Maximum Non- |
| | Tested In The | Cytotoxic | | Tested In The | Hemolytic |
| | Cytotoxicity | Concentration [%] | | Hemolysis | Concentration |
| Symbol | Assay [%] | L929 | HMEC-1 | Assay [%] | [%] |
| 2a3a | 2.00-0.01 | 0.5 | 1 | 0.20-0.01 | 0.05 |
| 2a3b | 2.00-0.25 | 0.5 | 0.5 | 2.00-0.25 | 0.25 |
| 2a3c | 2.00-0.01 | 0.2 | 0.2 | 2.00-0.25 | 0.2 |
| 2a3d | 2.00-0.01 | 0.2 | 0.1 | 0.20-0.01 | 0.02 |
| 2a3e | 2.00-0.01 | 0.2 | 0.2 | 2.00-0.01 | 0.25 |
| 2a3f | 2.00-0.01 | 0.2 | 0.2 | 2.00-0.25 | >2.00 |
| 2a3g | 2.00-0.01 | <0.01 | <0.01 | 2.00-0.25 | 0.5 |
| 2a3l | 1.00-0.01 | 0.5 | 0.5 | 2.00-0.01 | 1 |
| 2aK | 2.00-0.01 | 1 | 1 | 2.00-0.25 | >2.00 |
| 2b3c | 1.00-0.01 | 0.25* | 0.25 | 2.00-0.01 | 0.05 |
| 2b3e | 1.00-0.01 | 0.25 | 0.01 | 2.00-0.01 | 0.05 |
| 2b3f | 1.00-0.01 | 0.20** | 0.20** | 2.00-0.01 | 0.2 |
| 2b3g | 0.20-0.01 | <0.01*** | <0.01** | 2.00-0.01 | 0.1 |
| 2b3l | 1.00-0.01 | 0.10** | 0.25 | 0.20-0.01 | 0.05 |
| 2c3a | 2.00-0.25 | 0.5 | 0.5 | 0.20-0.01 | 0.02 |
| 2c3c | 2.00-0.25 | 0.25 | 0.5 | 2.00-0.25 | 0.25 |
| 2c3e | 2.00-0.25 | 0.5 | 1 | 0.20-0.01 | 0.02 |
| 2c3f | 2.00-0.25 | <0.25 | 1 | 0.20-0.01 | 0.01 |
| 2c3g | 2.00-0.01 | <0.01 | <0.01 | 0.20-0.01 | 0.1 |
| 2c3k | 2.00-0.01 | 0.25 | 1 | 0.20-0.01 | 0.05 |
| 2c3l | 1.00-0.01 | 0.25* | 0.5 | 0.20-0.01 | 0.02 |
| 2cNa | 2.00-0.01 | 1 | 0.2 | 2.00-0.25 | 0.5 |
| 2d3c | 1.00-0.01 | 0.01* | 0.01* | 2.00-0.01 | 0.01 |
| 2d3e | 1.00-0.01 | 0.01* | 0.01* | 2.00-0.01 | 0.01 |
| 2d3f | 1.00-0.01 | <0.01* | 0.5 | 2.00-0.01 | 0.01 |
| 2d3g | 1.00-0.01 | <0.01** | <0.01 | 2.00-0.01 | 0.01 |
| 2d3l | 1.00-0.01 | 0.01 | 0.25 | 0.20-0.01 | 0.01 |
| 2i3c | 2.00-0.01 | 0.10** | 0.10** | 0.20-0.01 | 0.02 |
| 2i3e | 2.00-0.01 | 0.20** | 0.10** | 0.20-0.01 | 0.05 |
| 2i2i3h | 2.00-0.01 | 0.1 | 0.1 | 0.20-0.01 | 0.05 |
| 2i2i3i | 2.00-0.01 | <0.01 | <0.01 | 0.20-0.01 | 0.05 |
| 2i3k | 2.00-0.01 | 0.1 | 0.1 | 0.20-0.01 | 0.1 |
| 2i3l | 1.00-0.01 | 0.01** | 0.01** | 2.00-0.01 | 0.05 |
| 2iK | 2.00-0.25 | <0.25** | 0.25 | 0.20-0.01 | 0.02 |
| 2j3a | 2.00-0.01 | 0.2 | 0.2 | 2.00-0.25 | >2.00 |
| 2j3c | 2.00-0.25 | 0.25 | 0.5 | 2.00-0.25 | >2.00 |
| 2j3e | 2.00-0.01 | 0.5 | 0.5 | 2.00-0.25 | 1 |
| 2j2j3h | 2.00-0.25 | 0.25** | 1 | 2.00-0.01 | >2.00 |
| 2j2j3i | 2.00-0.01 | <0.01 | <0.01 | 2.00-0.25 | >2.00 |
| 2j3k | 2.00-0.25 | 0.50* | 0.5 | 2.00-0.01 | 1 |
| 2j3l | 1.00-0.01 | 0.10** | 1 | 2.00-0.25 | >2.00 |
| 2jK | 2.00-0.25 | <0.25 | 0.5 | 2.00-0.25 | >2.00 |
| 2jNa | 2.00-0.25 | <0.25 | 0.5 | 2.00-0.25 | 1 |
| 2k3c | 1.00-0.01 | 0.10* | 0.10* | 2.00-0.01 | 0.05 |
| 2k3e | 1.00-0.01 | 0.10* | 0.01* | 2.00-0.01 | 0.05 |
| 2k3k | 2.00-0.01 | 0.20** | 0.10** | 2.00-0.01 | 0.05 |
| 2k3l | 2.00-0.25 | <0.25* | <0.25 | 0.20-0.01 | 0.05 |
| 2l3c | 1.00-0.01 | 0.5 | 0.5 | 2.00-0.25 | >2.00 |
| 2l3e | 2.00-0.01 | >2.00 | >2.00 | 2.00-0.25 | >2.00 |
| 2l3k | 1.00-0.01 | >1.00 | >1.00 | 2.00-0.25 | >2.00 |
| 2l3l | 1.00-0.01 | >1.00 | >1.00 | 2.00-0.25 | >2.00 |
| 2p3c | 2.00-0.01 | 0.1 | 0.1 | 2.00-0.01 | 0.25 |
| 2p3e | 2.00-0.01 | 0.1 | 0.25 | 2.00-0.01 | 0.25 |
| 2r3c3c | 2.00-0.25 | 0.5 | 0.5 | 2.00-0.01 | 0.2 |
| 2r3e3e | 2.00-0.25 | 0.25 | 0.25 | 2.00-0.01 | 0.2 |
| 2r3k3k | 2.00-0.25 | 0.5 | 0.5 | 2.00-0.01 | 0.1 |
| 2r3l3l | 2.00-0.25 | 0.25 | 0.5 | 2.00-0.01 | 0.05 |
| 2s3e | 2.00-0.25 | 0.25 | 0.25 | 2.00-0.01 | 0.1 |

*at the highest concentration to 0.50%, the compound crystallized/formed precipitate in the culture medium
**at the highest concentration to 0.25%, the compound crystallized/formed precipitate in the culture medium
***at the highest concentration to 0.10% the compound crystallized/formed precipitate in the culture medium
<non-toxic compound at lower concentration than tested
>non-toxic compound at the given concentration or higher than tested

### Example VII

### Red Blood Cells Substitute In Vivo Tests Including Administration of the Substances 2j3c, 2l3e, 2l3e to Rats Under General Anaesthesia

### Materials and Methods:

### Animals and Maintenance

The study protocol was approved by the Local Ethical Committee (No. WAW2/057/2018), Warsaw University of Life Sciences, Warsaw, Poland. Study was carried out by the Department of Large Animal Diseases with the Clinic of the Faculty of Veterinary Medicine of the Warsaw University of Life Sciences in Warsaw. The study involved male Sprague Dawley rats (n=19; weight, 400±20 g; age, 8 to 12 weeks) obtained from the Mossakowski Medical Research Centre, Polish Academy of Sciences (Warsaw, Poland) divided into 4 non-equal groups (table below). The animals were kept in accordance with national animal welfare guidelines. The animals showed no signs of disease either during the adaptation period or during the entire experiment

Assignment of the animals into research groups.

| | | Amount | | |
|---|---|---|---|---|
| | | Of The | Research | |
| Group | Test Substance | Animals | Method | Additional Analysis |
| I | Voluven + 2j3c | 5 | Exchanging 5% | Blood test |
| II | Voluven + 2j3e | 5 | of the blood | (gasometry, morphology, |
| III | Voluven + 2l3e | 5 | volume with | CRP, liver enzymes, |
| IV | Voluven (control) | 4 | tested substance | glucose, lactate, creatinine) |
| | | | | Histopathological |
| | | | | (HE staining) analysis of |
| | | | | the liver, kidneys, lungs, |
| | | | | heart (ongoing) |

### Experiment Protocol

12 h before the experiment, feedstuffs were removed from animals' cages without restrictions to the water. Animals were weighted for circulating blood volume calculation according to Lee and Blaufox (1985) and anesthetized by intramuscular injection of the mixture of Ceptor—0.1 mL (Scanvet), Butomidor—0.1 mL (Richter Pharma AG) and Bioketan—0.1 mL (Vetoquinol). Within 2 min after injection, animals were placed in the dorsal position, on Thermo-Controlled Surgery Platform (Braintree Scientific, USA) and the catheter for blood collection was implanted.

A silicone catheter (Scientific Commodities INC, USA) was inserted into the left common jugular vein towards the heart and fastened with a tie (non-absorbable twisted strand, 4-0) for best fluid delivery and blood sampling. The catheter was kept filled with Ringer's fluid between the blood removals. The catheter's patency was maintained without the use of anti-clotting agents. The skin was not stitched, a swab moistened with saline solution was applied to the wound. Then the rats were transferred to a GN 1/1 electric heating plate (temp. 37±1° C., Bartscher, Poland), where a cuff was applied to the rat's tail to measure bloodless tail blood pressure (MLT125R, ADI, Australia), connected to the system NIBP (Non-Invasive Blood Pressure, ADI, Australia) and eight-channel PowerLab (ADI, Australia) and PC. Throughout the experiment, pulse and blood pressure were recorded in the Chart program (ADI, Australia). During the entire experiment, systolic and mean arterial blood readings were taken up to a blood pressure systolic threshold reading of 40 mm Hg between blood collections.

A total of 4 experiment variants were performed, as in the table above, using the method of gradual bleeding with compensation described in the previous experiment (5% of circulating blood). Compensations were used in subsequent experiments: Voluven fluid (Hydroxyethyl starch (HES 130/0.4), dextran 130 kDa, Fresenius Kabi) and formulations 2j3c, 2j3e and 2l3e (NanoSanguis) administered in Voluven fluid (concentration 0.5% w/v). The experiment on each animal was conducted under anesthesia until cardiac cessation and heart rate ceased, hence the duration of the study and the number of samples taken may be one of the important factors used to infer about the action of the tested substances.

In venous blood samples, blood gas determinations (Siemens EPOC gasometer) were performed: pH, pCO₂ (CO₂ pressure, mm Hg), pO₂ (oxygen pressure, mm Hg), cHCO₃-(bicarbonate ion concentration, mmol/L), BE (ecf) (deficiency in blood buffering bases, mmol/L), hematocrit (Hct, %, less precise measurement than in a morphology analyzer), glucose (mg/dL), lactate (Lac, mmol/L), creatinine (Crea, mg/dL) followed by morphology (Mindray analyzer): hematocrit (hct, %), hemoglobin (g/L), red blood cell count (RBC, 10¹²/L), white blood cell count (WBC, ×10⁹/L) and platelet count (PLT, ×10⁹/L). Middleton et al. (2006) showed consistency in the results of gasometric measurements (pH, bicarbonate concentration, deficiency of buffering bases (Be), lactate concentration) from venous and arterial blood enabling the assessment of acid-base balance in the body. The results are given in the attached Excel spreadsheet. Blood for other biochemical analyses was centrifuged and frozen (−20° C.). Collected organ (liver, kidney, lung and heart) samples were fixed in buffered formalin, and after 48 hours. transferred to 70% ethyl alcohol and stored for histological examination. Histological examination was performed after routine staining of the hematoxylin and eosin sections.

Reference values of blood pressure and parameters in rats:

| | | | Sample 1: | |
|---|---|---|---|---|
| Parameter | Reference value | | X (min-max) | |
| Systolic blood pressure | 117 | (115-120) ^{a} | 143 | (105-171) |
| (mm Hg) | | | | |
| Mean blood pressure | No data | | 112 | (55-154) |
| (mm Hg) | | | | |
| pH | 7.33 | (7.25-7.38) ^{b} | 7.32 | (7.23-7.41) |
| pCO₂ (mm Hg) | 39.2 | (26-54) ^{b} | 71.9 | (54.2-83.6) |
| pO₂ (mm Hg) | 31.2 | (12-58) ^{b} | 33.2 | (25.1-37.8) |
| HCO₃⁻ ⁽mmol/L) | 20.8 | (12-58) ^{b} | 37.0 | (33.2-40.3) |
| Be(ecf) (mmol/L) | No data | | 10.9 | (7.2-13.9) |
| Hematokrit (%) | 41.6 | (1.3) ^{c} | 44.6 | (39.0-50.0) |
| RBC (×10¹²/L) | 7.01 | (0.38) ^{c} | 8.89 | (6.27-9.93) |
| Hemoglobin (g/L) | 142 | (5) ^{c} | 151 | (104-177) |
| WBC ((×10⁹/L) | 7.84 | (2.95) ^{c} | 4.2 | (2.4-5.4) |
| PLT (×10⁹/L) | 1141 | (91) ^{c} | 680 | (450-852) |
| Glucose (mg/dL) | 217 | (111-359) ^{b} | 402 | (223-491) |
| Lactate (mmol/L) | No data | | 0.40 | (0.30-0.91) |
| Creatynine (mg/dL) | No data | | 0.68 | (0.47-0.99) |
| Na⁺ (mmol/L) | 138.7 | (137.1-143.6) ^{d} | 142 | (136-147) |
| K⁺ (mmol/L) | 4.9 | (3.9-5.5) ^{d} | 5.2 | (4.1-6.7) |
| Ca⁺⁺ (mmol/L) | 1.0 | (0.8-1.1) ^{d} | 1.45 | (1.27-1.60) |
| Cl⁻ (mmol/L) | 98.9 | (97.9-103.8) ^{d} | 101 | (98-106) |

^{a} https://www.ncbi.nlm.nih.gov/pmc/articles/PMC4071721/https://www.ahajournals.org/doi/full/10.1161/JAHA.116.005204;
^{b} Uribe-Escamilla et al. 2011): Wistar rats 340 g, general anaesthesia (ketamine + xylazine), median (min-max);
^{c} Lillie et al. (1996): Sprague-Dawley rats, general anaesthesia (inhalation − halothane), abdominal aortic puncture, mean (SD);.
^{d} Baldissera et al. (2015), Wistar 200 g rats, general anaesthesia (inhalation − isoflurane), left ventricular puncture, median (min-max).

### Results

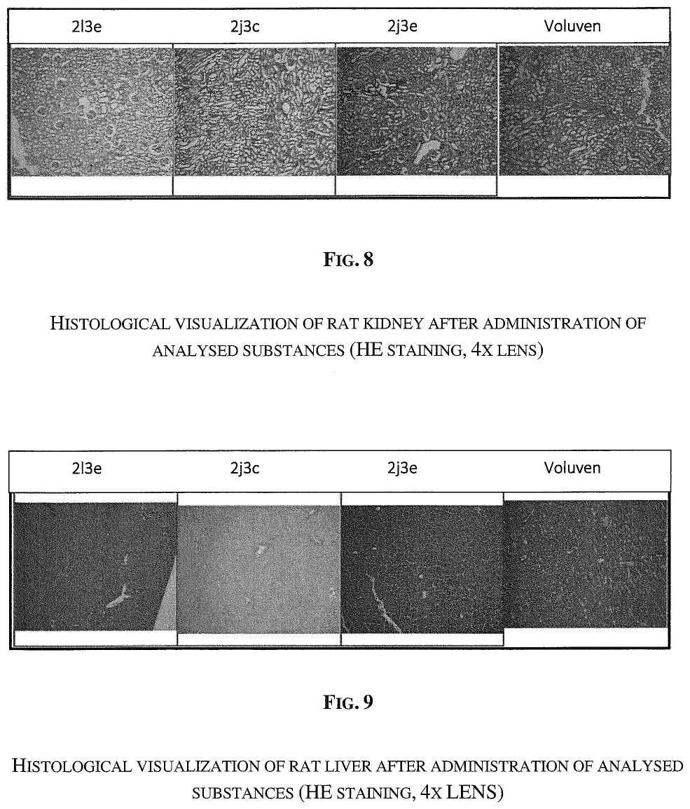

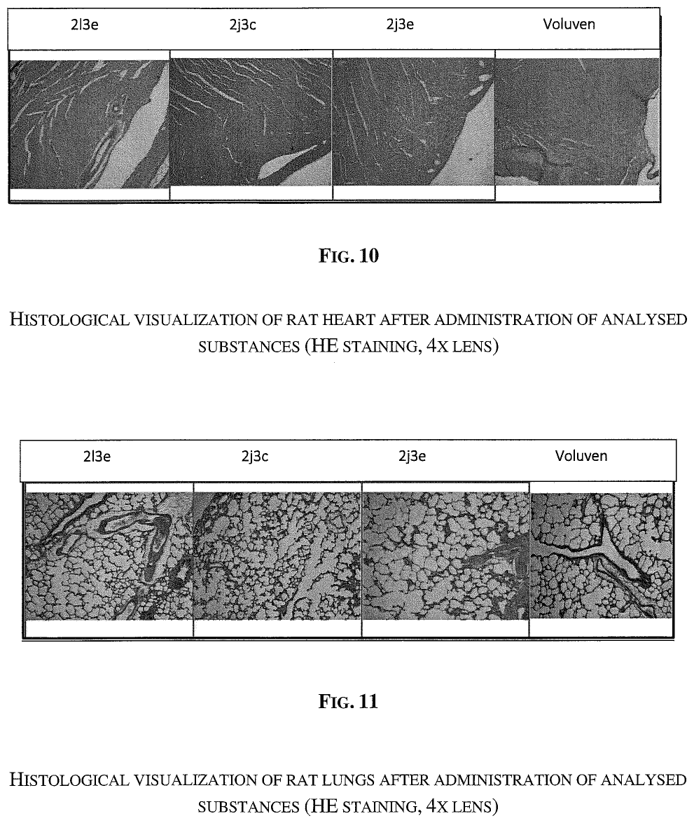

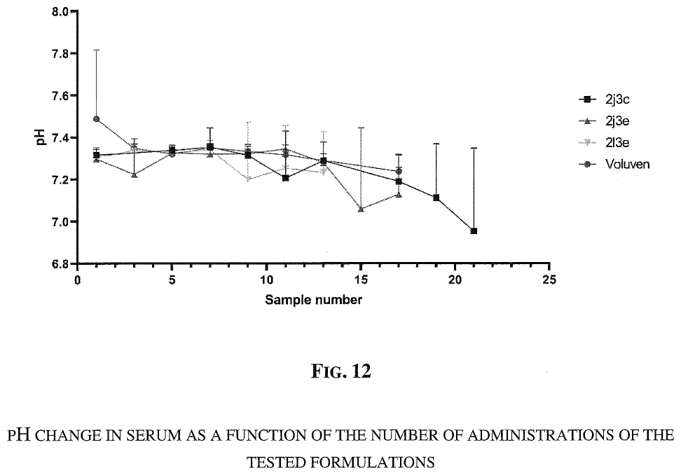

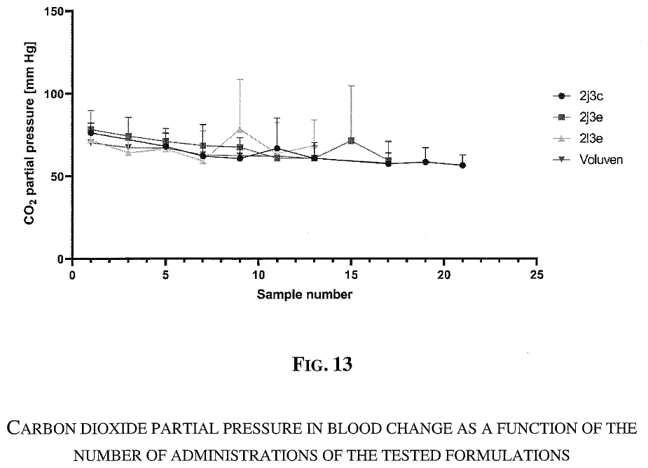

Macroscopic analysis during the section after the end of the experiment showed no pathological changes in the organs examined No congenital changes or those that could arise due to the administration of the preparations were observed. In the general picture, the pallor of organs was observed macroscopically proportional to the duration of the experiment. Representative microscopic images of organs are shown in FIGS. 8-11.

### Voluven

The amount of 5-minute intervals was 9, 18, 21 and 21 (i.e. 45, 90, 105 and 105 min). Blood pH, except for one animal, remained longer unchanged, i.e. up to the interval 8, 9, 15 and 15, similarly, systolic blood pressure was >40 mm Hg up to the interval 8, 9, 15 and 17. The base buffer balance (Be) was maintained for up to about 16 intervals (up to about 80 minutes). Hematocrit and other blood morphological parameters gradually decreased to the minimum value in the last interval in which the animals were still alive. Gradual increase in glucose in an intervals 13-15. An increase in lactate concentration corresponds to a decrease in Be.

Substance 2l3e+Voluven

Amount of 5-min intervals: 10, 13, 21, 28 and 31 (i.e. 60, 75, 105, 140 and 155 min). Systolic blood pressure was >40 mm Hg for 18-22 intervals. Significant differences were observed in the gasometric parameters (especially in pO₂, pCO₂ and concentration of buffering bases). The rats treated with the formulation in the second series showed gasometric and morphological changes in blood a few to several intervals later. A pH maintenance of 7.3 up to 20-21 intervals was observed. The collapse of buffering bases and an increase in lactate concentration corresponded to a decrease in pH. The decrease in morphological parameters corresponded to the blood loss.

Substance 2j3c+Voluven

Amount of 5-min intervals: 24, 23, 18, 18 and 11 (i.e. 120, 115, 90, 90 and 55 min). Systolic blood pressure was >40 mm Hg for 12, 20 intervals. The pH was maintained at 7.3 up to 13 and 17 intervals, and similarly, buffer balance (Be (ecf)) was disturbed. Hematocrit and the number of morphotic elements gradually decreased from physiological values to about 9% (Hct), 2×10¹²/L (RBC), 1×10⁹/L (WBC) at the end of the experiment. The increase in lactate concentration significantly above physiological values (limit value 8.5 mmol/L, Baldissera et al. 2015) occurred at intervals 15 and 19.

Substance 2j3e+Voluven

Amount of 5-min intervals: 15, 17, 21, 25 and 29 i.e. 75, 85, 105, 125 and 145 min). Systolic blood pressure maintained >40 mm Hg from 14 to 28 intervals, except that the dynamics of systolic pressure drop (rapid decrease at 10 initial intervals) was similar in all 5 rats, regardless of their survival time. Maintaining a pH of 7.3 was observed up to intervals 13 and 20, and buffer balance deficiency (Be (ecf)) occurred at 14-19 intervals. Hematocrit gradually decreased from physiological values to about 5%. A similar decrease was also observed in other blood morphotic parameters. Lactate concentration exceeded physiological breakpoints (8.5 mmol/L) at 13-19 intervals, although no change in lactate concentration was observed in 2 rats.

Summary (Charts from Averages for Each Measurement)

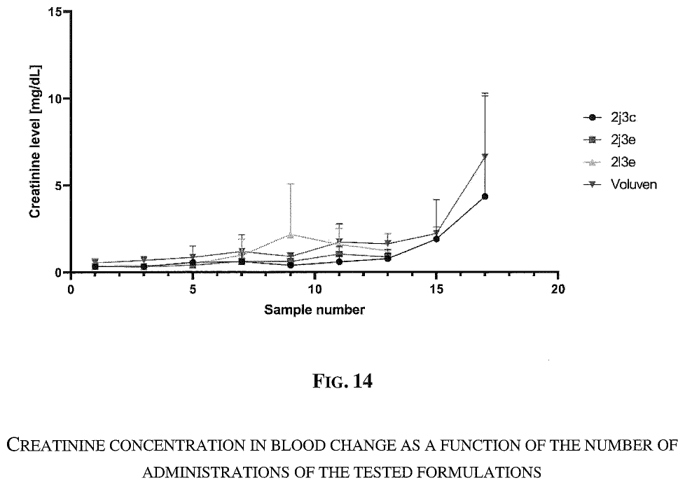

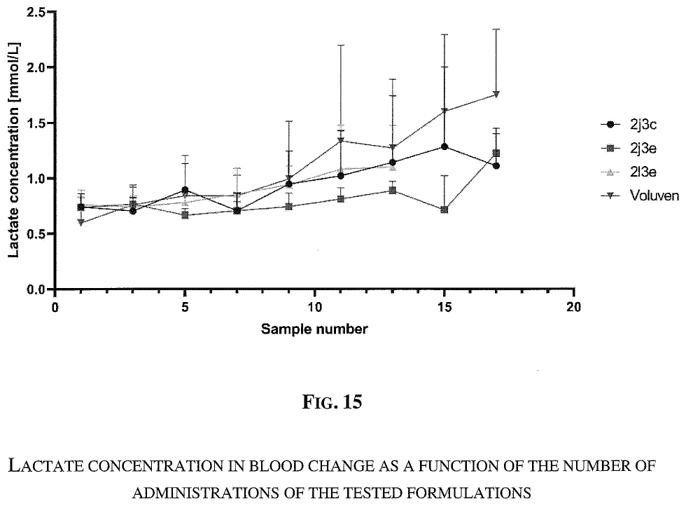

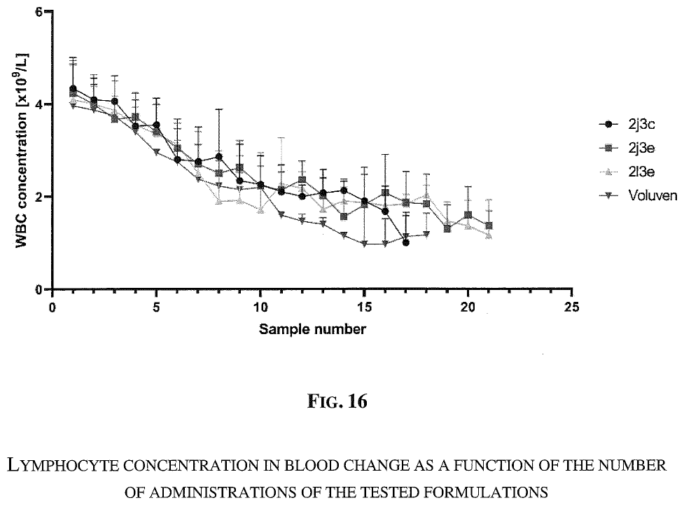

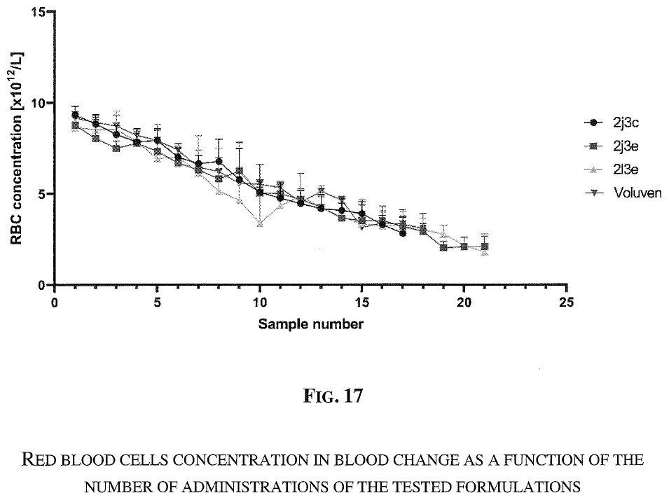

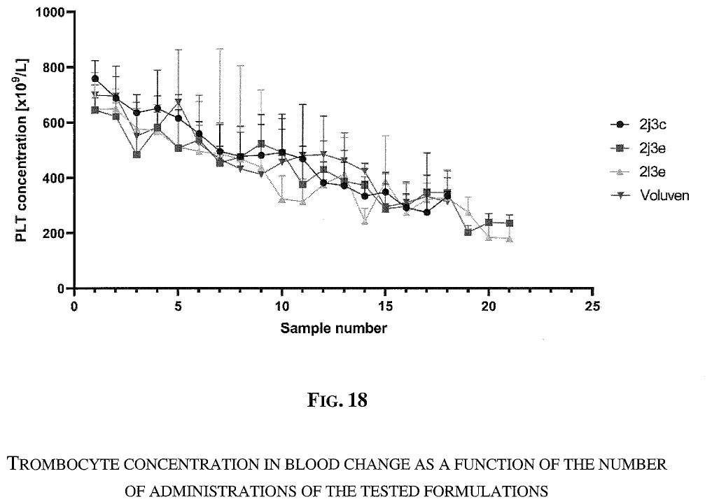

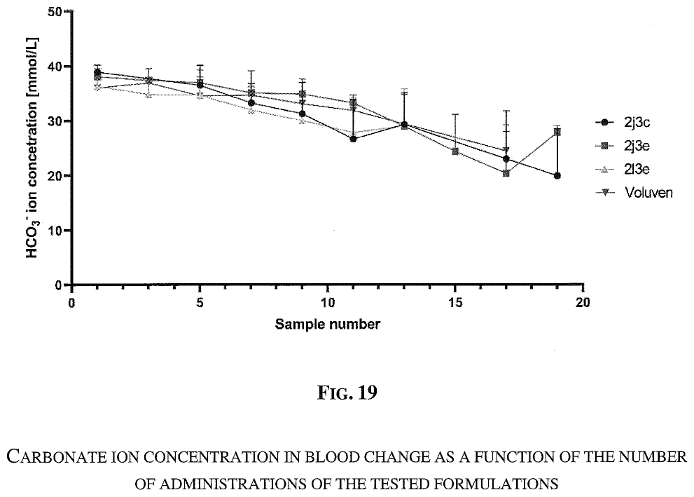

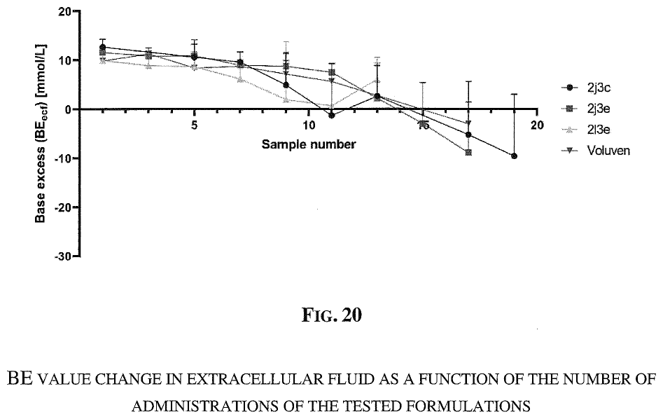

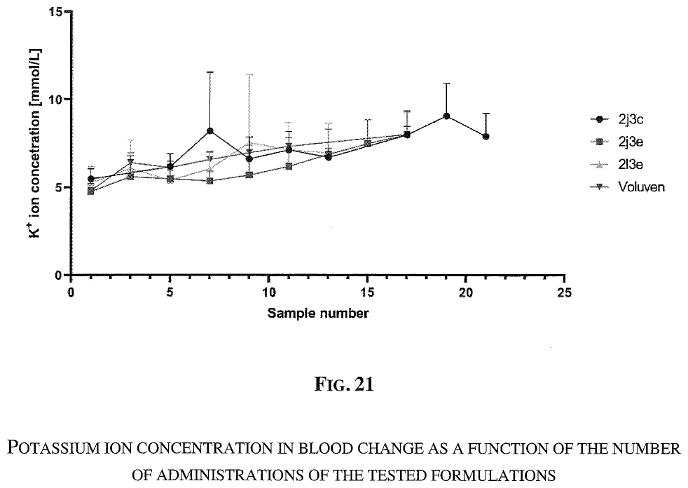

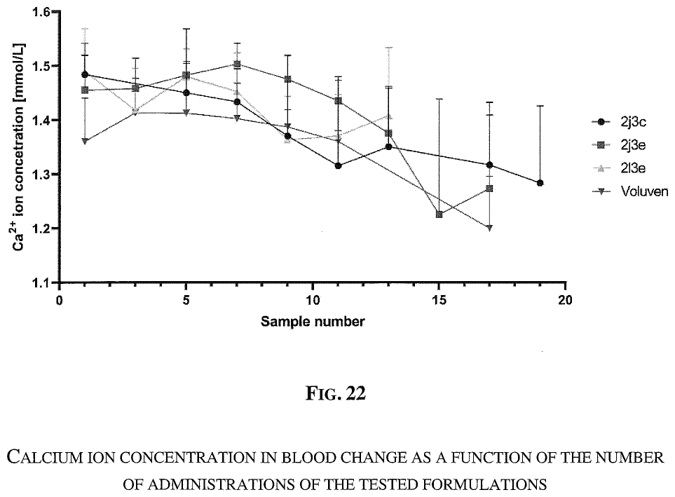

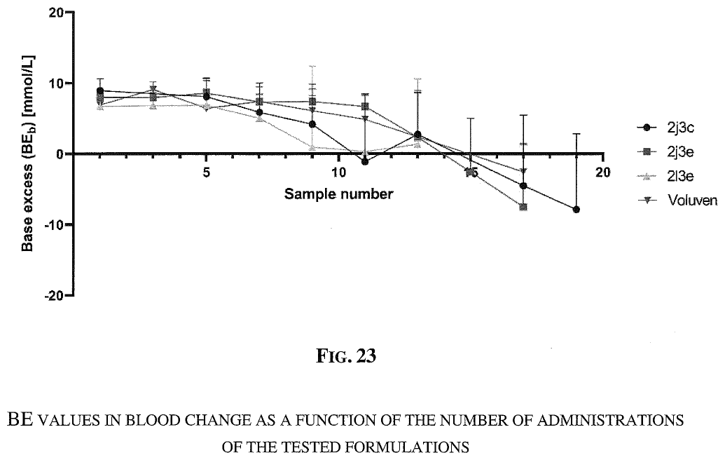

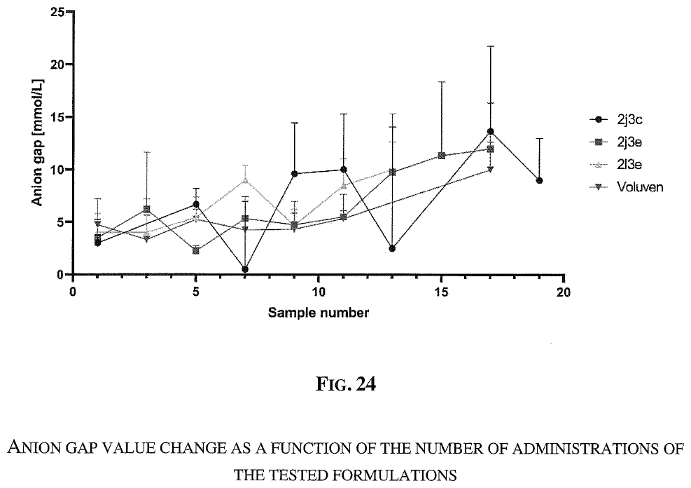

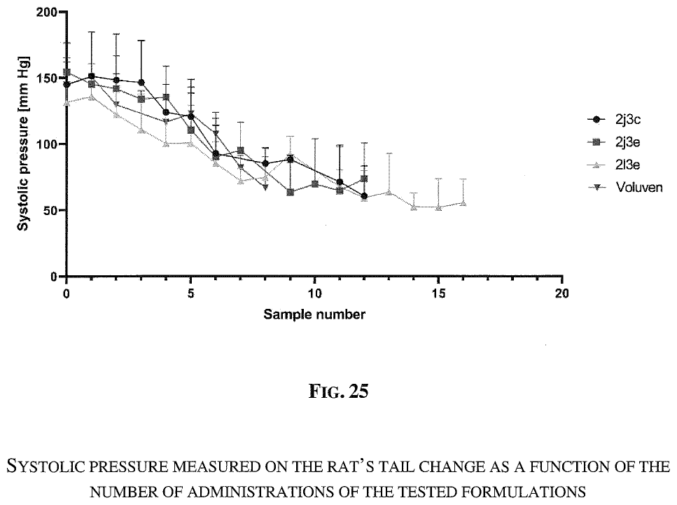

The results obtained are shown in FIGS. 12-25.

Bottom line: Survival of rats after administration of the tested formulations (2j3c, 2j3e, 2l3e) was 7-10 intervals higher compared to the control group receiving Voluven:

2l3e by 10 intervals,

2j3e by 8 intervals,

2j3c by 7 intervals.

At approximately 15 intervals, all test formulations caused a decrease in systolic pressure in the tail artery to a limit of 40 mm Hg, however for formulation 2j3e, maintaining pressure above the limit was longer comparing to other test groups. In comparison to the control group, the tested formulations caused slower:

pH decrease (for example, with use of 2l3e),

increase in pCO2 (for example, with use of 2l3e),

decrease in buffer base deficiency (for example, with use of 2l3e),

increase of lactate and creatinine (for example, with use of 2l3e).

No differences were found in blood counts, between the individual test formulations.

### Summary of Results of Blood Electrolytes

Measurement of electrolytes did not show concentration variation during blood compensation with the tested preparations. The concentration of sodium, potassium and chlorine gradually increased during the experiment at its end exceeding the upper limits.

It should be clear that the exemplary arrangements are not limited to the above presented arrangements and that diverse modifications, compounds, formulas, methods of synthesis, methods of use, and methods of application and developments thereof are possible.

Thus, the exemplary arrangements achieve improved compounds, formulas, methods of synthesis and use, and applications, eliminate difficulties encountered in the use of prior art compounds, formulas, methods of synthesis and use, and applications, and obtain the useful results described herein.

In the foregoing description, certain terms have been used for brevity, clarity, and understanding. However, no unnecessary limitations are to be implied therefrom because such terms are used for descriptive purposes only and are intended to be broadly construed. Moreover, the description and illustrations herein are by way of examples only, and the new and useful concepts are not limited to the exact features, compounds, formulas, methods, uses, and applications shown and described.

It should be understood that features and/or relationships, formulas, compounds, methods, uses, and applications associated with one arrangement can be combined with features and/or relationships, formulas, compounds, methods, uses, and applications from another arrangement. That is, various features and/or relationships, formulas, compounds, methods, uses, and applications from various arrangements can be combined in further arrangements. The inventive scope of the disclosure is not limited to only the arrangements shown or described herein.

Having described the features, discoveries, and principles of the exemplary arrangements, the manner and methods in which they are constructed, operated, synthesized, applied, and used and the advantages and useful results attained, the new and useful features, formulas, compounds, methods, combinations, applications, operations, uses, processes, and relationships are set forth in the appended claims.

1-29. (canceled)

30. A composition comprising:
an ammonium salt of partially fluorinated organic acids, represented by formula 1, wherein formula 1 comprises: 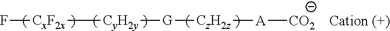
wherein
CₓF₂ₓ—comprises a straight chain, wherein X has a value within the range of 6 through 10,
C_{y}H_{2y}—comprises a straight chain, wherein Y has a value within the range of 1 through 2,
C_{z}H_{2z}—comprises a straight chain, wherein Z has a value within the range of 0 through 10,
G comprises one of
a bond,
a S atom, or
a carbonyloxy group (OCO),
A comprises one of
a bond,
a —(C(H)—COOH)— group, or
a —(C(H)—COO—)— group,
Cation (+) comprises one of
a 1,1,3,3-tetramethylguanidinium cation,
a lysinium cation,
an argininium cation,
a polylysinium cation,
a polycysteinium cation,
a polytyrosine cation, or 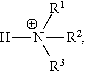
wherein each of R¹, R², R³ independently comprises one of
a hydrogen atom, an ethylenoxy group (—CH₂CH₂O—), a polyethylenoxy group (—CH₂CH₂O-)n, wherein n has a value within the range of 1 through 5, a C₁-C₁₀ alkyl group, a C₁-C₁₀ alkoxy group, a C₃-C₁₂ cycloalkyl group, an —N(R′)(R″) amine group, substituted with hydrogen atoms or at least one C₁-C₁₂ alkyl group, C₁-C₁₂ alkoxy, —N(R′)(R″) amine, —OR′ alkoxy group, wherein R, R′ and R″ comprise one of the same or different C₁-C₁₀ alkyl group, C₃-C₁₂ cycloalkyl group, C₁-C₁₀ alkoxy group.

31. The composition according to claim 30,
wherein an anion of the partially fluorinated carboxylic acid represented by formula 1 comprises one of 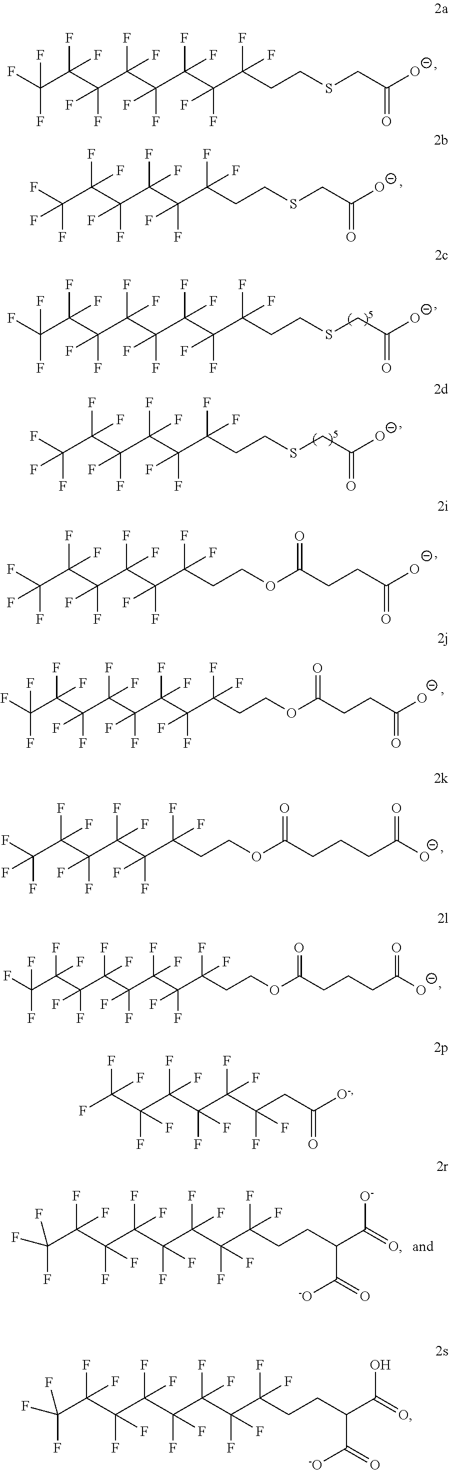
wherein the ammonium cation comprises one of

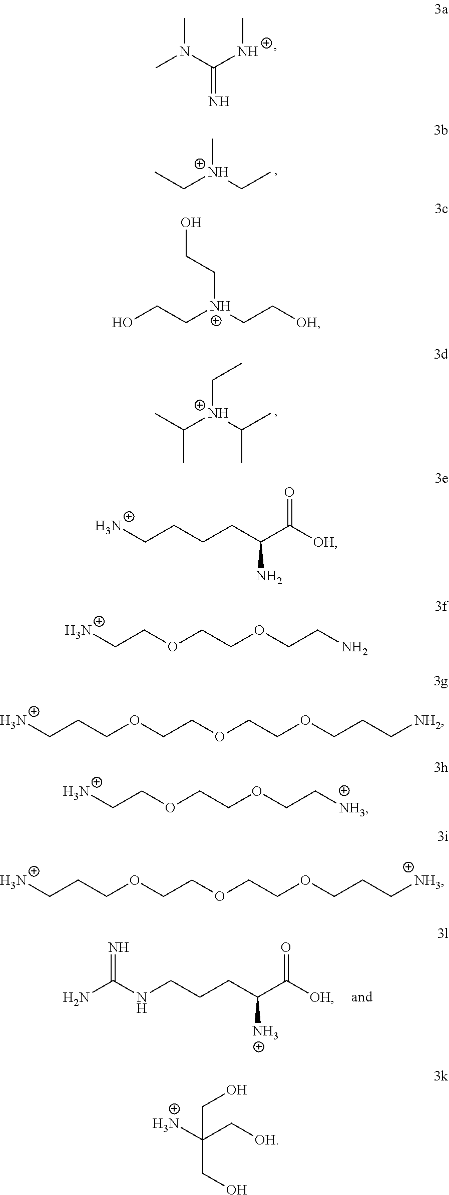

32. A method comprising:
a) providing a composition for an ammonium salt of partially fluorinated organic acids,
wherein the composition is represented by formula 1, wherein formula 1 comprises: 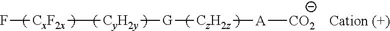
wherein
CₓF₂ₓ—comprises a straight chain, wherein X has a value within the range of 6 through 10,
C_{y}H_{2y}—comprises a straight chain, wherein Y has a value within the range of 1 through 2,
C_{z}H_{2z}—comprises a straight chain, wherein Z has a value within the range of 0 through 10,
G comprises one of
a bond,
a S atom, or
a carbonyloxy group (OCO),
A comprises one of
a bond,
a —(C(H)—COOH)— group, or
a —(C(H)—COO—)— group,
Cation (+) comprises one of
a 1,1,3,3-tetramethylguanidinium cation,
a lysinium cation,
an argininium cation,
a polylysinium cation,
a polycysteinium cation,
a polytyrosinium cation,
a potassium cation,
a sodium cation, or 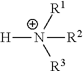
wherein each of R¹, R², R³ independently comprises one of
a hydrogen atom, an ethylenoxy group (—CH₂CH₂O—), a polyethylenoxy group (—CH₂CH₂O-)n, wherein n has a value within the range of 1 through 5, a C₁-C₁₀ alkyl group, a C₁-C₁₀ alkoxy group, a C₃-C₁₂ cycloalkyl group, an —N(R′)(R″) amine group, substituted with hydrogen atoms or at least one C₁-C₁₂ alkyl group, C₁-C₁₂ alkoxy, —N(R′)(R″) amine, —OR′ alkoxy group,
wherein R, R′ and R″ comprise one of the same or different C₁-C₁₀ alkyl group, C₃-C₁₂ cycloalkyl group, C₁-C₁₀ alkoxy group.

33. The method according to claim 32,
wherein in (a) an anion of the partially fluorinated carboxylic acid represented by formula 1 comprises one of 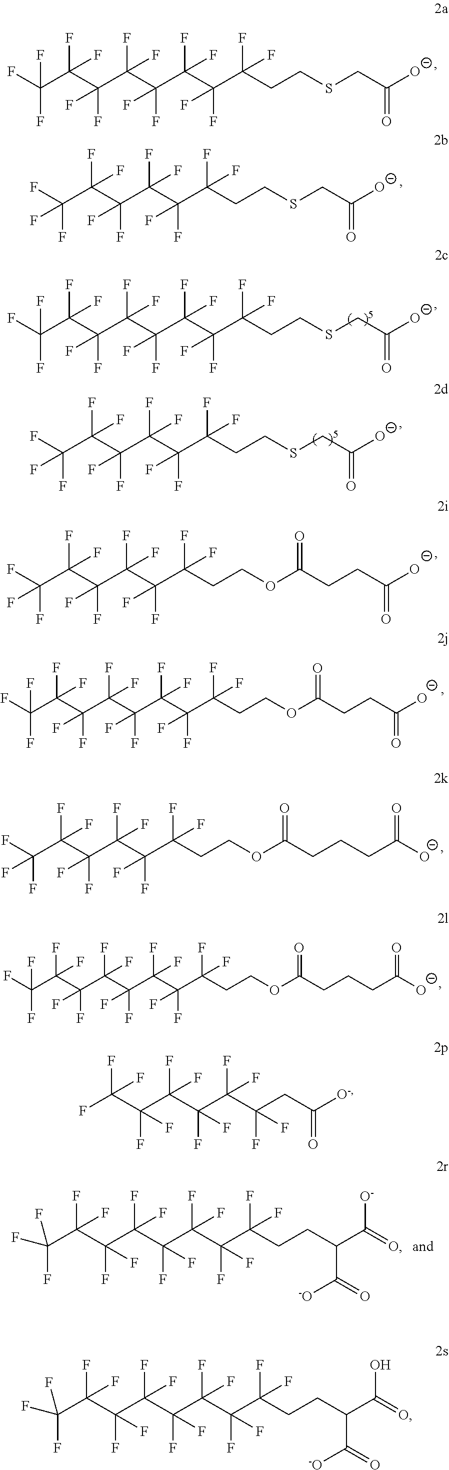
wherein the ammonium cation comprises one of

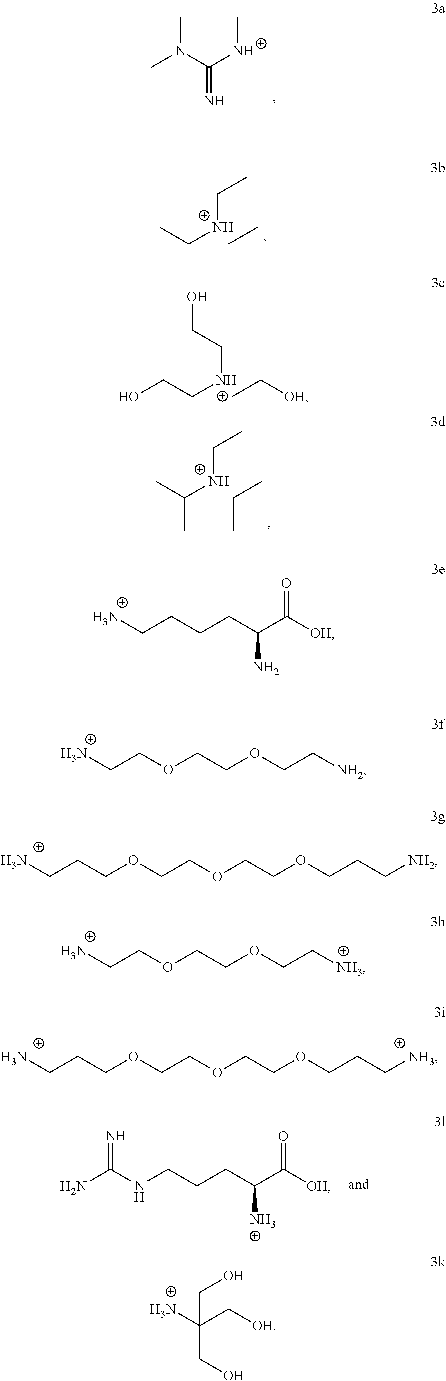

34. The method according to claim 32, and further comprising:
b) combining the composition in (a) with water and an oil,
c) subsequent and responsive at least in part to (b), producing an emulsion,
wherein gas is soluble in the emulsion of (c), and the emulsion has an emulsion particle size below 2 μm.

35. The method according to claim 32, and further comprising:
b) applying the composition provided in (a) to biological material,
c) subsequent to (b), storing the biological material to which the composition is applied in (b).

36. The method according to claim 32, and further comprising:
b) adding the composition in (a) to blood substitute preparations as a stabilising agent.

37. The method according to claim 32, and further comprising:
b) combining the composition in (a) with a liquid, wherein the liquid including the composition is operative to enable temporary support of respiration during artificial lung ventilation.

38. The method according to claim 32, and further comprising:
b) combining the composition in (a) with a further composition of a medical product.

39. The method according to claim 32, and further comprising:
b) combining the composition in (a) with a culture medium, wherein the culture medium including the composition is operative to deliver oxygen in bioreactors and aerobic organism cultures.

40. The method according to claim 32, and further comprising:
b) combining the composition in (a) with a culture medium, wherein the culture medium including the composition is operative to deliver carbon dioxide in bioreactors and anaerobic organism cultures.

41. A method comprising:
synthesizing a composition for an ammonium salt of partially fluorinated organic acids, represented by formula 1, wherein formula 1 is: 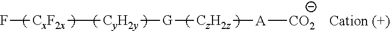
wherein
CₓF₂ₓ—comprises a straight chain, wherein X has a value within the range of 6 through 10,
C_{y}H_{2y}—comprises a straight chain, wherein Y has a value within the range of 1 through 2,
C_{z}H_{2z}—comprises a straight chain, wherein Z has a value within the range of 0 through 10,
G comprises one of
a bond,
a S atom, or
a carbonyloxy group (OCO),
A comprises one of
a bond,
a —(C(H)—COOH)— group, or
a —(C(H)—COO—)— group,
Cation (+) comprises one of
a 1,1,3,3-tetramethylguanidinium cation,
a lysinium cation,
an argininium cation,
a polylysinium cation,
a polycysteinium cation,
a polytyrosine cation, or 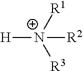
wherein each of R¹, R², R³ independently comprises one of
a hydrogen atom, an ethylenoxy group (—CH₂CH₂O), a polyethylenoxy group (—CH₂CH₂O-)n, wherein n has a value within the range of 1 through 5, a C₁-C₁₀ alkyl group, a C₁-C₁₀ alkoxy group, a C₃-C₁₂ cycloalkyl group, an —N(R′)(R″) amine group, substituted with hydrogen atoms or at least one C₁-C₁₂ alkyl group, C₁-C₁₂ alkoxy, —N(R′)(R″) amine, —OR′ alkoxy group, wherein R, R′ and R″ comprise one of the same or different C₁-C₁₀ alkyl group, C₃-C₁₂ cycloalkyl group, C₁-C₁₀ alkoxy group,
the method including:
a) reacting with at least one of an amine or an amino acid, a further partially fluorinated organic acid represented by formula 4, wherein formula 4 comprises: 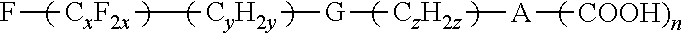
wherein
CₓF₂ₓ—comprises a straight chain, wherein X has a value within the range of 6 through 10,
C_{y}H_{2y}—comprises a straight chain, wherein Y has a value within the range of 1 through 2,
C_{z}H_{2z}—comprises a straight chain, wherein Z has a value within the range of 0 through 10,
G comprises one of
a bond,
a S atom, or
a carbonyloxy group (OCO),
A comprises one of
a bond,
a —(C(H)—COOH)— group, or
a —(C(H)—COO—)— group,
Cation (+) comprises one of
a 1,1,3,3-tetramethylguanidinium cation,
a lysinium cation,
an argininium cation,
a polylysinium cation,
a polycysteinium cation,
a polytyrosine cation, or 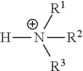
wherein each of R¹, R², R³ independently comprises one of
a hydrogen atom, an ethylenoxy group (—CH₂CH₂O—), a polyethylenoxy group (—CH₂CH₂O-)n, wherein n has a value within the range of 1 through 5, a C₁-C₁₀ alkyl group, a C₁-C₁₀ alkoxy group, a C₃-C₁₂ cycloalkyl group, an —N(R′)(R″) amine group, substituted with hydrogen atoms or at least one C₁-C₁₂ alkyl group, C₁-C₁₂ alkoxy, —N(R′)(R″) amine, —OR′ alkoxy group, wherein R, R′ and R″ comprise one of the same or different C₁-C₁₀ alkyl group, C₃-C₁₂ cycloalkyl group, C₁-C₁₀ alkoxy group.
wherein the reaction is operative to produce the organic acid represented by formula 1.

42. The method according to claim 41,
wherein the reaction in (a) is performed in the presence of at least one of
a solvent,
an alcohol comprising methanol, or
a mixture of alcohol comprising methanol and water.

43. The method according to 42,
wherein the amine or amino acid in (a) is added to the further partially fluorinated organic acid represented by formula 4, wherein the further organic acid has been dissolved in an alcohol comprising methanol in the form of at least one of
pure alcohol, or
an aqueous solution.

44. The method according to claim 43,
wherein the reaction mixture in (a) is heated to its respective boiling point.
